(19) United States
(12) Patent Application Publication
(10) Pub. No.: US 2023/0413823 A1
(43) Pub. Date: Dec. 28, 2023

(54) HYPERLOADED YEAST CELL WALL PARTICLE AND USES THEREOF

(71) Applicant: UNIVERSITY OF MASSACHUSETTS, Boston, MA (US)

(72) Inventors: Gary R. Ostroff, Worcester, MA (US); Ernesto R. Soto, Worcester, MA (US); Florentina Rus, Boston, MA (US)

(21) Appl. No.: 18/202,573

(22) Filed: May 26, 2023

Related U.S. Application Data
(60) Provisional application No. 63/346,012, filed on May 26, 2022.

(51) Int. Cl. A01N 43/80; A61K 47/46; A61K 31/085; A61K 31/05; A61K 31/045; A01N 47/10; A61K 31/015; A61K 31/00; A01N 37/34; A01P 1/00; A01P 3/00; A01N 43/56; A01N 43/22

(52) U.S. Cl. CPC A01N 43/80; A61K 47/46; A61K 31/085; A61K 31/05; A61K 31/045; A01N 47/10; A61K 31/015; A61K 31/658; A01N 37/34; A01P 1/00; A01P 3/00; A01N 43/56; A01N 43/22

(57) ABSTRACT

The present disclosure provides hyperloaded yeast particles. The disclosure further provides methods of making hyperloaded yeast particles and methods of using hyperloaded yeast particles.

### RELATED APPLICATIONS

The present invention claims the benefit of U.S. Provisional Application No. 63/346,012, filed May 26, 2022, the contents of which are incorporated herein by reference in its entirety for all purposes.

### FIELD OF THE INVENTION

The present disclosure relates to medicine, pharmacology, and agriculture. More specifically, the present disclosure relates to hyperloaded yeast cell wall particles comprising payloads.

### BACKGROUND

Drug delivery systems are designed to provide a biocompatible reservoir of an active agent for the controlled release of the active agent dependent either on time, or on local conditions, such as pH. There has been continuing interest in microscopic drug delivery systems such as microcapsules, microparticles and liposomes.

Yeast particles (YPs) are hollow, spherical particles about 2-4 μm in diameter that can be used for delivery of a drug payload. Due to their beta-glucan content, yeast particles can be targeted to phagocytic cells, such as macrophages and cells of lymphoid tissue. Use of yeast particles as drug delivery vehicles has been limited to payloads that are water soluble. See PCT Patent Application Publications WO2005/0281781, WO2007/050643, and WO2012/024229; United States Patent Application Publications US2009/0209624, US2013/0065941, US2014/0350066, and U.S. Pat. Nos. 9,662,299; and 5,032,401, 5,607,677, 7,740,861, 8,580,275, 8,389,485, 9,242,857, 9,662,299, and 9,682,135.

Encapsulation techniques for delivery of hydrophobic payloads include emulsification, extrusion, fluidized bed coating, spray drying, liposomes, molecular inclusion, coacervation, in situ polymerization, and nanostructured lipid matrices. Desirable qualities of a delivery systems are target specificity, absence of toxicity, high encapsulation efficiency, high loading capacity, homogenous distribution of payload in the payload carrier, low cost, mild processing conditions, improved storage stability, and controlled sustained release of payload.

YPs have been used to encapsulate payloads of low water solubility (0.1-2 mg/mL) by slow diffusion of the payloads through the pores of YPs forming an oil droplet inside the hydrophobic cavity of the YP. However, payload capacity was limited to <2:1 payload:YP weight ratio. The limited payload capacity of YPs has several disadvantages such as reduced therapeutic potency, increased shipping volume, and shorter duration of sustained payload release. Furthermore, methods known in the art for encapsulating payloads in YPs require a homogenization step, yield encapsulated payloads with limited stability, and do not allow controlled release of payload from YPs.

There is a need in the pharmaceutical and agricultural arts for the development of compositions and methods for delivering larger amounts of hydrophobic payloads to cells and organisms. YP delivery systems that encapsulate increased amount of payload, are stable, and allow controlled release of payload are needed.

### SUMMARY

In one aspect, a hyperloaded yeast particle (YP) is provided comprising a YP and a hydrophobic payload, wherein the hydrophobic payload is present within the YP, the weight by weight (w/w) ratio of the hydrophobic payload : the hyperloaded YP is about 2:1 to about 5:1, and the hydrophobic payload is releasable from the hyperloaded YP upon contact with an aqueous solution.

In certain exemplary embodiments, the YP is selected from the group consisting of a Biorigin YP, an SAF Mannan YP, a yeast cell wall particle (YCWP), a glucan particle (GP) and a mixture thereof.

In certain exemplary embodiments, the GP is selected from the group consisting of a yeast glucan particle (YGP), a yeast glucan-mannan particle (YGMP), a glucan lipid particle (GLP), a whole glucan particle (WGP) and a mixture thereof.

In certain exemplary embodiments, the hydrophobic payload comprises one or more hydrophobic compounds.

In certain exemplary embodiments, the hydrophobic payload is dissolved in an organic solvent. In certain exemplary embodiments, the organic solvent is selected from the group consisting of acetone, dichloromethane, ethyl acetate, ethanol, methanol, dimethyl sulfoxide (DMSO), eugenol, geraniol, a mixture of geraniol (G), eugenol (E), thymol (T) at a weight ratio composition of about 2:1:2 (GET424), N,N-dimethyl-decanamide (DMDA), octanoic acid, lauric acid, undecanoic acid, glycofurol, vitamin E, fatty acid, medium chain triglycerides (MCT), and a mixture thereof.

In certain exemplary embodiments, the organic solvent remains as a leave-in solvent in the hyperloaded YP.

In certain exemplary embodiments, the organic solvent is removed from the hyperloaded YP.

In certain exemplary embodiments, the hyperloaded YP further comprises a temperature stabilizing agent. In certain exemplary embodiments, the temperature stabilizing agent is glycerin.

In certain exemplary embodiments, the aqueous solution further comprises a surfactant. In certain exemplary embodiments, the surfactant is selected from the group consisting of sodium lauryl sulphate, polysorbate 20, polysorbate 80, polysorbate 40, polysorbate 60, polyglyceryl ester, polyglyceryl monooleate, decaglyceryl monocaprylate, propylene glycol dicaprilate, triglycerol monostearate, polyoxyethylenesorbitan, monooleate, TWEEN®, SPAN® 20, SPAN® 40, SPAN® 60, SPAN® 80, IGEPAL®, Triton X-100, Neobee, lecithin, Pluronic 31R1, Pluronic 17R4, Brij 30 and a mixture thereof.

In certain exemplary embodiments, the hydrophobic payload is selected from the group consisting of a terpene, a terpenoid, eugenol, geraniol, thymol, clomazone, triallate, limonene, lambda-cyhalotrin, penthiopyrad (PTP), spinosad, tetrahydrocannabinol (THC), cannabinol, cannabidiol, cannabigerol (CBG), aminoglycoside antibiotics, gentamycin, kanamycin, macrolides, erythromycin, rifamycins, novobiocin, fusidic acid, cationic peptides, cycloserine, rifampicin, doxorubicin, epirubicin, idarubicin, mitoxantrone, aspirin, acetaminophen, d-propoxyphene, fenoprofen, flurbiprofen, ibuprofen, ketoprofen, naproxen, naproxen-anhydride, oxaprozin, small organic active agent, a small inorganic active agent, a microbicide, a fungicide, an insecticide, a nematicide, a pesticide, an antibiotic, an analgesic, a non-steroidal anti-inflammatory drug (NSAID), a chemotherapeutic, a dietary supplement, and a mixture thereof.

In certain exemplary embodiments, the hyperloaded YP has a diameter that is greater than the diameter of a non-hyperloaded YP. In certain exemplary embodiments, the diameter of the hyperloaded YP is between about 6 μm and about 10 μm.

In another aspect, a pharmaceutical composition comprising a hyperloaded yeast particle (YP) and a pharmaceutically acceptable carrier or excipient is provided.

In certain exemplary embodiments, the YP is selected from the group consisting of a Biorigin YP, an SAF Mannan YP, a yeast cell wall particle (YCWP), a glucan particle (GP) and a mixture thereof.

In certain exemplary embodiments, the GP is selected from the group consisting of a yeast glucan particle (YGP), a yeast glucan-mannan particle (YGMP), a glucan lipid particle (GLP), a whole glucan particle (WGP) and a mixture thereof.

In certain exemplary embodiments, the hydrophobic payload comprises one or more hydrophobic compounds.

In certain exemplary embodiments, the hydrophobic payload is dissolved in an organic solvent. In certain exemplary embodiments, the organic solvent is selected from the group consisting of acetone, dichloromethane, ethyl acetate, ethanol, methanol, dimethyl sulfoxide (DMSO), eugenol, geraniol, a mixture of geraniol (G), eugenol (E), thymol (T) at a weight ratio composition of about 2:1:2 (GET424), N,N-dimethyl-decanamide (DMDA), octanoic acid, lauric acid, undecanoic acid, glycofurol, vitamin E, fatty acid, medium chain triglycerides (MCT), and a mixture thereof.

In certain exemplary embodiments, the organic solvent remains as a leave-in solvent in the hyperloaded YP.

In certain exemplary embodiments, the organic solvent is removed from the hyperloaded YP.

In certain exemplary embodiments, the hyperloaded YP further comprises a temperature stabilizing agent. In certain exemplary embodiments, the temperature stabilizing agent is glycerin.

In certain exemplary embodiments, the aqueous solution further comprises a surfactant. In certain exemplary embodiments, the surfactant is selected from the group consisting of sodium lauryl sulphate, polysorbate 20, polysorbate 80, polysorbate 40, polysorbate 60, polyglyceryl ester, polyglyceryl monooleate, decaglyceryl monocaprylate, propylene glycol dicaprilate, triglycerol monostearate, polyoxyethylenesorbitan, monooleate, TWEEN®, SPAN® 20, SPAN® 40, SPAN® 60, SPAN® 80, IGEPAL®, Triton X-100, Neobee, lecithin, Pluronic 31R1, Pluronic 17R4, Brij 30 and a mixture thereof.

In certain exemplary embodiments, the hydrophobic payload is selected from the group consisting of a terpene, a terpenoid, eugenol, geraniol, thymol, clomazone, triallate, limonene, lambda-cyhalotrin, penthiopyrad (PTP), prothioconazole (PRO), spinosad, tetrahydrocannabinol (THC), cannabinol (CBN), cannabidiol (CBD), cannabigerol (CBG), fish oil, aminoglycoside antibiotics, gentamycin, kanamycin, macrolides, erythromycin, rifamycins, novobiocin, fusidic acid, cationic peptides, cycloserine, rifampicin, doxorubicin, epirubicin, idarubicin, mitoxantrone, aspirin, acetaminophen, d-propoxyphene, fenoprofen, flurbiprofen, ibuprofen, ketoprofen, naproxen, naproxen-anhydride, oxaprozin, small organic active agent, a small inorganic active agent, a microbicide, a fungicide, an insecticide, a nematicide, a pesticide, an antibiotic, an analgesic, a non-steroidal anti-inflammatory drug (NSAID), a chemotherapeutic, a dietary supplement, and a mixture thereof.

In certain exemplary embodiments, the hyperloaded YP has a diameter that is greater than the diameter of a non-hyperloaded YP. In certain exemplary embodiments, the diameter of the hyperloaded YP is between about 6 μm and about 10 μm.

In another aspect, a method of preparing a hyperloaded yeast particle (YP) is provided comprising the steps of hydrating a YP with at least 0.5 μL aqueous solution per milligram of YP, and incubating the hydrated YP with a hydrophobic payload to encapsulate the hydrophobic payload within the YP.

In certain exemplary embodiments, the aqueous solution comprises a stabilizing agent. In certain exemplary embodiments, the stabilizing agent is glycerin.

In certain exemplary embodiments, the method further comprises a step of dissolving the hydrophobic payload in a solvent before incubating the hydrated YP.

In certain exemplary embodiments, the solvent is an organic solvent. In certain exemplary embodiments, the organic solvent is selected from the group consisting of acetone, dichloromethane, ethyl acetate, ethanol, methanol, dimethyl sulfoxide (DMSO), eugenol, geraniol, a mixture of geraniol (G), eugenol (E), thymol (T) at a weight ratio composition of about 2:1:2 (GET424), N,N-dimethyl-decanamide (DMDA), octanoic acid, lauric acid, undecanoic acid, glycofurol, vitamin E, fatty acid, medium chain triglycerides (MCT), and a mixture thereof.

In certain exemplary embodiments, the method further comprises a step of removing the solvent after the incubating step.

In certain exemplary embodiments, the weight by weight (w/w) ratio of the hydrophobic payload : the hyperloaded YP is between about 2:1 and about 5:1.

In certain exemplary embodiments, the hyperloaded YP has a diameter that is greater than the diameter of a non-hyperloaded YP. In certain exemplary embodiments, the diameter of the hyperloaded YP is between about 6 μm and about 10 μm.

In another aspect, a method of delivering a hydrophobic payload to a subject in need thereof is provided comprising administering to the subject a hyperloaded yeast particle (YP).

In certain exemplary embodiments, the YP is selected from the group consisting of a Biorigin YP, an SAF Mannan YP, a yeast cell wall particle (YCWP), a glucan particle (GP) and a mixture thereof.

In certain exemplary embodiments, the GP is selected from the group consisting of a yeast glucan particle (YGP), a yeast glucan-mannan particle (YGMP), a glucan lipid particle (GLP), a whole glucan particle (WGP) and a mixture thereof.

In certain exemplary embodiments, the hydrophobic payload comprises one or more hydrophobic compounds.

In certain exemplary embodiments, the hydrophobic payload is dissolved in an organic solvent. In certain exemplary embodiments, the organic solvent is selected from the group consisting of acetone, dichloromethane, ethyl acetate, ethanol, methanol, dimethyl sulfoxide (DMSO), eugenol, geraniol, a mixture of geraniol (G), eugenol (E), thymol (T) at a weight ratio composition of about 2:1:2 (GET424), N,N-dimethyl-decanamide (DMDA), octanoic acid, lauric acid, undecanoic acid, glycofurol, vitamin E, fatty acid, medium chain triglycerides (MCT), and a mixture thereof.

In certain exemplary embodiments, the organic solvent remains as a leave-in solvent in the hyperloaded YP.

In certain exemplary embodiments, the organic solvent is removed from the hyperloaded YP.

In certain exemplary embodiments, the hyperloaded YP further comprises a temperature stabilizing agent. In certain exemplary embodiments, the temperature stabilizing agent is glycerin.

In certain exemplary embodiments, the aqueous solution further comprises a surfactant. In certain exemplary embodiments, the surfactant is selected from the group consisting of sodium lauryl sulphate, polysorbate 20, polysorbate 80, polysorbate 40, polysorbate 60, polyglyceryl ester, polyglyceryl monooleate, decaglyceryl monocaprylate, propylene glycol dicaprilate, triglycerol monostearate, polyoxyethylenesorbitan, monooleate, TWEEN®, SPAN® 20, SPAN® 40, SPAN® 60, SPAN® 80, IGEPAL®, Triton X-100, Neobee, lecithin, Pluronic 31R1, Pluronic 17R4, Brij 30 and a mixture thereof.

In certain exemplary embodiments, the hydrophobic payload is selected from the group consisting of a terpene, a terpenoid, eugenol, geraniol, thymol, clomazone, triallate, limonene, lambda-cyhalotrin, penthiopyrad (PTP), spinosad, tetrahydrocannabinol (THC), cannabinol, cannabidiol, cannabigerol (CBG), aminoglycoside antibiotics, gentamycin, kanamycin, macrolides, erythromycin, rifamycins, novobiocin, fusidic acid, cationic peptides, cycloserine, rifampicin, doxorubicin, epirubicin, idarubicin, mitoxantrone, aspirin, acetaminophen, d-propoxyphene, fenoprofen, flurbiprofen, ibuprofen, ketoprofen, naproxen, naproxen-anhydride, oxaprozin, small organic active agent, a small inorganic active agent, a microbicide, a fungicide, an insecticide, a nematicide, a pesticide, an antibiotic, an analgesic, a non-steroidal anti-inflammatory drug (NSAID), a chemotherapeutic, a dietary supplement, and a mixture thereof.

In certain exemplary embodiments, the hyperloaded YP has a diameter that is greater than the diameter of a non-hyperloaded YP. In certain exemplary embodiments, the diameter of the hyperloaded YP is between about 6 μm and about 10 μm.

In another aspect, a composition for agricultural or environmental application is provided comprising a hyperloaded yeast particle (YP).

In certain exemplary embodiments, the YP is selected from the group consisting of a Biorigin YP, an SAF Mannan YP, a yeast cell wall particle (YCWP), a glucan particle (GP) and a mixture thereof.

In certain exemplary embodiments, the GP is selected from the group consisting of a yeast glucan particle (YGP), a yeast glucan-mannan particle (YGMP), a glucan lipid particle (GLP), a whole glucan particle (WGP) and a mixture thereof.

In certain exemplary embodiments, the hydrophobic payload comprises one or more hydrophobic compounds.

In certain exemplary embodiments, the hydrophobic payload is dissolved in an organic solvent. In certain exemplary embodiments, the organic solvent is selected from the group consisting of acetone, dichloromethane, ethyl acetate, ethanol, methanol, dimethyl sulfoxide (DMSO), eugenol, geraniol, a mixture of geraniol (G), eugenol (E), thymol (T) at a weight ratio composition of about 2:1:2 (GET424), N,N-dimethyl-decanamide (DMDA), octanoic acid, lauric acid, undecanoic acid, glycofurol, vitamin E, fatty acid, medium chain triglycerides (MCT), and a mixture thereof.

In certain exemplary embodiments, the organic solvent remains as a leave-in solvent in the hyperloaded YP.

In certain exemplary embodiments, the organic solvent is removed from the hyperloaded YP.

In certain exemplary embodiments, the hyperloaded YP further comprises a temperature stabilizing agent. In certain exemplary embodiments, the temperature stabilizing agent is glycerin.

In certain exemplary embodiments, the aqueous solution further comprises a surfactant. In certain exemplary embodiments, the surfactant is selected from the group consisting of sodium lauryl sulphate, polysorbate 20, polysorbate 80, polysorbate 40, polysorbate 60, polyglyceryl ester, polyglyceryl monooleate, decaglyceryl monocaprylate, propylene glycol dicaprilate, triglycerol monostearate, polyoxyethylenesorbitan, monooleate, TWEEN®, SPAN® 20, SPAN® 40, SPAN® 60, SPAN® 80, IGEPAL®, Triton X-100, Neobee, lecithin, Pluronic 31R1, Pluronic 17R4, Brij 30 and a mixture thereof.

In certain exemplary embodiments, the hydrophobic payload is selected from the group consisting of a terpene, a terpenoid, eugenol, geraniol, thymol, clomazone, triallate, limonene, lambda-cyhalotrin, penthiopyrad (PTP), spinosad, tetrahydrocannabinol (THC), cannabinol (CBN), cannabidiol (CBD), cannabigerol (CBG), aminoglycoside antibiotics, gentamycin, kanamycin, macrolides, erythromycin, rifamycins, novobiocin, fusidic acid, cationic peptides, cycloserine, rifampicin, doxorubicin, epirubicin, idarubicin, mitoxantrone, aspirin, acetaminophen, d-propoxyphene, fenoprofen, flurbiprofen, ibuprofen, ketoprofen, naproxen, naproxen-anhydride, oxaprozin, small organic active agent, a small inorganic active agent, a microbicide, a fungicide, an insecticide, a nematicide, a pesticide, an antibiotic, an analgesic, a non-steroidal anti-inflammatory drug (NSAID), a chemotherapeutic, a dietary supplement, and a mixture thereof.

In certain exemplary embodiments, the hyperloaded YP has a diameter that is greater than the diameter of a non-hyperloaded YP. In certain exemplary embodiments, the diameter of the hyperloaded YP is between about 6 μm and about 10 μm.

In another aspect, a kit is provided comprising a hyperloaded yeast particle (YP), and optional instructions for use.

In certain exemplary embodiments, the YP is selected from the group consisting of a Biorigin YP, an SAF Mannan YP, a yeast cell wall particle (YCWP), a glucan particle (GP) and a mixture thereof.

In certain exemplary embodiments, the GP is selected from the group consisting of a yeast glucan particle (YGP), a yeast glucan-mannan particle (YGMP), a glucan lipid particle (GLP), a whole glucan particle (WGP) and a mixture thereof.

In certain exemplary embodiments, the hydrophobic payload comprises one or more hydrophobic compounds.

In certain exemplary embodiments, the hydrophobic payload is dissolved in an organic solvent. In certain exemplary embodiments, the organic solvent is selected from the group consisting of acetone, dichloromethane, ethyl acetate, ethanol, methanol, dimethyl sulfoxide (DMSO), eugenol, geraniol, a mixture of geraniol (G), eugenol (E), thymol (T) at a weight ratio composition of about 2:1:2 (GET424), N,N-dimethyl-decanamide (DMDA), octanoic acid, lauric acid, undecanoic acid, glycofurol, vitamin E, fatty acid, medium chain triglycerides (MCT), and a mixture thereof.

In certain exemplary embodiments, the organic solvent remains as a leave-in solvent in the hyperloaded YP.

In certain exemplary embodiments, the organic solvent is removed from the hyperloaded YP.

In certain exemplary embodiments, the hyperloaded YP further comprises a temperature stabilizing agent. In certain exemplary embodiments, the temperature stabilizing agent is glycerin.

In certain exemplary embodiments, the aqueous solution further comprises a surfactant. In certain exemplary embodiments, the surfactant is selected from the group consisting of sodium lauryl sulphate, polysorbate 20, polysorbate 80, polysorbate 40, polysorbate 60, polyglyceryl ester, polyglyceryl monooleate, decaglyceryl monocaprylate, propylene glycol dicaprilate, triglycerol monostearate, polyoxyethylenesorbitan, monooleate, TWEEN®, SPAN® 20, SPAN® 40, SPAN® 60, SPAN® 80, IGEPAL®, Triton X-100, Neobee, lecithin, Pluronic 31R1, Pluronic 17R4, Brij 30 and a mixture thereof.

In certain exemplary embodiments, the hydrophobic payload is selected from the group consisting of a terpene, a terpenoid, eugenol, geraniol, thymol, clomazone, triallate, limonene, lambda-cyhalotrin, penthiopyrad (PTP), spinosad, tetrahydrocannabinol (THC), cannabinol, cannabidiol, cannabigerol (CBG), aminoglycoside antibiotics, gentamycin, kanamycin, macrolides, erythromycin, rifamycins, novobiocin, fusidic acid, cationic peptides, cycloserine, rifampicin, doxorubicin, epirubicin, idarubicin, mitoxantrone, aspirin, acetaminophen, d-propoxyphene, fenoprofen, flurbiprofen, ibuprofen, ketoprofen, naproxen, naproxen-anhydride, oxaprozin, small organic active agent, a small inorganic active agent, a microbicide, a fungicide, an insecticide, a nematicide, a pesticide, an antibiotic, an analgesic, a non-steroidal anti-inflammatory drug (NSAID), a chemotherapeutic, a dietary supplement, and a mixture thereof.

In certain exemplary embodiments, the hyperloaded YP has a diameter that is greater than the diameter of a non-hyperloaded YP. In certain exemplary embodiments, the diameter of the hyperloaded YP is between about 6 μm and about 10 μm.

### BRIEF DESCRIPTION OF THE DRAWINGS

The foregoing and other features and advantages of the present disclosure will be more fully understood from the following detailed description of illustrative embodiments taken in conjunction with the accompanying drawings. This patent or application file contains at least one drawing executed in color. Copies of this patent or patent application publication with color drawing(s) will be provided by the Office upon request and payment of the necessary fee.

FIG. 1A-FIG. 1B are schematic diagrams showing loading procedures to achieve standard 1:1 payload:YP ratio and high (up to 5:1) payload:YP ratio.

FIG. 2A-FIG. 2B depict (A) the kinetics of 2:1:2 GET loading in YPs quantified by HPLC of samples prepared at GET:YP ratio of 1.1:1 (final concentration of 150 g YP/L, 16.5% GET), and (B) light and fluorescent photomicrographs of Nile red stained YP control (t=0) and YPs loaded with GET collected at different timepoints showing Nile red stained terpenes encapsulated in YPs.

FIG. 3A-FIG. 3D depict light and fluorescent photomicrographs of empty YPs (control) and GET loaded YPs prepared at 1.1:1 to 5:1 GET:YP ratios showing encapsulated fluorescent terpene-Nile red complex for YPs for YPs obtained from Biorigin (A) and SAF Mannan (C) and the average particle size of empty YP control and GET loaded YPs for YPs obtained from Biorigin (B) and SAF Mannan (D).

FIG. 4A-FIG. 4F graphically depict the kinetics of dry-wet cycling terpene release. Cumulative GET release from Biorigin YPs showing extension of wetting/terpene release cycles in hyperloaded YP-GET samples: (A) GET:YP 1.1:1), (B) GET:YP 2:1, (C) GET:YP 3:1, (D) GET:YP 4:1, (E) GET:YP 5:1 and (F) time for 100% GET release.

FIG. 5 depicts light and fluorescent photomicrographs of YP (Biorigin)-GET424 at t=0 h and after complete release of GET.

FIG. 6A-FIG. 6F graphically depict the kinetics of dry-wet cycling terpene release. Cumulative GET release from SAF Mannan YPs showing extension of wetting/terpene release cycles in hyperloaded YP-GET samples: (A) GET:YP 1.1:1), (B) GET:YP 2:1, (C) GET:YP 3:1, (D) GET:YP 4:1 and (E) GET:YP 5:1 and number of cycles needed to achieve 100% GET release (F).

FIG. 7 depicts light and fluorescent photomicrographs of YP (SAF Mannan)-GET424 at t=0 h and after complete release of GET.

FIG. 8 depicts light and fluorescent photomicrographs of YP(Biorigin)-GET424 samples at t=0 h and after sonication once or five time for a duration of 30 seconds.

FIG. 9A-FIG. 9E depict the characteristics of YP-GET (10% YP, GET:YP ratio of 3.5:1) prepared in water or stabilized in 30% glycerin-70% water. (A) Percent GET encapsulation, (B) particle count, (C) average particle diameter and (D, E) light and fluorescent photomicrographs of samples after temperature stress (repeated freeze/thaw cycles and two-week storage at 23° C. and 54° C.).

FIG. 10A-FIG. 10C schematically depicts diffusion-controlled terpene loading in YPs at terpene:YP weight ratios of (A) 1.1:1 and (B) 5:1 showing an increase in yeast particle diameter for hyperloaded samples, and (C) schematics of terpene release from hyperloaded YPs and terpene re-loading and YP elasticity.

FIG. 11A-FIG. 11B (A) depicts light and fluorescent microscopy images of YP-GET 1:3 (10% YP, 30% GET) showing YPs remain intact following terpene loading, release, and terpene re-loading, and (B) average YP diameter.

FIG. 12A-FIG. 12C show (A) loading efficiency of limonene, (B) light and fluorescent microscopy images of YP-limonene loaded at limonene:YP w/w ratios of 1:1 and 3:1, and (C) average YP diameter measured along the major axis of the particles of YPs loaded at limonene ratios of 1:1 and 3:1.

FIG. 13 depicts clomazone loading conditions and encapsulation efficiency in YPs. CMZ=clomazone.

FIG. 14A-FIG. 14B show light and fluorescent microscopy images of two types of YPs loaded at clomazone:YP w/w ratios of 1:1 to 5:1 (A) and the average diameter of YPs loaded at various payload ratios (B).

FIG. 15 shows effect of storage temperature on YP-clomazone encapsulation stability. CMZ=clomazone.

FIG. 16A-FIG. 16C shows light and fluorescent microscopy images of YP(Biorigin)-clomazone loaded at clomazone:YP w/w ratios of 1:1 to 5:1 after three freeze/thaw cycles at −20° C. and 25° C. (A) and two week storage at 54° C. for two weeks (B) and the average diameter of YPs before and after temperature stress (C).

FIG. 17 shows triallate loading conditions and encapsulation efficiency in YPs. T=triallate.

FIG. 18A-FIG. 18B show light and fluorescent microscopy images of two type of YPs loaded at triallate:YP w/w ratios of 1:1 to 5:1 (A) and the average diameter of YPs loaded at various payload ratios (B).

FIG.19 shows effect of storage temperature on YP-triallate encapsulation stability. T=triallate.

FIG. 20A-FIG. 20B show light and fluorescent microscopy images of Biorigin YPs loaded at triallate:YP w/w ratios of 3:1 and 4:1 after temperature stress (repeated freeze/thaw cycles and two-week storage at 23° C. and 54° C.) (A) and the average diameter of YPs before and after temperature stress (B).

FIG. 21A-FIG. 21C show effect of shear stress on encapsulation stability of YP-triallate. (A) Triallate retained within YPs after sonication. (B) Light and fluorescent photomicrographs of YP(Biorigin)-triallate samples before and after one or multiple cycles of sonication. (C) Average particle diameter before and after sonication.

FIG. 22A-FIG. 22C show the kinetics of triallate release from Biorigin YPs upon dilution for YPs loaded at a w/w triallate:YP ratio of 3:1 (A) and 4:1 (B) and the percentage of triallate expected to be released at each dilution (C).

FIG. 23A-FIG. 23C show tetrahydrocannabinol (THC) loading conditions and encapsulation efficiency in GLPs (A), light and fluorescent microscopy images of GLPs loaded at THC:GLP w/w ratios of 1:1 to 5:1 (B) and the average diameter of GLPs loaded at various payload ratios (C).

FIG. 24 shows λ-cyhalothrin (λCy) loading conditions and encapsulation efficiency in YPs.

FIG. 25A-FIG. 25B show light and fluorescent microscopy images of GLPs loaded at λCy:GLP w/w ratios of 1:1 to 5:1 (A) and the average diameter of GLPs loaded at various payload ratios (B).

FIG. 26A-FIG. 26B show the kinetics of λCy release from YPs upon dilution for YPs loaded at a w/w λCy:YP ratio of 1:1 (A) and the percentage of λCy expected to be released at each dilution (B).

FIG. 27A-FIG. 27B are schematic diagrams showing procedures using organic solvent to load payloads are water-insoluble or have low water solubility. Solvent may be completely removed after loading is completed (A) or may remain inside YP as “leave-in” solvent along with payload (B).

FIG. 28A-FIG. 28C show penthiopyrad (PTP) loading conditions, encapsulation efficiency, and number of loading cycles required to achieve high payload:YP weight ratios (A), light and fluorescent microscopy images of YPs loaded at PTP:YP w/w ratios of 1:1 to 4:1 (B) and the average diameter of YPs loaded at various payload ratios (C).

FIG. 29A-FIG. 29C show cannabidiol (CBD) loading conditions, encapsulation efficiency, and number of loading cycles required to achieve high payload:GLP weight ratios (A), light and fluorescent microscopy images of GLPs loaded at PTP:GLP w/w ratios of 1:1 to 5:1 (B) and the average diameter of GLPs loaded at various payload ratios (C).

FIG. 30A-FIG. 30C show spinosad (S) loading conditions and encapsulation efficiency with the use of GET424 as a leave-in solvent (A), light and fluorescent microscopy images of YPs loaded at (Spinosad+GET424):YP w/w ratios of 1:1 (B) and the average diameter of YPs loaded with payload and leave-in solvent (C).

FIG. 31 shows the kinetics of spinosad release from YPs upon dilution.

FIG. 32 shows penthiopyrad loading conditions and encapsulation efficiency in YPs using leave-in solvent GET424.

FIG. 33A-FIG. 33B show light and fluorescent microscopy images of YPs loaded at PTP:YP w/w ratios of 0.5:1 to 2.5:1 (A) and the average diameter of YPs loaded at various payload ratios (B).

FIG. 34A-FIG. 34C show penthiopyrad loading conditions and encapsulation efficiency in YPs using leave-in solvent DMDA (A), light and fluorescent microscopy images of YPs loaded at PTP:YP w/w ratios of 0.5:1 to 2.5:1 (B) and the average diameter of YPs loaded at various payload ratios (C).

FIG. 35A-FIG. 35D show the kinetics of PTP release from YPs upon dilution. Release of YPs containing PTP-YPs without leave-in solvent (A), with GET424 as a leave in solvent (B), with DMDA as a leave-in solvent (C) is shown along the percentage of PTP expected to be released at each dilution (D).

FIG. 36A-FIG. 36C show cannabidiol (CBD) loading conditions and encapsulation efficiency in GLPs using leave-in solvent octanoic acid (OA) (A), light and fluorescent microscopy images of GLPs loaded at (CBD-0A):GLP w/w ratios of 3:1 (B), and the average diameter of empty GLPs and GLPs loaded with CBD-OA (C).

FIG. 37A-FIG. 37B show the results of spectrophotometric quantification of encapsulated fish oil (A) and fluorescent photomicrographs of Nile Red stained YPs (B).

FIG. 38A-FIG. 38B show prothioconazole (PRO) loading conditions, encapsulation efficiency, and number of loading cycles required to achieve high payload:YP weight ratios (A), light and fluorescent microscopy images of YPs loaded at PRO:YP w/w ratios of 1:1 to 3:1 (B).

### DETAILED DESCRIPTION

The present disclosure improves upon conventional encapsulation technologies by providing a yeast particle (YP) delivery system comprising an extracted yeast cell wall and a hydrophobic payload.

In the present disclosure, hydrophobic payload molecules are loaded into the YPs to make hyperloaded YPs, e.g., at a payload : YP weight/weight ratio from 2:1 up to 5:1 such that the chemical or biologic activities of the payloads are not permanently altered or diminished. The methods of the present disclosure can achieve a greater loading capacity, increased temperature stability and sustained release of the payload from the hyperloaded YP, thereby, providing for a significant improvement over existing technologies.

The disclosures of patents, patent applications, and publications in their entireties are hereby incorporated by reference into this application in order to more fully describe the state of the art as known to those skilled therein as of the date of the invention described and claimed herein (e.g., U.S. Pat. Nos. 9,655,360, 10,004,229, European Patent No. 1711058, WO2005070213A2, WO2005113128A1 and associated patents/patent applications). The instant disclosure will govern in the instance that there is any inconsistency between the patents, patent applications, and publications and this disclosure.

That the disclosure may be more readily understood, select terms are defined below.

### Definitions

### Yeast Particles

As used herein, a “yeast particle” (YP) refers to readily available, biodegradable, substantially spherical, hollow particles of about 2-4 μm in diameter. YPs may be obtained as a byproduct of some food grade Baker's yeast (i.e., Saccharomyces cerevisiae) extract manufacturing processes. YPs include, but are not limited to, commercially available YPs (for example, Biorigin™ and SAFMANNAN™), extracted yeast cell wall particles (YCWPs), yeast cell particles (YCPs), glucan particles (GPs), yeast glucan particles (YGPs), yeast glucan-mannan particle (YGMP), glucan lipid particles (GLPs), whole glucan particles (WGPs) and the like. Methods of preparing extracted yeast cell wall particles are known in the art, and are described, for example in U.S. Pat. Nos. 4,992,540, 5,028,703, 5,032,401, 5,322,841, 5,401,727, 5,504,079, 5,968,811, 6,444,448, 6,476,003, published U.S. applications 2003/0216346 A1, 2004/0014715 A1, and PCT published application WO 02/12348 A2, which are specifically incorporated herein by reference.

### Hydration of Yeast Particles

A sufficient level of hydration of YPs is needed for encapsulation and release of payloads. For example, encapsulation of limonene powder does not work unless some water is present (Errenst, C.; Petermann, M.; Kilzer, A. Encapsulation of limonene in yeast cells using the concentrated powder form technology. J. Supercrit. Fluid 2021, 168, 105076). Dardelle et al. demonstrated that a minimum of 20% hydration is necessary for limonene release (Dardelle, G.; Normand, V.; Steenhoudt, M.; Bouquerand, P.-E.; Chevalier, M.; Baumgartner, P. Flavour-Encapsulation and flavour-release performances of a commercial yeast-based delivery system. Food Hydrocoll. 2007, 21, 953-960.). Dimopoulous et al. also highlighted the need for water activity (aw)>0.7 to obtain release (Dimopoulos, G.; Katsimichas, A.; Tsimogiannis, D.; Oreopoulou, V.; Taoukis, P. Cell permeabilization processes for improved encapsulation of oregano essential oil in yeast cells. J. Food Eng. 2021, 294, 110408).

Dry YPs can be hydrated by incubation with a variety of aqueous solutions. Suitable aqueous solutions include, but are not limited to: water; saline, e.g., phosphate buffered saline; any buffer solution known in the art with a pH between 3 and 11; any acid solution known in the art with a pH>1.5; any basic solution known in the art with a pH<11; any salt solution known in the art that does not chemically interfere with the payload, and the like.

### Payload Molecules

The hyperloaded YPs of the present disclosure are useful for in vivo or in vitro delivery of payload molecules to a cell or an organism. Hyperloaded YPs are useful for the delivery of hydrophobic, water-insoluble molecular payloads that cannot be encapsulated at high payload: YP w/w ratios within yeast particles using any art-known method.

The term “hydrophobic payload” as used herein refers to molecules or substituents that are non-polar, have little or no affinity for water, and tend to repel water. In certain embodiments, hydrophobic payloads are compounds which are inherently hydrophobic, for example having a having a log P of at least 2 (Log P is the log of the octanol-water or buffer partition coefficient and can be determined by a variety of methods for those skilled in the art. The higher the value of log P, the greater the hydrophobicity of the chemical.) Any molecular payload that is a water-insoluble payload is envisioned by the present disclosure. In certain embodiments the hydrophobic payload is selected from the group consisting of a terpene, a terpenoid, eugenol, geraniol, thymol, clomazone, triallate, limonene, lambda-cyhalotrin, penthiopyrad (PTP), prothioconazole (PRO), spinosad, tetrahydrocannabinol (THC), cannabinol, cannabidiol, cannabigerol (CBG), fish oil, aminoglycoside antibiotics, gentamycin, kanamycin, macrolides, erythromycin, rifamycins, novobiocin, fusidic acid, cationic peptides, cycloserine, rifampicin, doxorubicin, epirubicin, idarubicin, mitoxantrone, aspirin, acetaminophen, d-propoxyphene, fenoprofen, flurbiprofen, ibuprofen, ketoprofen, naproxen, naproxen-anhydride, oxaprozin, small organic active agent, a small inorganic active agent, a microbicide, a fungicide, an insecticide, a nematicide, a pesticide, an antibiotic, an analgesic, a non-steroidal anti-inflammatory drug (NSAID), a chemotherapeutic, a dietary supplement, and any mixtures thereof.

### A. Terpene Payloads

In certain embodiments, the disclosure provides compositions and methods for the encapsulation and delivery of one or more terpene payload molecules. Any terpene payload may be hyperloaded, encapsulated, and delivered to a subject according to the methods of the present disclosure. The terpene payload may comprise a single terpene or a mixture of terpenes.

The term “terpene” or “terpene payload” as used herein refers to both a terpene of formula (C₅H₈)ₙ, and a terpene derivative, such as a terpene aldehyde. In addition, reference to a single name of a compound will encompass the various isomers of that compound. For example, the term citral includes the cis-isomer citral-a (or geranial) and the trans-isomer citral-b (or neral).

As used herein, terpenes refer to chemical compounds that are widespread in nature, mainly in plants as constituents of essential oils. Their building block is the hydrocarbon isoprene (C₅H₈)ₙ. Examples of terpenes include, but are not limited to, citral, pinene, nerol, b-ionone, geraniol, carvacrol, eugenol, carvone, terpeniol, anethole, camphor, menthol, limonene, nerolidol, framesol, phytol, carotene (vitamin Al), squalene, thymol, tocotrienol, penny' alcohol, borneol, myrcene, simene, carene, terpenene, and linalool. A mixture of geraniol (G), eugenol (E), and thymol (T) can also be used wherein the G:E:T weight ratio is about 1:1:1, 1:1:2, 1:2:1, 1:2:2, 2:1:1, 2:1:2, 2:2:1, 2:2:2 or multiples thereof. The mixture is referred to as “GET”. The terms “GET212” and “GET424” refer to a mixture of geraniol, eugenol and thymol at a weight ratio of about 2:1:2., 2.1:1:2, 2.1:1.1:2, 2.1:1.1:2.1, 1.9:1:2, 1.9:0.9:2, or 1.9:0.9:1.9.

Terpenes are classified as generally recognized as safe (GRAS) and have been used for many years in the flavoring and aroma industries. The list of terpenes which are exempted from US regulations found in EPA regulation 40 C.F.R. Part 152 is incorporated herein by reference in its entirety. Terpenes have a relatively short life span of approximately 28 days once exposed to oxygen (e.g., air). Terpenes decompose to CO2, further demonstrating the safety and environmental friendliness of the compositions and methods of the disclosure.

Terpenes have been found to inhibit the in vitro growth of bacteria and fungi (Chaumont et al., Ann. Pharm. Fr., 1992, 50(3): 156-166; Moleyar et al., Int. J. Food Microbiol., 1992, 16(4): 337-342; and Pattnaik et al., Microbios., 1997, 89(358): 39-46) and some internal and external parasites (Hooser et al., J. Am. Vet. Med. Assoc., 1986, 189(8): 905-908). The terpene geraniol is the active component (75%) of rose oil. Rose oil and geraniol at a concentration of 2 mg/L inhibited the in vitro growth of H. pylori. Geraniol was found to inhibit the growth of C. albicans and S. cerevisiae strains by enhancing the rate of potassium leakage and disrupting membrane fluidity (Bard et al., Lipids, 1998, 23(6): 534-538).

There may be different modes of action of terpenes against microorganisms: they (1) interfere with the phospholipid bilayer of the cell membrane, (2) impair a variety of enzyme systems (HMG-reductase), and (3) destroy or inactivate genetic material. Without intending to be bound by scientific theory, it is believed that due to the modes of action of terpenes being so basic, e.g., blocking of cholesterol, that infective agents do not build a resistance to terpenes.

The terpenes and other components of the pre-payloads according to the disclosure may be readily purchased or synthesized using techniques generally known to synthetic chemists. Useful terpenes according to the present disclosure, for safety and regulatory reasons, are at least food grade terpenes, as defined by the United States FDA or equivalent national regulatory body outside the USA.

Alternatively, stable terpene solutions can be obtained by mixing terpenes and water at high shear. See PCT Patent Application Publication WO2003/020024. Regardless of how they are prepared, terpenes are prone to oxidation in aqueous emulsion systems, which make long term storage a problem. Thus, the composition of the present disclosure can comprise an antioxidant to reduce oxidation of the terpene. A non-limiting example of such an anti-oxidant might be rosemary oil, vitamin C, or vitamin E. Preservatives and other additives can also be present such as, for example, antimicrobials, anti-oxidants, chelating agents, and inert gases.

Terpenes can be taken up and stably encapsulated within hollow glucan particles or cell wall particles. See United States Patent U.S. Pat. No. 9,439,416, the contents of which are incorporated by reference in its entirety. Encapsulation of terpenes into such particles can be achieved by incubation of the particles with the terpene. Nevertheless, terpenes rapidly diffuse from the glucan shell when encapsulated according to conventional methods. Accordingly, certain exemplary embodiments of the present disclosure provide for improved compositions and methods for the encapsulation and delivery of terpenes.

Compositions of the present disclosure can comprise other active compounds, alone or in addition to a terpene component. The compositions can comprise a further active agent in addition to the terpene component, for example, an antimicrobial agent, an anti-fungal agent, an insecticidal agent, an anti-inflammatory agent, an anesthetic, or the like.

Suitable agents include, but are not limited to, antifungals, such as cell wall hydrolases, to the extent they do not degrade the hollow glucan particle or cell wall particle, cell wall synthesis inhibitors, and standard antifungals; antibacterials, such as antiseptics, cell wall hydrolases, synthesis inhibitors, and antibiotics; and insecticides, such as natural insecticides and chitinase.

### B. Antimicrobial Payloads

Certain exemplary embodiments of the present disclosure provide for compositions and methods for the loading and delivery of hydrophobic payload molecules with antimicrobial activity effective against classes of organisms such as Gram positive bacteria, Gram negative bacteria, fungi, and viruses.

As used herein, the term “antimicrobial” refers to the ability of a compound to inhibit or irreversibly prevent the growth of a microorganism. Such inhibition or prevention can be through a microbicidal action or microbistatic inhibition. The term “microbicidal inhibition” refers to the ability of the antimicrobial compound to kill, or irrevocably damage the target organism. The term “microbistatic inhibition” as used herein refers to the ability of the antimicrobial compound to inhibit the growth of the target organism without death.

A compound with microbicidal or microbistatic inhibitory properties can be applied to an environment either presently exhibiting microbial growth (i.e., therapeutic treatment) or to an environment at risk of supporting such growth (i.e., prevention or prophylaxis). An environment capable of sustaining microbial growth refers to a fluid, substance, or organism where microbial growth can occur or where microbes can exist. Such environments can be, for example, animal tissue or bodily fluids, water and other liquids, food, food products or food extracts, crops, and certain inanimate objects. It is not necessary that the environment promote the growth of the microbe, only that it permit its subsistence.

Any suitable hydrophobic antimicrobial compound may be encapsulated according to the methods presently described. In certain nonlimiting embodiments, the antimicrobial compound is an antibiotic, such as aminoglycosides (gentamycin, kanamycin), macrolides (erythromycin), rifamycins, novobiocin, fusidic acid, cationic peptides, cycloserine, and rifampicin. The hydrophobic antimicrobial payload component may comprise a single microbial or a mixture of antimicrobials.

### C. Chemotherapeutic Payloads

Certain exemplary embodiments of the present disclosure also provide compositions and methods for the encapsulation and delivery of hydrophobic payload molecules with chemotherapeutic or anticancer properties. Any solid or hematological cancer may be treated with the hydrophobic payload molecules presently disclosed.

Exemplary useful chemotherapeutic agents include alkylating agents, anti-metabolites, alkaloids, and miscellaneous agents (including hormones), and certain antibiotics. For example, anthracyclines are one of the more commonly used chemotherapeutic antibiotics. Anthracycline antibiotics are produced by the fungus Streptomyces peuceitius var. caesius. Anthracycline antibiotics have tetracycline ring structures with an unusual sugar, daunosamine, attached by glycosidic linkage. Cytotoxic agents of this class all have quinone and hydroquinone moieties on adjacent rings that permit them to function as electron-accepting and donating agents.

Anthracyclines achieve their cytotoxic effect by several mechanisms, including intercalation between DNA strands, thereby interfering with DNA and RNA synthesis; production of free radicals that react with and damage intracellular proteins and nucleic acids; chelation of divalent cations; and reaction with cell membranes. The wide range of potential sites of action may account for the broad efficacy as well as the toxicity of the anthracyclines.

Any suitable hydrophobic chemotherapeutic or antitumor compound may be encapsulated according to the methods presently described. In certain embodiments, the chemotherapeutic or antitumor compound is selected from the group consisting of doxorubicin, epirubicin, idarubicin, and mitoxantrone. The chemotherapeutic or anticancer payload component may comprise a single payload molecule or a mixture of payload molecules.

### D. Non-Steroidal Anti-Inflammatory Drug (NSAID) Payloads

The disclosure also provides compositions and methods for the encapsulation and delivery of payload molecules with analgesic and anti-inflammatory properties. The analgesic or anti-inflammatory payload component may comprise a single pro-payload molecule or a mixture of payload molecules. Any useful analgesic or anti-inflammatory compound may be encapsulated according to the methods presently described.

In certain embodiments, the analgesic or anti-inflammatory compound is selected from the group consisting of aspirin, acetaminophen, d-propoxyphene, fenoprofen, flurbiprofen, ibuprofen, ketoprofen, naproxen, and oxaprozin.

Nonsteroidal anti-inflammatory drugs (NSAIDs) are a drug class that reduce pain, decrease fever, prevent blood clots and, in higher doses, decrease inflammation. Useful NSAIDs include, without limitation, aspirin, ibuprofen and naproxen.

Naproxen is a well-known NSAID, with a daily dose ranging from about 250 to about 1500 milligrams, or from about 500 to about 1000 milligrams. Naproxen, and other analgesic drugs, can be administered in multiple doses over 12 or 24 hours.

Additionally, a higher initial dose, followed by relatively low maintenance doses, can be delivered. See, e.g., Palmisano et al., Advances in Therapy, Vol. 5, No. 4, July/August 1988; describing the use of multiple doses of ketoprofen (initial dose of 150 mg followed by subsequent doses of 75 mg) and ibuprofen (initial dose of 800 mg followed by subsequent doses of 400 mg).

Controlled release pharmaceutical dosage forms can be used to optimize drug delivery and enhance patient compliance. A pharmaceutical dosage form can deliver more than one drug, each at a modified rate.

### Hyperloaded YPs

As used herein, a “hyperloaded YP” refers to a YP that has been loaded with payload at a high capacity such that the ratio of weight of the payload to the weight of the YP (weight/weight ratio, payload:YP) is equal to or greater than 1:1. In certain embodiments, the weight by weight (w/w) ratio of payload:YP can range from about : >1 to about 10:1, from about 1.5:1 to about 7.5:1, or from about 2.0:1 to about 5:1. For example, the ratio of payload:YP can be about 1.0:1, about 1.1:1, about 1.2:1, about 1.3:1, about 1.4:1, about 1.5:1, about 1.6:1, about 1.7:1, about 1.8:1, about 1.9:1, about 2.0: 1, about 2.1:1, about 2.2:1, about 2.3:1, about 2.4:1, about 2.5:1, about 2.6:1, about 2.7:1, about 2.8:1, about 2.9:1, about 3.0:1, about 3.1:1, 3.2:1, 3.3:1, about 3.4:1, 3.5:1, 3.6:1, 3.7:1, 3.8:1, 3.9:1, about 4:1, about 4.1:1, about 4.2:1, about 4.3:1, about 4.4:1, about 4.5:1, about 4.6:1, about 4.7:1, about 4.8:1, about 4.9:1, about 5:1, about 5.1:1, about 5.2:1, about 5.3:1, about 5.4:1, about 5.5:1, about 5.6:1, about 5.7:1, about 5.8:1, about 5.9:1, about 6.1:1, 6.2:1, 6.3:1, about 6.4:1, about 6.5:1, about 6.6:1, about 6.7:1, about 6.8:1, about 6.9:1, about 7.1:1, about 7.2:1, about 7.3:1, about 7.4:1, about 7.5:1, about 7.6:1, about 7.7:1, about 7.8:1, about 7.9:1, about 8.1:1, about 8.2:1, about 8.3:1, about 8.4:1, about 8.5:1, about 8.6:1, about 8.7:1, about 8.8:1, about 8.9:1, about 9.1:1, about 9.2:1, about 9.3:1, about 9.4:1, about 9.5:1, about 9.6:1, about 9.7:1, about 9.8:1, about 9.9:1 or about 10:1.

As used herein, a “non-hyperloaded YP” refers to a YP that has been loaded with a payload such that the ratio of the weight of the payload to the weight of the YP (weight/weight ratio; payload:YP) is 1:1 or <1:1.

Diameter of the hyperloaded and non-hyperloaded YPs is measured after payload loading is complete. Hyperloaded YPs have an average diameter that is larger than non-hyperloaded YPs. In certain exemplary embodiments, non-hyperloaded YPs have a diameter of about 2-5 μm. For example, a hyperloaded YP can have a diameter of about 5 μm, about 5.5 μm, about 6μm, about 6.5 μm, about 7 μm, about 7.5 μm, about 8 μm, about 8.5 μm, about 9 μm, about 9.5 μm, or about 10 μm. In certain embodiments, the diameter of a hyperloaded YP is between about 5 μm and about 10 μm, between about 6 μm and about 9 μm or between about between about 6 μm and about 8 μm.

### Payload Release from YPs

As used here in the term “release” of payload refers to the diffusion of loaded payload from interior of the YP to the exterior. In certain exemplary embodiments, payloads in hyperloaded YPs are released upon contact with an aqueous solution. As used herein an “aqueous solution” refers to water or an aqueous buffer.

### Solvents, Loading Solvent, and Leave-in Solvents

Solvents may be added during the encapsulation process to facilitate loading of payloads in the YPs. Certain payloads of the present disclosure are water-insoluble or have low water solubility and may be loaded into YPs with a solvent that is compatible with yeast particles. In certain nonlimiting embodiments, the solvent may be an organic solvent. Suitable solvents include, but are not limited to, be acetone, dichloromethane, ethyl acetate, alcohols such as ethanol or methanol, dimethylsulfoxide (DMSO), methanol-chloroform, hexane, petroleum ether, toluene, Neobee and the like. After a payload is completely encapsulated, the yeast particle and payloads may be processed to remove the solvent from the YP-payload formulation. Organic solvents such as acetone, dichloromethane, ethyl acetate, methanol, and DMSO may be unsafe for human administration and should be removed after a payload is completely encapsulated. Alternatively, the solvent used to facilitate payload encapsulation may be safe for human administration and can be left inside the YP along with the hydrophobic payload as a “leave-in solvent.”

In certain embodiments, an organic solvent is selected from the group consisting of acetone, dichloromethane, ethyl acetate, ethanol, methanol, dimethyl sulfoxide (DMSO), eugenol, geraniol, a mixture of geraniol (G), eugenol (E), thymol (T) at a weight ratio composition of 2:1:2 (GET424), N,N-dimethyl-decanamide (DMDA), octanoic acid, lauric acid, undecanoic acid, glycofurol, vitamin E, fatty acid, medium chain triglycerides (MCT), and a mixture thereof.

In certain embodiments, a leave-in solvent is a mixture of geraniol (G), eugenol (E), thymol (T) at a weight ratio composition of about 2:1:2 (GET424), N,N-dimethyl-decanamide (DMDA), octanoic acid, or a mixture thereof.

### Surfactants

The term “surfactant,” as used herein, refers to any molecule having both a hydrophilic group (e.g., a polar group), which energetically prefers solvation by water, and a hydrophobic group which is not well solvated by water. The term “nonionic surfactant” is a known term in the art and generally refers to a surfactant molecule whose hydrophilic group (e.g., polar group) is not electrostatically charged.

Surfactants are generally low to moderate weight compounds which contain a hydrophobic portion, which is generally readily soluble in oil, but sparingly soluble or insoluble in water, and a hydrophilic portion, which is sparingly soluble or insoluble in oil, but readily soluble in water. In addition to protecting against growth and aggregation and stabilizing the organic compound delivery vehicle, surfactants are also useful as excipients in organic compound delivery systems and formulations because they increase the effective solubility of an otherwise poorly soluble or non-soluble organic compound, and may decrease hydrolytic degradation, decrease toxicity and generally improve bioavailability. Surfactants may also provide selected and advantageous effects on drug release rate and selectivity of drug uptake. Surfactants are generally classified as either anionic, cationic, or nonionic.

Suitable surfactants include, but are not limited to, sodium lauryl sulphate, polysorbate 20, polysorbate 80, polysorbate 40, polysorbate 60, polyglyceryl ester, mono fatty acid ester of polyoxyethylene sorbitan, polyglyceryl monooleate, decaglyceryl monocaprylate, propylene glycol dicaprilate, poly ethylenepolypropylene glycol, triglycerol monostearate, polyoxyethylenesorbitan, monooleate, Tween®, Span® 20, Span® 40, Span® 60, Span® 80, IGEPAL®, Triton X-100, Neobee Brij 30 and the like, and any mixtures thereof.

### Temperature Stabilizing Agents

As used herein the term “temperature stabilizing agent” refers to a chemical that improves the storage stability of YPs containing payloads. Common temperature stabilizing agents include sugars such sucrose, trehalose, glycerol, or sorbitol. Disaccharides such as sucrose and trehalose are natural cryoprotectants with good protective properties. A temperature stabilizing agent may comprise a carbohydrate component including between about 10% and 80% oligosaccharide, between about 5% and 30% disaccharide or between about 1% and 10% polysaccharide, and a protein component including between about 0.5% and 40% protein, e.g., hydrolyzed animal or plant proteins, based on the total weight of the composition. Ascorbic acid ions may be used in some embodiments for stabilization at higher temperature and humidity exposure. Alternatively, a combination of citrate and/or ascorbate ions with protein or protein hydrolysate may be used. In certain nonlimiting embodiments, the temperature stabilizing agent may be a glycerin. In certain nonlimiting embodiment temperature stabilizing agent may be glycerin at a concentration of about 5%, about 10%, about 15%, about 20%, about 25%, about 30% , about 35%, about 40%, about 45% or about 50%. In certain nonlimiting embodiment temperature stabilizing agent may be glycerin at a concentration of 1-5%, 5-10%, 10-15%, 15-20%, 20-25%, 25-30%, 35-40%, 40-45%, or 45%-50%.

### General Definitions

Standard techniques may be used for recombinant DNA, oligonucleotide synthesis, and tissue culture and transformation (e.g., electroporation, lipofection). Enzymatic reactions and purification techniques may be performed according to manufacturer's specifications or as commonly accomplished in the art or as described herein. These and related techniques and procedures may be generally performed according to conventional methods well known in the art and as described in various general and more specific references that are cited and discussed throughout the present specification. Unless specific definitions are provided, the nomenclature utilized in connection with, and the laboratory procedures and techniques of, molecular biology, analytical chemistry, synthetic organic chemistry, and medicinal and pharmaceutical chemistry described herein are those well-known and commonly used in the art. Standard techniques may be used for recombinant technology, molecular biological, microbiological, chemical syntheses, chemical analyses, pharmaceutical preparation, formulation, and delivery, and treatment of patients.

As used in this specification and the appended claims, the singular forms “a,” “an” and “the” include plural references unless the content clearly dictates otherwise. Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers. Each embodiment in this specification is to be applied mutatis mutandis to every other embodiment unless expressly stated otherwise.

As used herein, the term “about” in quantitative terms refers to plus or minus 5% of the value it modifies (rounded up to the nearest whole number if the value is not subdividable, such as a number of molecules, nucleotides, or amino acids). For example, “about 20%” would encompass 15-20% and “about 80%” would encompass 75-85%, inclusive. Moreover, where “about” is used herein in conjunction with a quantitative term it is understood that in addition to the value plus or minus 5%, the exact value of the quantitative term is also contemplated and described. For example, the term “about 23%” expressly contemplates, describes, and includes exactly 23%.

### Articles of Manufacture, Compositions, and Methods

In another aspect, the present disclosure provides an article of manufacture or kit comprising a first container containing a hydrophobic payload molecule, wherein the payload molecule is selected from the group consisting of a small organic active agent, a small inorganic active agent, a microbicide, a fungicide, an insecticide, a nematocide, a pesticide, an antibiotic, an analgesic, a non-steroidal anti-inflammatory drug (NSAID), a terpene, a terpenoid, a tetrahydrocannabinol, a cannabidiol, a chemotherapeutic, a dietary supplement, and mixtures thereof, a second container containing hyperloaded YPs comprising a yeast cell wall particle, and instructions for use.

In another aspect, the present disclosure provides methods of making hyperloaded YPs comprising the steps of providing an extracted yeast cell wall comprising beta-glucan, the yeast cell wall defining an internal space; incubating the hydrophobic payload with the yeast particle in the presence or absence of a solvent, wherein the hydrophobic payload molecule becomes enclosed within the internal space, thereby forming hyperloaded YPs.

In another aspect, the present disclosure provides a pharmaceutical composition comprising hyperloaded YPs comprising a yeast cell wall particle, a hydrophobic payload molecule, wherein the payload molecule is selected from the group consisting of a polynucleotide, a polynucleotide, a peptide, a protein, a small organic active agent, a small inorganic active agent, a microbicide, a fungicide, an insecticide, a nematocide, a pesticide, an antibiotic, an analgesic, a non-steroidal anti-inflammatory drug (NSAID), a terpene, a terpenoid, a tetrahydrocannabinol, a cannabidiol, a chemotherapeutic, a dietary supplement, and mixtures thereof, and a pharmaceutically acceptable excipient.

In another aspect, the present disclosure provides methods of using hyperloaded YPs. In certain embodiments, the disclosure provides a method of delivering a payload molecule of the present disclosure to a cell, comprising: (a) incubating a hydrophobic payload molecule with a yeast cell wall particle defining an internal space and comprising beta glucan, wherein the payload molecule becomes at least partially enclosed within the internal space, thereby forming hyperloaded YPs; and (b) contacting a cell with the particulate delivery system under conditions that permit internalization of the particulate delivery system and release and delivery of the payload molecule within the cell.

### Agricultural and Industrial Compositions and Methods

The compositions and methods of the present disclosure are useful in the fields of consumer and industrial products, e.g., in food, human and animal drugs, cosmetics, and agriculture. In some embodiments, the compositions and methods of the present disclosure extend to agricultural applications. In certain embodiments, the present disclosure relates to the development and delivery of stable and controlled-release microbiocides, fungicides, insecticides, nematocides, and pesticides to agricultural species, e.g., plants and/or animals.

Some embodiments of the present disclosure provide compositions and methods useful in the control of a variety of agricultural pests. As used herein, the term “pest” refers to organisms that negatively affect a host, e.g., a plant or an animal host (e.g., a mammalian host) by colonizing, damaging, attacking, competing with them for nutrients, or infecting them. Pests include, e.g., microbes, fungi, weeds, nematodes, and arthropods. Arthropods include insects and arachnids, as well as sucking and biting pests such as mites, ticks, ants, and lice.

Certain embodiments of the present disclosure provide compositions and methods for use in controlling sucking and biting pests, including e.g., mosquitoes, ticks, lice, fleas, mites, flies, and spiders.

Certain embodiments of the present disclosure provide for compositions and methods for use in controlling nematodes. Nematodes (Kingdom: Animalia; Phylum: Nematoda) are microscopic round worms. They can generally be described as aquatic, triploblastic, unsegmented, bilaterally symmetrical roundworms, that are colorless, transparent, usually bisexual, and worm-shaped (vermiform), although some can become swollen (pyroform).

Many nematodes are obligate parasites and a number of species constitute a significant problem in agriculture. Thus, methods to control their parasitic activities are an important feature in maximizing crop production in modern intensive agriculture.

Nematodes are not just parasitic to plants but a number of species are parasitic to animals, both vertebrate and invertebrate. Around 50 species attack humans and these include Hookworm (Anclyostoma), Strongylids (Strongylus), Pinworm (Enterolobius), Trichinosis (Trichina), Elephantitis (Wuchereria), Heartworm (Dirofilaria), and Ascarids (Ascaris).

In some embodiments of the present disclosure, any of the compositions described above may be formulated in a deliverable form suited to a particular application. Deliverable forms that can be used in accordance with embodiments of the present disclosure include, but are not limited to, liquids, emulsions, emulsifiable concentrates, solids, aqueous suspensions, oily dispersions, pastes, granules, powders, dusts, fumigants, and aerosol sprays. Suitable deliverable forms can be selected and formulated by those skilled in the art using methods currently known in the art. The compositions can be provided in combination with an agriculturally, food, or pharmaceutically acceptable carrier or excipient in a liquid, solid, or gel-like form. For solid compositions, suitable carriers include pharmaceutical or food grades of mannitol, lactose, starch, magnesium stearate, sodium saccharin, talc, cellulose, glucose, sucrose, and magnesium carbonate. Suitably, the formulation is in tablet or pellet form. As suitable carrier could also be a human or animal food material. Additionally, conventional agricultural carriers could also be used.

The use of terpenes to prevent and treat infections of plants by bacteria, phytoplasmas, mycoplasmas, or fungi are disclosed in PCT patent application publication WO2003/020024, which is incorporated by reference herein. Accordingly, the present disclosure further provides the use of any of the above compositions in the treatment or prevention of a plant infection.

Other plant infection that may be treated or prevented in accordance with the present disclosure may be caused by one or more of the following: Aspergillius fumigatus, Sclerotinia homeocarpa, Rhizoctonia solani, Colletotrichum graminicola, Phytophtora infestans, or Penicillium sp. As described herein, terpenes and/or the other therapeutic molecules, alone in suspension or solution may be somewhat unstable and may degrade rapidly in the soil environment, thus losing efficacy. Incorporation of a terpene or other therapeutic component in a hollow glucan particle or cell wall particle reduces the rate of release and degradation, thus increasing the duration of action of the molecule in the soil or on the plant. Accordingly, the terpene pro-payload and other components may be encapsulated as detailed above.

An advantage of a terpene based treatment of plants is that it can be applied shortly before harvest. Many conventional treatments require an extended period before re-entry to the treated area (generally 3 weeks). This means that an outbreak of a plant disease shortly before harvest cannot be treated with conventional treatments as it would then not be possible to harvest the crop at the desired time. The compositions of the present disclosure can suitably be applied at any time up until harvest, for example 21 days prior to harvest, 14 days prior to harvest, 7 days prior to harvest, or even 3 days or less before harvest. Prevention of plant infections can be achieved by treating plants which the compositions of the present disclosure regularly as a prophylactic measure.

Suitably, the composition of the present disclosure is applied by spraying. This is suitable for treating a plant disease which affects the surface of a plant. For spraying, a preparation comprising 2 g/l of the composition in water may be used. Concentrations of from 2 to 4 g/l are effective, and concentrations of greater than 4 g/l can be used as required. Obviously, it is important that the concentration of the composition used is sufficient to kill or inhibit the disease-causing agent, but not so high as to harm the plant being treated.

When spraying plants, a rate of 100 L/Ha or higher may generally be suitable to cover the plant. Typically, a rate of 100 to 500 L/Ha may be sufficient for crops of small plants which do not have extensive foliage; though higher rates may of course also be used as required. For larger plants with extensive foliage (e.g. perennial crop plants such as vines or other fruit plants) rates of 500 L/Ha or greater are generally suitable to cover the plants. A rate of 900 L/Ha or greater or 1200 L/Ha or greater is used to ensure good coverage. Where grape vines are being treated, a rate of 1200 L/Ha has proven suitably effective.

The composition of the present disclosure may alternatively be applied via irrigation. This is suitable for treating nematodes or other soil borne pathogens or parasites.

In certain embodiments, the present disclosure provides for compositions in the form of granules and methods of controlling pests using the same. Granules allow for the use of less selective herbicides, pesticides, and combinations thereof, and thus offer a means to control pests that are not otherwise easily controlled. Granules are a convenient application form for producers with small allotments such as paddy rice farmers, or for growers of turf where spays are complicated by the needs of near neighbors sensitive to drift or odor or for broad acre farmers who wish to apply fertilizers and herbicides together and who do not have convenient access to water.

The granules may be used in flooded paddies, recently irrigated turf, or in areas where it is inconvenient or impossible to remove irrigation water. The granules allow small holders the means to apply crop protection chemicals without expensive equipment, and without risk of exposing airways or eyes to aerosols or spray materials. Granules can be easily measured and distributed by hand. Using granules that are designed for uniform dispersal is advantageous because this compensates for uneven application.

### Pharmaceutical Compositions and Administration

In addition, the compositions and methods of the present disclosure are useful in the fields of industrial and consumer products and medicines, e.g., in food, human and animal drugs, and cosmetics, and the like. In some embodiments, the disclosure provides for compositions and methods for use in both human and veterinary medicine. In certain embodiments, the present disclosure relates to therapeutic treatment of mammals, birds, and fish. For example, the compositions and methods of the present disclosure are useful for therapeutic treatment of mammalian species including, but not limited to, human, bovine, ovine, porcine, equine, canine, and feline species.

Routes of administration of the delivery system include but are not limited to oral, buccal, sublingual, pulmonary, transdermal, transmucosal, as well as subcutaneous, intraperitoneal, intravenous, and intramuscular injection. Exemplary routes of administration are oral, buccal, sublingual, pulmonary, and transmucosal.

The hyperloaded YPs of the present disclosure are administered to a patient in a therapeutically effective amount. The hyperloaded YPs can be administered alone or as part of a pharmaceutically acceptable composition. In addition, a compound or composition can be administered all at once, as for example, by a bolus injection, multiple times, such as by a series of tablets, or delivered substantially uniformly over a period of time, as for example, using a controlled release formulation. It is also noted that the dose of the compound can be varied over time. The particulate delivery system can be administered using an immediate release formulation, or using a controlled release formulation, or combinations thereof The term “controlled release” includes sustained release, delayed release, and combinations thereof, as well as release mediated by chemical (e.g., pH) and/or biological (e.g., enzyme) hydrolysis.

A pharmaceutical composition of the disclosure can be prepared, packaged, or sold in bulk, as a single unit dose, or as a plurality of single unit doses. As used herein, a “unit dose” is a discrete amount of the pharmaceutical composition comprising a predetermined amount of the active ingredient. The amount of the active ingredient is generally equal to the dosage of the active ingredient that would be administered to a patient or a convenient fraction of such a dosage such as, for example, one-half or one-third of such a dosage.

The relative amounts of the active ingredient, the pharmaceutically acceptable carrier, and any additional ingredients in a pharmaceutical composition of the disclosure will vary, depending upon the identity, size, and condition of the animal or human treated, and further depending upon the route by which the composition is to be administered. By way of example, the composition can comprise between 0.1% and 100% (w/w) active ingredient. A unit dose of a pharmaceutical composition of the disclosure will generally comprise from about 100 milligrams to about 2 grams of the active ingredient, or from about 200 milligrams to about 1.0 gram of the active ingredient.

In addition, hyperloaded YPs of the present disclosure can be administered alone, in combination with hyperloaded YPs with a different payload, or with other pharmaceutically active compounds. The other pharmaceutically active compounds can be selected to treat the same condition as the hyperloaded YPs or a different condition.

If the patient is to receive or is receiving multiple pharmaceutically active compounds, the compounds can be administered simultaneously or sequentially in any order. For example, in the case of tablets, the active compounds may be found in one tablet or in separate tablets, which can be administered at once or sequentially in any order. In addition, it should be recognized that the compositions can be different forms. For example, one or more compounds may be delivered via a tablet, while another is administered via injection or orally as a syrup.

Another aspect of the disclosure relates to a kit comprising a pharmaceutical composition of the disclosure and instructional material. Instructional material includes a publication, a recording, a diagram, or any other medium of expression which is used to communicate the usefulness of the pharmaceutical composition of the disclosure for one of the purposes set forth herein in a human. The instructional material can also, for example, describe an appropriate dose of the pharmaceutical composition of the disclosure. The instructional material of the kit of the disclosure can, for example, be affixed to a container which contains a pharmaceutical composition of the disclosure or be shipped together with a container which contains the pharmaceutical composition. Alternatively, the instructional material can be shipped separately from the container with the intention that the instructional material and the pharmaceutical composition be used cooperatively by the recipient.

The disclosure also includes a kit comprising a pharmaceutical composition of the disclosure and a delivery device for delivering the composition to a human. By way of example, the delivery device can be a squeezable spray bottle, a metered-dose spray bottle, an aerosol spray device, an atomizer, a dry powder delivery device, a self-propelling solvent/powder-dispensing device, a syringe, a needle, a tampon, or a dosage-measuring container. The kit can further comprise an instructional material as described herein.

For example, a kit may comprise two separate pharmaceutical compositions comprising respectively a first composition comprising a particulate delivery system and a pharmaceutically acceptable carrier; and composition comprising second pharmaceutically active compound and a pharmaceutically acceptable carrier. The kit also comprises a container for the separate compositions, such as a divided bottle or a divided foil packet. Additional examples of containers include, without limitation, syringes, boxes, and bags. Typically, a kit comprises directions for the administration of the separate components. The kit form is advantageous when the separate components are administered in different dosage forms (e.g., oral and parenteral), are administered at different dosage intervals, or when titration of the individual components of the combination is desired by the prescribing physician.

An example of a kit is a blister pack. Blister packs are well known in the packaging industry and are being widely used for the packaging of pharmaceutical unit dosage forms, e.g., tablets and capsules. Blister packs generally consist of a sheet of relatively stiff material covered with a foil of, e.g., a transparent plastic material. During the packaging process, recesses are formed in the plastic foil. The recesses have the size and shape of the tablets or capsules to be packed. Next, the tablets or capsules are placed in the recesses and a sheet of relatively stiff material is sealed against the plastic foil at the face of the foil which is opposite from the direction in which the recesses were formed. As a result, the tablets or capsules are sealed in the recesses between the plastic foil and the sheet. The strength of the sheet is such that the tablets or capsules can be removed from the blister pack by manually applying pressure on the recesses whereby an opening is formed in the sheet at the place of the recess. The tablet or capsule can then be removed via said opening.

It may be desirable to provide a memory aid on the kit, e.g., in the form of numbers next to the tablets or capsules whereby the numbers correspond with the days of the regimen that the tablets or capsules so specified should be ingested. Another example of such a memory aid is a calendar printed on the card, e.g., as follows “First Week, Monday, Tuesday, . . . etc. . . . Second Week, Monday, Tuesday,” etc. Other variations of memory aids will be readily apparent.

Dosing can be hourly, e.g., every hour, every two hours, every four hours, every eight hours etc. Dosing can be weekly, biweekly, every four weeks, etc. A “daily dose” can be a single tablet or capsule or several pills or capsules to be taken on a given day. Also, a daily dose of a particulate delivery system composition can consist of one tablet or capsule, while a daily dose of the second compound can consist of several tablets or capsules and vice versa. The memory aid should reflect this and assist in correct administration.

In another embodiment of the present disclosure, a dispenser designed to dispense the daily doses one at a time in the order of their intended use is provided. The dispenser may be equipped with a memory aid, so as to further facilitate compliance with the dosage regimen. An example of such a memory aid is a mechanical counter, which indicates the number of daily doses that have been dispensed. Another example of such a memory aid is a battery-powered micro-chip memory coupled with a liquid crystal readout, or audible reminder signal which, for example, reads out the date that the last daily dose has been taken and/or reminds one when the next dose is to be taken.

A hyperloaded YPs composition, optionally comprising other pharmaceutically active compounds, can be administered to a patient either orally, rectally, parenterally, (for example, intravenously, intramuscularly, or subcutaneously) intracisternally, intravaginally, intraperitoneally, intravesically, locally (for example, powders, ointments or drops), or as a buccal or nasal spray.

Parenteral administration of a pharmaceutical composition includes any route of administration characterized by physical breaching of a tissue of a human and administration of the pharmaceutical composition through the breach in the tissue. Parenteral administration thus includes administration of a pharmaceutical composition by injection of the composition, by application of the composition through a surgical incision, by application of the composition through a tissue-penetrating non-surgical wound. Parenteral administration includes subcutaneous, intraperitoneal, intravenous, intraarterial, intramuscular, or intrasternal injection and intravenous, intraarterial, or kidney dialytic infusion techniques.

Compositions suitable for parenteral injection comprise the active ingredient combined with a pharmaceutically acceptable carrier such as physiologically acceptable sterile aqueous or nonaqueous solutions, dispersions, suspensions, or emulsions, or may comprise sterile powders for reconstitution into sterile injectable solutions or dispersions. Examples of suitable aqueous and nonaqueous carriers, diluents, solvents, or vehicles include water, isotonic saline, ethanol, polyols, e.g., propylene glycol, polyethylene glycol, and glycerol, and suitable mixtures thereof, triglycerides, including vegetable oils such as olive oil, or injectable organic esters such as ethyl oleate. Proper fluidity can be maintained, for example, by the use of a coating such as lecithin, by the maintenance of the required particle size in the case of dispersions, and/or by the use of surfactants. Such formulations can be prepared, packaged, or sold in a form suitable for bolus administration or for continuous administration. Injectable formulations can be prepared, packaged, or sold in unit dosage form, such as in ampules, in multi-dose containers containing a preservative, or in single-use devices for auto-injection or injection by a medical practitioner.

Formulations for parenteral administration include suspensions, solutions, emulsions in oily or aqueous vehicles, pastes, and implantable sustained-release or biodegradable formulations. Such formulations can further comprise one or more additional ingredients including suspending, stabilizing, or dispersing agents. In one embodiment of a formulation for parenteral administration, the active ingredient is provided in dry (e.g., powder or granular) form for reconstitution with a suitable vehicle (e.g., sterile pyrogen-free water) prior to parenteral administration of the reconstituted composition. The pharmaceutical compositions can be prepared, packaged, or sold in the form of a sterile injectable aqueous or oily suspension or solution. This suspension or solution can be formulated according to the known art, and can comprise, in addition to the active ingredient, additional ingredients such as the dispersing agents, wetting agents, or suspending agents described herein. Such sterile injectable formulations can be prepared using anon-toxic parenterally-acceptable diluent or solvent, such as water or 1,3-butanediol, for example. Other acceptable diluents and solvents include Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer system. Compositions for sustained release or implantation can comprise pharmaceutically acceptable polymeric or hydrophobic materials such as an emulsion, an ion exchange resin, a sparingly soluble polymer, or a sparingly soluble salt.

These compositions may also contain adjuvants such as preserving, wetting, emulsifying, and/or dispersing agents. Prevention of microorganism contamination of the compositions can be accomplished by the addition of various antibacterial and antifungal agents, for example, parabens, chlorobutanol, phenol, and sorbic acid. It may also be desirable to include isotonic agents, for example, sugars, and sodium chloride. Prolonged absorption of injectable pharmaceutical compositions can be brought about by the use of agents capable of delaying absorption, for example, aluminum monostearate and/or gelatin.

Dosage forms can include solid or injectable implants or depots. In certain embodiments, the implant comprises an aliquot of the particulate delivery system and a biodegradable polymer. In certain embodiments, a suitable biodegradable polymer can be selected from the group consisting of a polyaspartate, polyglutamate, poly(L-lactide), a poly(D,L-lactide), a poly(lactide-co-glycolide), a poly(ε-caprolactone), a polyanhydride, a poly(beta-hydroxy butyrate), a poly(ortho ester), and a polyphosphazene.

Solid dosage forms for oral administration include capsules, tablets, powders, and granules. In such solid dosage forms, the particulate delivery system is optionally admixed with at least one inert customary excipient (or carrier) such as sodium citrate or dicalcium phosphate or (a) fillers or extenders, as for example, starches, lactose, sucrose, mannitol, or silicic acid; (b) binders, as for example, carboxymethylcellulose, alginates, gelatin, polyvinylpyrrolidone, sucrose, or acacia; (c) humectants, as for example, glycerol; (d) disintegrating agents, as for example, agar-agar, calcium carbonate, potato or tapioca starch, alginic acid, certain complex silicates, or sodium carbonate; (e) solution retarders, as for example, paraffin; (f) absorption accelerators, as for example, quaternary ammonium compounds; (g) wetting agents, as for example, cetyl alcohol or glycerol monostearate; (h) adsorbents, as for example, kaolin or bentonite; and/or (i) lubricants, as for example, talc, calcium stearate, magnesium stearate, solid polyethylene glycols, sodium lauryl sulfate, or mixtures thereof. In the case of capsules and tablets, the dosage forms may also comprise buffering agents.

A tablet comprising the particulate delivery system can, for example, be made by compressing or molding the active ingredient, optionally with one or more additional ingredients. Compressed tablets can be prepared by compressing, in a suitable device, the active ingredient in a free-flowing form such as a powder or granular preparation, optionally mixed with one or more of a binder, a lubricant, an excipient, a surface-active agent, and a dispersing agent. Molded tablets can be made by molding, in a suitable device, a mixture of the active ingredient, a pharmaceutically acceptable carrier, and at least sufficient liquid to moisten the mixture. Pharmaceutically acceptable excipients used in the manufacture of tablets include inert diluents, granulating and disintegrating agents, binding agents, and lubricating agents. Known dispersing agents include potato starch and sodium starch glycolate. Known surface active agents include sodium lauryl sulfate. Known diluents include calcium carbonate, sodium carbonate, lactose, microcrystalline cellulose, calcium phosphate, calcium hydrogen phosphate, and sodium phosphate. Known granulating and disintegrating agents include corn starch and alginic acid. Known binding agents include gelatin, acacia, pre-gelatinized maize starch, polyvinylpyrrolidone, and hydroxypropyl methylcellulose. Known lubricating agents include magnesium stearate, stearic acid, silica, and talc.

Tablets can be non-coated or they can be coated using known methods to achieve delayed disintegration in the gastrointestinal tract of a human, thereby providing sustained release and absorption of the particulate delivery system, e.g. in the region of the Peyer's patches in the small intestine. By way of example, a material such as glyceryl monostearate or glyceryl distearate can be used to coat tablets. Further by way of example, tablets can be coated using methods described in U.S. Pat. Nos. 4,256,108; 4,160,452; and 4,265,874 to form osmotically-controlled release tablets. Tablets can further comprise a sweetening agent, a flavoring agent, a coloring agent, a preservative, or some combination of these in order to provide pharmaceutically elegant and palatable preparation.

Solid dosage forms such as tablets, dragees, capsules, and granules can be prepared with coatings or shells, such as enteric coatings and others well known in the art. They may also contain opacifying agents, and can also be of such composition that they release the particulate delivery system in a delayed manner. Examples of embedding compositions that can be used are polymeric substances and waxes. The active compounds can also be in micro-encapsulated form, if appropriate, with one or more of the above-mentioned excipients.

Solid compositions of a similar type may also be used as fillers in soft or hard filled gelatin capsules using such excipients as lactose or milk sugar, as well as high molecular weight polyethylene glycols. Hard capsules comprising the particulate delivery system can be made using a physiologically degradable composition, such as gelatin. Such hard capsules comprise the particulate delivery system, and can further comprise additional ingredients including, for example, an inert solid diluent such as calcium carbonate, calcium phosphate, or kaolin. Soft gelatin capsules comprising the particulate delivery system can be made using a physiologically degradable composition, such as gelatin. Such soft capsules comprise the particulate delivery system, which can be mixed with water or an oil medium such as peanut oil, liquid paraffin, or olive oil.

Oral compositions can be made, using known technology, which specifically release orally-administered agents in the small or large intestines of a human patient. For example, formulations for delivery to the gastrointestinal system, including the colon, include enteric coated systems, based, e.g., on methacrylate copolymers such as poly(methacrylic acid, methyl methacrylate), which are only soluble at pH 6 and above, so that the polymer only begins to dissolve on entry into the small intestine. The site where such polymer formulations disintegrate is dependent on the rate of intestinal transit and the amount of polymer present. For example, a relatively thick polymer coating is used for delivery to the proximal colon (Hardy et al., 1987 Aliment. Pharmacol. Therap. 1:273-280). Polymers capable of providing site-specific colonic delivery can also be used, wherein the polymer relies on the bacterial flora of the large bowel to provide enzymatic degradation of the polymer coat and hence release of the drug. For example, azopolymers (U.S. Pat. No. 4,663,308), glycosides (Friend et al., 1984, J. Med. Chem. 27:261-268) and a variety of naturally available and modified polysaccharides (see PCT application PCT/GB89/00581) can be used in such formulations.

Pulsed release technology such as that described in U.S. Pat. No. 4,777,049 can also be used to administer the particulate delivery system to a specific location within the gastrointestinal tract. Such systems permit delivery at a predetermined time and can be used to deliver the particulate delivery system, optionally together with other additives that may alter the local microenvironment to promote stability and uptake, directly without relying on external conditions other than the presence of water to provide in vivo release.

Liquid dosage forms for oral administration include pharmaceutically acceptable emulsions, solutions, suspensions, syrups, and elixirs. In addition to the active compounds, the liquid dosage form may contain inert diluents commonly used in the art, such as water or other solvents, isotonic saline, solubilizing agents and emulsifiers, as for example, ethyl alcohol, isopropyl alcohol, ethyl carbonate, ethyl acetate, benzyl alcohol, benzyl benzoate, propylene glycol, 1,3-butylene glycol, dimethylformamide, oils, e.g., almond oil, arachis oil, coconut oil, cottonseed oil, groundnut oil, corn germ oil, olive oil, castor oil, sesame seed oil, MIGLYOL™, glycerol, fractionated vegetable oils, mineral oils such as liquid paraffin, tetrahydrofurfuryl alcohol, polyethylene glycols, fatty acid esters of sorbitan, or mixtures of these substances. Besides such inert diluents, the composition can also include adjuvants, such as wetting agents, emulsifying and suspending agents, demulcents, preservatives, buffers, salts, sweetening, flavoring, coloring and perfuming agents. Suspensions, in addition to the active compound, may contain suspending agents, as for example, ethoxylated isostearyl alcohols, polyoxyethylene sorbitol or sorbitan esters, microcrystalline cellulose, hydrogenated edible fats, sodium alginate, polyvinylpyrrolidone, gum tragacanth, gum acacia, agar-agar, and cellulose derivatives such as sodium carboxymethylcellulose, methylcellulose, hydroxypropylmethylcellulose, aluminum metahydroxide, bentonite, or mixtures of these substances. Liquid formulations of a pharmaceutical composition of the disclosure that are suitable for oral administration can be prepared, packaged, and sold either in liquid form or in the form of a dry product intended for reconstitution with water or another suitable vehicle prior to use.

Known dispersing or wetting agents include naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g. polyoxyethylene stearate, heptadecaethyleneoxycetanol, polyoxyethylene sorbitol monooleate, and polyoxyethylene sorbitan monooleate, respectively). Known emulsifying agents include lecithin and acacia. Known preservatives include methyl, ethyl, or n-propyl-para-hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

For topical administration liquids, suspension, lotions, creams, gels, ointments, drops, suppositories, sprays and powders may be used. Conventional pharmaceutical carriers, aqueous, powder or oily bases, and thickeners can be used as necessary or desirable.

In other embodiments, the pharmaceutical composition can be prepared as a nutraceutical, i.e., in the form of, or added to, a food (e.g., a processed item intended for direct consumption) or a foodstuff (e.g., an edible ingredient intended for incorporation into a food prior to ingestion). Examples of suitable foods include candies such as lollipops, baked goods such as crackers, breads, cookies, and snack cakes, whole, pureed, or mashed fruits and vegetables, beverages, and processed meat products. Examples of suitable foodstuffs include milled grains and sugars, spices and other seasonings, and syrups. The particulate delivery systems described herein are not exposed to high cooking temperatures for extended periods of time, in order to minimize degradation of the compounds.

Compositions for rectal or vaginal administration can be prepared by mixing a particulate delivery system with suitable non-irritating excipients or carriers such as cocoa butter, polyethylene glycol or a suppository wax, which are solid at ordinary room temperature, but liquid at body temperature, and therefore, melt in the rectum or vaginal cavity and release the particulate delivery system. Such a composition can be in the form of, for example, a suppository, a retention enema preparation, and a solution for rectal or colonic irrigation. Suppository formulations can further comprise various additional ingredients including antioxidants and preservatives. Retention enema preparations or solutions for rectal or colonic irrigation can be made by combining the active ingredient with a pharmaceutically acceptable liquid carrier. As is known in the art, enema preparations can be administered using, and can be packaged within, a delivery device adapted to the rectal anatomy of a human. Enema preparations can further comprise various additional ingredients including antioxidants and preservatives.

A pharmaceutical composition of the disclosure can be prepared, packaged, or sold in a formulation suitable for pulmonary administration via the buccal cavity. Such compositions are conveniently in the form of dry powders for administration using a device comprising a dry powder reservoir to which a stream of propellant can be directed to disperse the powder or using a self-propelling solvent/powder-dispensing container such as a device comprising the particulate delivery system suspended in a low-boiling propellant in a sealed container. Dry powder compositions may include a solid fine powder diluent such as sugar and are conveniently provided in a unit dose form. Low boiling propellants generally include liquid propellants having a boiling point below 65 degrees F at atmospheric pressure. Generally, the propellant can constitute 50 to 99.9% (w/w) of the composition, and the active ingredient can constitute 0.1 to 20% (w/w) of the composition. The propellant can further comprise additional ingredients such as a liquid non-ionic or solid anionic surfactant or a solid diluent, e.g., having a particle size of the same order as particles comprising the particulate delivery system.

Pharmaceutical compositions of the disclosure formulated for pulmonary delivery can also provide the active ingredient in the form of droplets of a suspension. Such formulations can be prepared, packaged, or sold as aqueous or dilute alcoholic suspensions, optionally sterile, comprising the particulate delivery system, and can conveniently be administered using any nebulization or atomization device. Such formulations can further comprise one or more additional ingredients including a flavoring agent such as saccharin sodium, a volatile oil, a buffering agent, a surface-active agent, or a preservative such as methylhydroxybenzoate.

The formulations described herein as being useful for pulmonary delivery are also useful for intranasal delivery of a pharmaceutical composition of the disclosure. Another formulation suitable for intranasal administration is a coarse powder comprising the particulate delivery system. Such a formulation is administered in the manner in which snuff is taken, i.e., by rapid inhalation through the nasal passage from a container of the powder held close to the nares.

A pharmaceutical composition of the disclosure can be prepared, packaged, or sold in a formulation suitable for buccal administration. Such formulations can, for example, be in the form of tablets or lozenges made using conventional methods, and can, for example, comprise 0.1 to 20% (w/w) particulate delivery system, the balance comprising an orally dissolvable or degradable composition and, optionally, one or more of the additional ingredients described herein. Alternately, formulations suitable for buccal administration can comprise a powder or an aerosolized or atomized solution or suspension comprising the particulate delivery system.

Reference will now be made to specific examples illustrating the disclosure. It is to be understood that the examples are provided to illustrate exemplary embodiments and that no limitation to the scope of the disclosure is intended thereby.

### EXAMPLES

### Example 1: Production of Yeast Particles

YPs are typically 3-5 μm hollow and porous microparticles derived from Baker's yeast that are composed primarily of ˜80% 1→6β branched, 1→6β-glucan, 2-4% chitin and 40% mannan w/w. Yeast particles are readily available, biodegradable, substantially spherical particles about 2-4 μm in diameter.

Methods of preparing extracted yeast cell wall particles are known in the art, and are described, for example, in U.S. Pat. Nos. 4,992,540, 5,082,936, 5,028,703, 5,032,401, 5,401,727, 5,504,079, 5,968,811, 6,444,448 B1, 6,476,003 B1, published U.S. applications 2003/0216346 A1, 2004/0014715 A1, and published PCT application WO 02/12348 A2, the disclosures of which are incorporated herein by reference.

A form of extracted yeast cell wall particles, referred to as “whole glucan particles” or “WPGs” (See U.S. Pat. Nos. 5,032,401 and 5,607,677) may be modified to facilitate improved retention and/or delivery of payload molecules. Such improvements feature trapping molecules and nanoparticles as well as pluralities of said trapping molecules and nanoparticles, formulated in specific forms to achieve the desired improved delivery properties. As used herein, a WGP is typically a whole glucan particle of >90% beta glucan purity.

### Preparation of Glucan Particles (GPs)

Glucan particles (GPs), also referred to herein as yeast glucan particles (“YGPs”), are a purified hollow yeast cell ‘ghost’ containing a rich β-glucan sphere, generally 2-4 microns in diameter. In general, glucan particles can be prepared from yeast cells by the extraction and purification of the alkali-insoluble glucan fraction from the yeast cell walls. The yeast cells can be treated with an aqueous hydroxide solution without disrupting the yeast cell walls, which digests the protein and intracellular portion of the cell, leaving the glucan wall component devoid of significant protein contamination, and having substantially the unaltered cell wall structure of β(1-6) and β(1-3) linked glucans. The 1,3-β-glucan outer shell provides for receptor-mediated uptake by phagocytic cells, e.g., macrophages, expressing β-glucan receptors.

Glucan particles can be made as follows. Yeast particles (S. cerevisiae), Biorigin MOS55, are suspended in 1 liter of 1M NaOH and heated to 85° C. The cell suspension is stirred vigorously for 1 hour at this temperature. The insoluble material containing the cell walls is recovered by centrifuging. This material is then suspended in 1M NaOH, heated, and stirred vigorously for 1 hour. The suspension is allowed to cool to room temperature and the extraction is continued for a further 16 hours. The insoluble residue is recovered by centrifugation. This material is finally extracted in water brought to pH 4.5 with HC1. The insoluble residue is recovered by centrifugation and washed three times with water, isopropanol, and acetone. The resulting slurry is placed in glass trays and dried under reduced pressure to produce a fine white powder.

### Preparation of Glucan Lipid Particles (GLPs)

GLPs retain some of the yeast cellular lipid content, which creates a more hydrophobic inner cavity ideal for loading of hydrophobic payloads. GLPs are prepared by modifying the method of preparation of GPs described above. For preparation of GLPs, washing with isopropanol and acetone is eliminated and instead the insoluble residue recovered by centrifugation is washed three times with water. The particles are dried by lyophilization or spray drying.

### Commercial Yeast Particles (YPs)

Yeast particles (YPs) were purchased from Biorigin (Louisville, KY, USA) or LeSaffre (Marcq-en-Barceul, France). These YPs contained sufficient amounts of lipids to provide for a hydrophobic reservoir that attracts hydrophobic payloads to diffuse into the center of the particle accomplishing loading.

### Whole Glucan Particles (WGPs)

A more detailed description of processes for preparing WPGs can be found in U.S. Patent Nos. 4,810,646, 4,992,540, 5,028,703, 5,607,677, and 5,741,495 (incorporated herein by reference). For example, U.S. Pat. No. 5,028,703 discloses that yeast WGP particles can be produced from yeast strain R4 cells in fermentation culture. The cells are harvested by batch centrifugation at 8000 rpm for 20 minutes in a Sorval RC2-B centrifuge. The cells are washed twice in distilled water in order to prepare them for the extraction of the whole glucan. The first step involved resuspending the cell mass in 1 liter 4% w/v NaOH and heating to 100° C. The cell suspension is stirred vigorously for 1 hour at this temperature. The insoluble material containing the cell walls is recovered by centrifuging at 2000 rpm for 15 minutes. This material is suspended in 2 liters, 3% w/v NaOH and heated to 75° C. The suspension is stirred vigorously for 3 hours at this temperature. The suspension id then allowed to cool to room temperature and the extraction can be continued for a further 16 hours. The insoluble residue is recovered by centrifugation at 2000 rpm for 15 minutes. This material is finally extracted in 2 liters, 3% w/v NaOH brought to pH 4.5 with HCl, at 75° C. for 1 hour. The insoluble residue is recovered by centrifugation and washed three times with 200 milliliters water, once with 200 milliliters dehydrated ethanol, and twice with 200 milliliters dehydrated ethyl ether. The resulting slurry is placed on petri plates and dried.

Varying degrees of purity of glucan particles are achieved by modifying the extraction/purification process. In general, these GPs are on the order of 80-85% pure on a w/w basis of beta glucan and, following the introduction of payload, trapping, or other components, become of a slightly lesser “purity.” In exemplary embodiments, GPs are <90% beta glucan purity.

### Preparation of YCP Particles

Yeast cells (Rhodotorula sp.) derived from cultures obtained from the American Type Culture Collection (ATCC, Manassas, Va.) are aerobically grown to stationary phase in YPD at 30° C. Rhodotorula sp. cultures available from ATCC include Nos. 886, 917, 9336, 18101, 20254, 20837 and 28983. Cells are harvested by batch centrifugation at 2000 rpm for 10 minutes. The cells are then washed once in distilled water and then re-suspended in water brought to pH 4.5 with HCl, at 75° C. for 1 hour. The insoluble material containing the cell walls is recovered by centrifuging. This material is then suspended in 1 liter, 1M NaOH and heated to 90° C. for 1 hour. The suspension is allowed to cool to room temperature and the extraction is continued for a further 16 hours. The insoluble residue is recovered by centrifugation and washed twice with water, isopropanol, and acetone. The resulting slurry is placed in glass trays and dried at room temperature to produce 2.7 g of a fine light brown powder.

In alternative embodiments, YGPs, e.g., activated YGPs, are grafted with chitosan on the surface, for example, to increase total surface chitosan. Chitosan can further be acetylated to form chitin (YGCP), in certain embodiments. Such particles have equivalent properties in vivo when detected by the immune system of a subject or patient.

### Preparation of YGMP Particles

S. cerevisiae (100 g Fleishman's Baker's yeast) was suspended in 1 liter 1M NaOH and heated to 55° C. The cell suspension was mixed for 1 hour at this temperature. The insoluble material containing the cell walls was recovered by centrifuging at 2000 rpm for 10 minutes. This material was then suspended in 1 liter of water and brought to pH 4-5 with HCl, and incubated at 55° C. for 1 hour. The insoluble residue was recovered by centrifugation and washed once with 1000 milliliters water, four times with 200 milliliters dehydrated isopropanol and twice with 200 milliliters acetone. The resulting slurry was placed in a glass tray and dried at room temperature to produce 12.4 g of a fine, slightly off-white, powder.

S. cerevisiae (75 g SAF-Mannan) was suspended in 1 liter water and adjusted to pH 12-12.5 with 1M NaOH and heated to 55° C. The cell suspension was mixed for 1 hour at this temperature. The insoluble material containing the cell walls was recovered by centrifuging at 2000 rpm for 10 minutes. This material was then suspended in 1 liter of water and brought to pH 4-5 with HCl, and incubated at 55° C. for 1 hour. The insoluble residue was recovered by centrifugation and washed once with water, dehydrated isopropanol, and acetone. The resulting slurry was placed in a glass tray and dried at room temperature to produce 15.6 g of a fine slightly off-white powder.

### Measurement of Yeast Particle Diameter

Microscopy images of YP control and YP-GET samples were obtained at 1,000× magnification. An image of a microscope calibration slide ruler was used to set the scale in pixels/pm in ImageJ software. The photomicrographs of YP samples were evaluated with the calibrated scale in ImageJ. The particle diameter along the major and minor axes of the YP ellipses was measured for 20 whole yeast cell particles per picture and a minimum of three pictures per sample.

### Example 2: Loading of Terpene Payload into Yeast Particle without a Solvent

Payloads that are water insoluble or low water-soluble payloads with a melting point <70° C. can be loaded in YP without using an organic solvent. This loading method is achieved in a single step and yields maximizes loading capacity of YP up to 5:1 payload:YP weight ratio. Table 1 shows examples of payloads that can loaded in YPs without a solvent.

TABLE 1
Payloads that be loaded in YPs without a solvent.

| | | | | Maximum | |
|---|---|---|---|---|---|
| | | Melting | Density | solubility | |
| Compound | Log P | point (° C.) | (g/mL) | in water | |
| Eugenol | 2.3 | −9.1 | 1.06 | 1 | mg/mL |
| Clomazone | 2.5 | 25 | 1.192 | 1.1 | mg/mL |
| Thymol | 3.3 | 51.5 | 0.96 | 0.9 | mg/mL |
| Geraniol | 3.6 | −15 | 0.889 | 0.686 | mg/mL |
| Triallate | 4.6 | 34 | 1.27 | 4 | μg/mL |
| Limonene | 4.6 | −74 | 0.841 | 7.57 | μg/mL |
| Tetrahydrocannabinol | 5.9 | <25 | — | 2.8 | μg/mL |
| (THC) | | | | | |
| Lambda cyhalotrin | 7.0 | 49.2 | 1.3 | 5 | ng/ml |

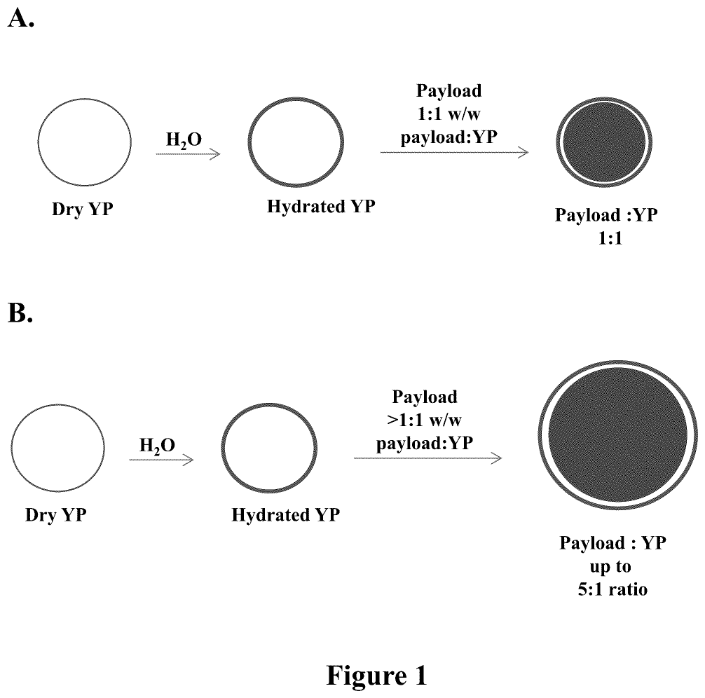

FIG. 1 depicts the difference between standard loading and hyperloading of YP. Addition of a minimum amount of water (0.5 μL water per mg YP) was required to swell dry YPs. Swelling of the YPs facilitates passive diffusion of payloads into the interior of the YPs through the pores in the YP shell. Incubation of payload at 1:1 weight ratio with YP results in loading of payload to yield loaded particles with 1:1 ratio of YP:payload (FIG. 1A). Incubation of hydrated YP with increased amounts of payload, i.e., payload:YP weight/weight ratio of greater than 1:1, promotes continual diffusion of hydrophobic payload inside the YP which further swells the YPs allowing for hyperloading of YPs to a weight/weight payload:YP ratio of 5:1 (FIG. 1B). Kinetics of the payload loading in YP is a function of YP lipid content, amount of water used to swell the particles, temperature and solubility of the payload.
Loading Terpenes into YPs

The porous cell wall structure makes these particles excellent absorbent materials, and hydrophobic payloads could be loaded from aqueous and some organic solutions with high payload loading capacity into the large hollow YP cavity. For example, terpenes could be encapsulated in the hydrophobic cavity of YPs by the passive diffusion of the payload through the porous cell walls as depicted in FIG. 1.

A mixture of three terpenes (geraniol, eugenol and thymol or GET) at a composition of 2:1:2 G:E:T weight ratio was used. This GET composition was highly effective in antifungal and antinematicidal agricultural applications against a broad range of plant pathogens. The chemical structures, water/octanol partition coefficient (log P) and solubility in water of the selected terpenes are shown in Table 2.

TABLE 2
Chemical structure, water/octanol partition coefficient (log P) and solubility
in water of the terpenes used for loading in YPs.

| | | Solubility in |
|---|---|---|
| | | water |
| Terpene | Log P | (mg/mL) |
| 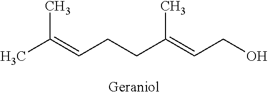 | 3.56 | 0.686 |
| 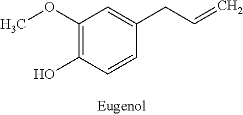 | 2.27 | 1.44 |
| 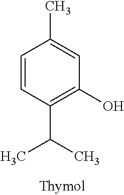 | 3.3 | 0.9 |

YP Loading of Terpenes (Terpene:YP w/w ratio of 1.1:1) Dry YPs were mixed with water (180 g YP/L) and the slurry was passed through an EMULSIFLEX®-C3 high pressure homogenizer (Avestin, Ottawa, ON) to obtain a uniform, single YP suspension. Samples of homogenized YP (8.35 g) were mixed with a geraniol-eugenol-thymol (GET) mixture (1.65 g GET at a composition of 2:1:2 GET weight ratio) and incubated at room temperature for a minimum of 24 h to allow for complete terpene loading by diffusion through the porous yeast cell wall into the hydrophobic hollow interior.

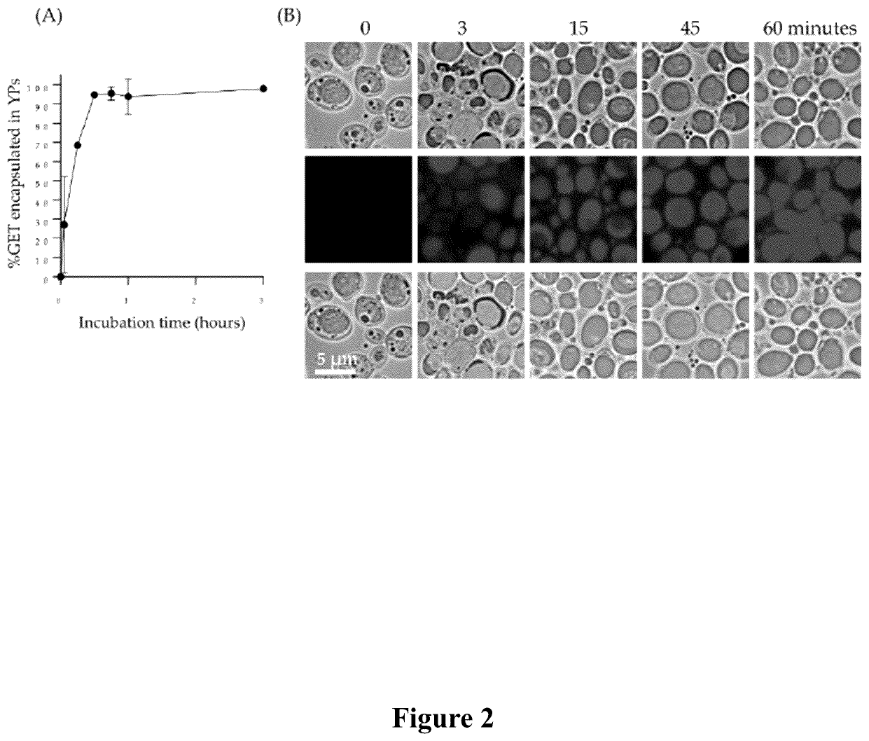

Terpene encapsulation in YPs is achieved with high encapsulation efficiency and homogenous terpene distribution in the particles. The passive diffusion of the GET mixture into YPs in a homogenized aqueous YP suspension (GET:YP weight target ratio of 1.1:1) was a rapid process and >95% of the terpenes were encapsulated in YPs within one hour as shown by HPLC quantification and microscopy in FIG. 2.

### YP Loading of Terpenes (Terpene:YP w/w Ratio≥2:1)

Dry YPs were mixed with water for 30 minutes to obtain a uniform hydrated YP suspension (terpene loading at high terpene:YP ratios does not require homogenization of YPs) and then a 2:1:2 GET mixture was added to the YP sample and incubated at room temperature for a minimum of 24 h to allow for complete terpene loading. The amounts of YP, water and terpene required to prepare YP GET with weight ratios of 1.1:1 to 5:1 are indicated in Table 3.

TABLE 3
Yeast particle, water and 2:1:2 GET quantities
required to produce 100 g of YP GET at five
GET:YP ratios at a constant YP concentration.

| GET:YP | Materials to produce 100 g YP-GET | | | Final composition | |
|---|---|---|---|---|---|
| ratio | g YP | mL (g) water | g GET | % YP | % GET |
| 1.1:1 | 15 | 68.5 | 16.5 | 15 | 16.5 |
| 2:1 | 15 | 55 | 30 | 15 | 30 |
| 3:1 | 15 | 40 | 45 | 15 | 45 |
| 4:1 | 15 | 25 | 60 | 15 | 60 |
| 5:1 | 15 | 10 | 75 | 15 | 75 |

### Characterization of Terpene Loading Efficiency

Samples of YP-GET (10 μL, 10 mg YP/mL) were stained with Nile red (2 μL, 0.1 mg/mL) and fluorescein labeled concanavalin-A (FITC ConA, 2 μL, 0.1 mg/mL) to qualitatively assess loading by the fluorescence microscopy of the encapsulated fluorescent terpene-Nile red complex in the FITC ConA labeled yeast particle. Nile red was imaged using a rhodamine (red) filter (maxi-mum excitation/emission wavelengths at 550/570 nm) and FITC-ConA was imaged with a green filter at 490/520 nm. Microscopy images were collected with an Olympus BX60 upright compound fluorescent microscope. YP GET samples (100 mg) were centrifuged to collect excess liquid (free terpene and water) and the pellet fraction was resuspended in 10 mL of 90% methanol-10% water to extract encapsulated terpenes. Terpenes were quantified by HPLC operated with 32 KARAT™ software version 7.0 (Beckman Coulter, Inc, Brea, CA, USA), using a Waters SYMMETRY® C18 column (3.5 μm, 4.6×150 mm) with acetonitrile:water 50:50 as mobile phase, flow rate at 1 mL/min, injection volume of 10 μL, and terpene detection at 254 nm. This isocratic HPLC method allowed for the detection of the three terpenes in the GET mixture in a single run with the following retention times: 5.2 minutes (eugenol), 7.7 minutes (geraniol), and 9.8 minutes (thymol). The quantification of terpenes was done by measuring the peak area and interpolating the concentration using a calibration curve obtained with terpene standards.

Terpenes were mixed with water and YPs from two different manufacturers and incubated at room temperature for 48 hours and encapsulation efficiency of terpenes in YPs was measured as described above after incubation at room temperature for 2 days. Table 4 shows the terpene (GET212) encapsulation efficiency in commercially acquired YPs. Results showed that GET212 could be efficiently loaded in YP up to a GET212:YP ratio of 5:1.

TABLE 4
Terpene (GET212) encapsulation efficiency in YPs

| | | | Incubation | |
|---|---|---|---|---|
| GET424*:YP | | mL H₂O/g | time at | % GET424 |
| ratio | YP | YP | 23° C. | in YP |
| 1.1:1 | Biorigin | 4.5 | 2 days | 99.9 |
| 2:1 | Biorigin | 3.5 | 2 days | 100 |
| 3:1 | Biorigin | 2.5 | 2 days | 100 |
| 4:1 | Biorigin | 1.5 | 2 days | 99 |
| 5:1 | Biorigin | 0.5 | 2 days | 97 |
| 1.1:1 | SAF Mannan | 4.5 | 2 days | 99.9 |
| 2:1 | SAF Mannan | 3.5 | 2 days | 100 |
| 3:1 | SAF Mannan | 2.5 | 2 days | 100 |
| 4:1 | SAF Mannan | 1.5 | 2 days | 99 |
| 5:1 | SAF Mannan | 0.5 | 2 days | 98 |

*GET424: a mixture of geraniol (G), eugenol (E), thymol (T) at a weight ratio composition of 2:1:2

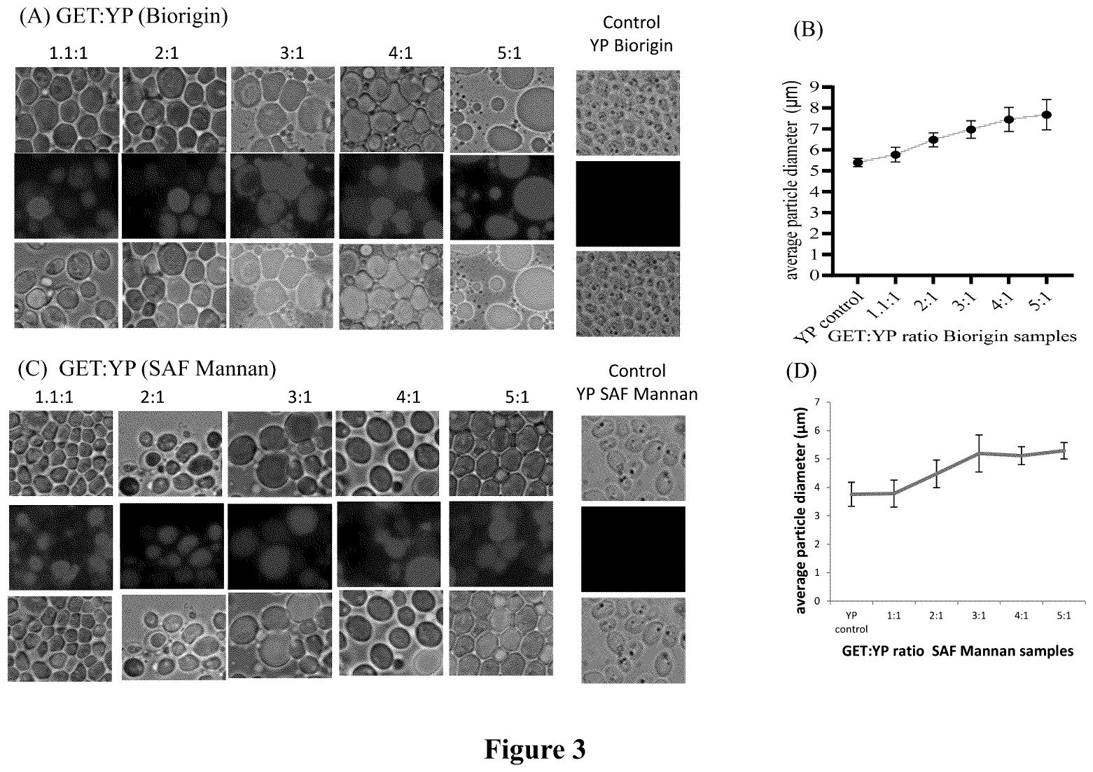

Hyperloading of YPs prepared using increasing ratios of GET:YP led to swelling of YPs as seen in light and fluorescent micrographs (FIGS. 3A, 3C). Swelling of YPs was corroborated for particle size measurements. The average particle diameter (diameter of major axis of the YP ellipsoids) increased from 5.4 μm (empty YPs) to 7.7 μm (YP GET 5:1) as shown in FIG. 3B and 3D.

### Example 3: Release of Terpenes from Hyperloaded YP

Sustained payload release from YPs occurs by payload diffusion out of the particles and is a function of the payload's solubility in water.

Terpene Release from YP-GET

YP-GET samples (100 mg) were suspended in water (10 mL) and incubated at room temperature for 24 h. Aliquots were collected at predetermined times, centrifuged and the supernatant collected to measure terpene released from the particles by HPLC. The initial YP-GET suspension in water (1.5 mg YP/mL) was diluted two-fold, incubated at room temperature for another 24 h and samples collected to quantify terpene release from YPs. Additional two-fold dilutions were done every 24 h until achieving >90% release from YPs.

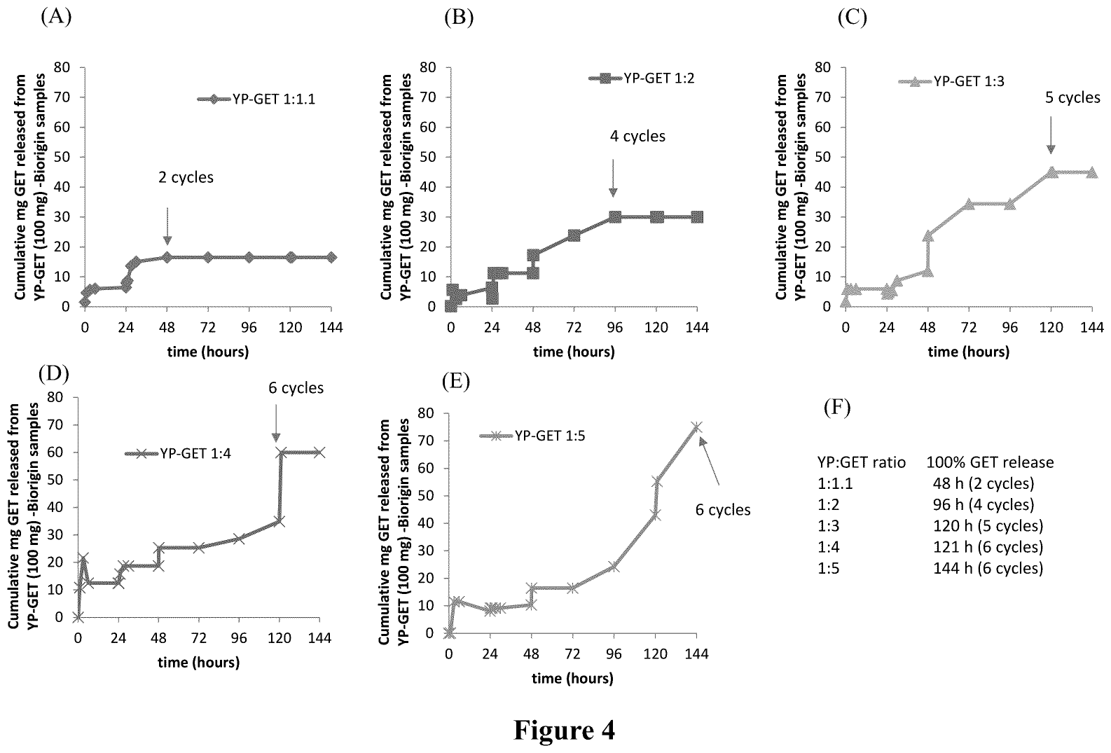

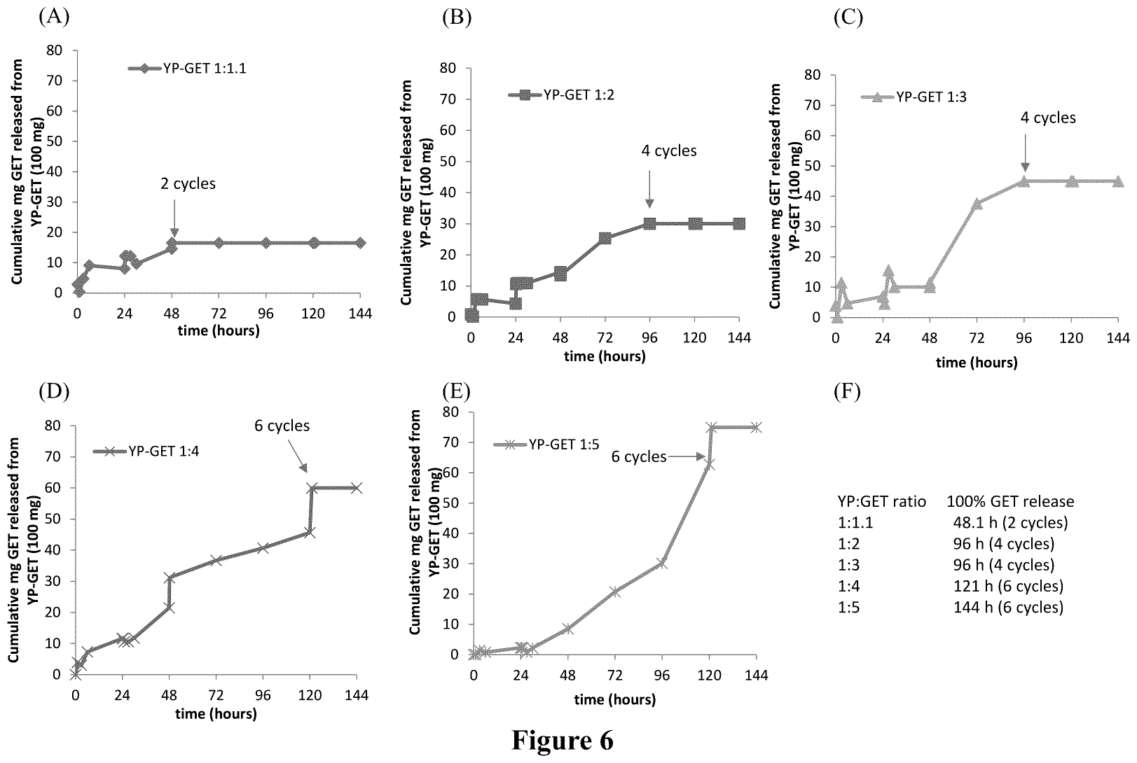

The terpenes in the GET composition had maximum solubility in water of 0.686 (geraniol), 0.9 (thymol) and 1.44 mg/mL (eugenol). A 1:100 dilution of a YP-GET 1:1.1 (150 mg YP/mL, 165 mg GET/mL) generated a sample with a total GET concentration in water of 1.65 mg GET/mL. The concentration of each terpene was below its maximum solubility in water at this 1:100 dilution. The results in FIGS. 4A and 6A for YP-GET 1.1:1 show sustained release of ˜7.5 mg (<50% GET content in a 100 mg YP-GET 1.1:1 sample) during the first 6 h incubation and no additional release from 6 to 24 h. Without intending to be bound by scientific theory, the plateau after six hours indicated that some GET was retained in the particles, likely due to interactions with hydrophobic lipids in the YP cell walls. A second dilution (1:1) was done at 24 h to achieve complete GET release from YP-GET 1.1:1.

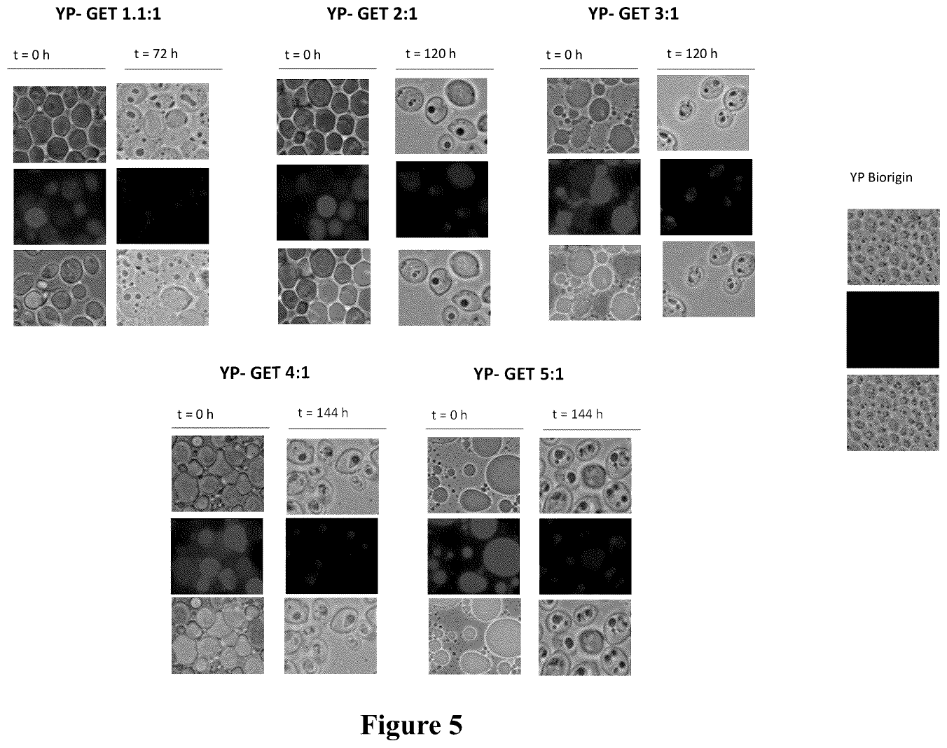

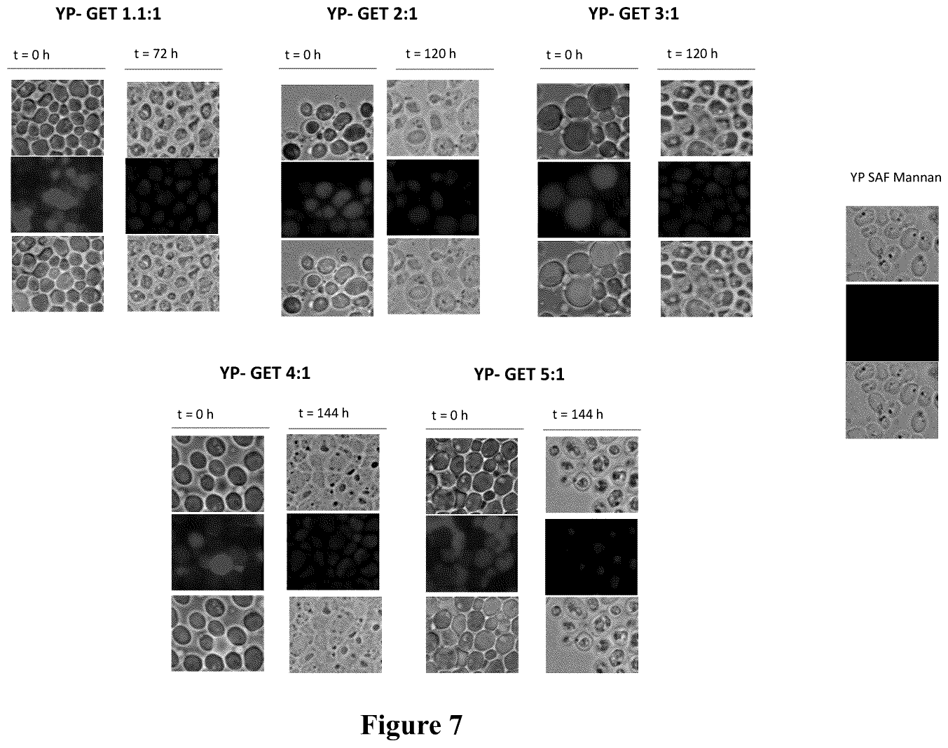

The hyperloaded YP-GET samples were evaluated with the same procedure starting with a constant amount of YP-GET (100 mg) diluted in 10 mL to generate samples of varying GET content from 16.5 mg up to 75 mg GET. The samples were diluted 1:1 every 24 hours to determine the number of cycles to achieve complete GET release from YPs (FIG. 4B-4E, FIG. 6B-6E). The GET release results show: (1) hyperloaded YP-GET were stable, as no burst leading to release of an emulsion of terpenes in water and empty YPs was observed upon dilution; and (2) it was possible to extend the number of wetting/terpene release cycles three-fold from the two cycles for the commercialized YP GET 1.1:1 up to six cycles for the hyperloaded YP GET 4:1 and 5:1 formulations (FIGS. 4F and 5F). Light and fluorescent photomicrographs in FIG. 5 (YPs from Biorigin) and FIG. 7 (YPs from SAF Mannan) show that GET was completely released from the hyperloaded YPs upon repeated cycles of dilution.

The analysis of the GET released from the particles showed a similar release pattern for each terpene in each of the five YP GET samples, indicating that the 3 terpenes were releasing together as an isotropic mixture and not differentially releasing based on their water solubility, as eugenol and thymol are significantly more water soluble than geraniol. This is important for the consistent antimicrobial bioactivity of this mixture of terpenes released over time.

The kinetics of terpene release from YPs was also evaluated in water to simulate release conditions in rainwater and ambient humidity and in 0.9% saline to simulate groundwater and biological fluids. The salt concentration had no effect on terpene release.

Such sustained release of terpenes could significantly enhance efficacy and time between spraying for agricultural applications of YP-GET in soils, field crops, post-harvest decay and seeds treatments.

### Example 4: Testing Stability of Encapsulated Payload

### YP-GET Stability to Shearing by Sonication

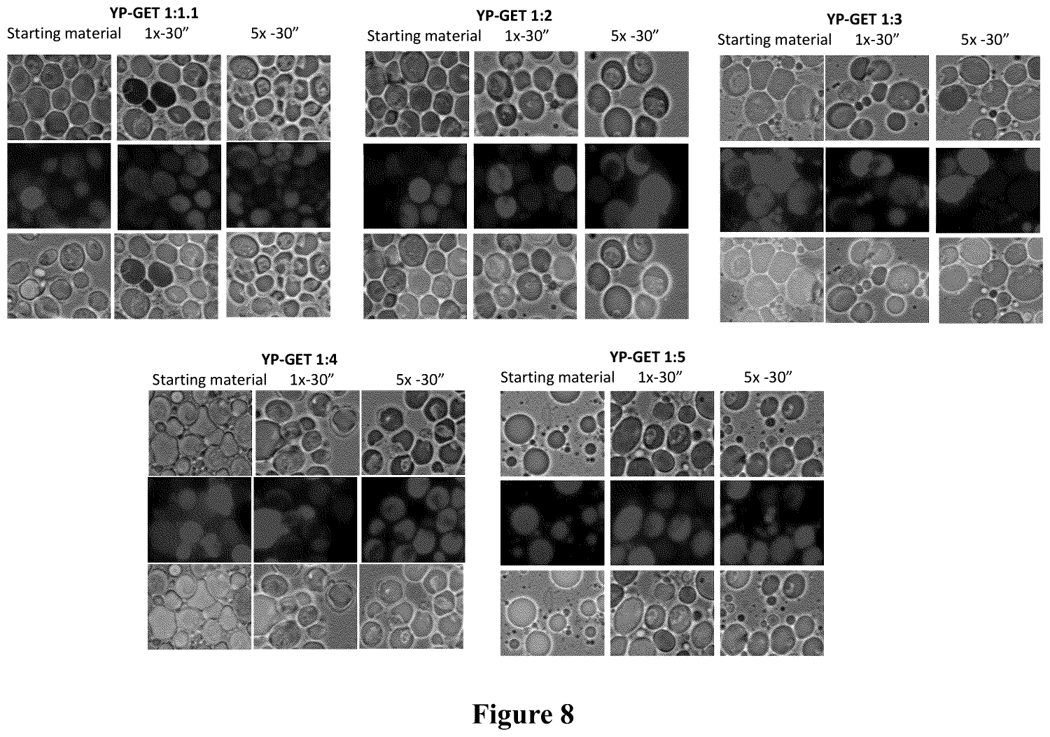

YP-GET424 (a mixture of geraniol (G), eugenol (E), thymol (T) at a weight ratio composition of 2:1:2) samples were sonicated one or five times for 30 seconds. Samples were centrifuged and free GET in the supernatant was measured by HPLC. Table 5 shows that all YP-GET424 remain well loaded and retained the payload despite shear stress introduced by sonication. Light and fluorescent photomicrographs shown in FIG. 8 confirmed that the terpene payload remained intact within the YP after sonication.

TABLE 5
Encapsulation Stability of YP-GET

| | | | % GET retained in YP | |
|---|---|---|---|---|
| | | | formulation | |
| | | | YP-GET42*4 | YP-GET424* |
| GET:YP | | % GET YP | Sonication: | Sonication: |
| ratio | YP | (t = 0) | 1X - 30 sec | 5X-30 sec |
| 1.1:1 | Biorigin | 99.9 | 91.3 | 98.3 |
| 2:1 | Biorigin | 100 | 100 | 97.1 |
| 3:1 | Biorigin | 100 | 99.7 | 100 |
| 4:1 | Biorigin | 100 | 100 | 99.2 |
| 5:1 | Biorigin | 100 | 98.1 | 97.1 |

*GET424: a mixture of geraniol (G), eugenol (E), thymol (T) at a weight ratio composition of 2:1:2

### YP-GET Encapsulation Storage Stability

YP-GET samples (500 mg) were transferred into glass vials and stored at room temperature (23° C.) or at 54° C. for two weeks. A third set of samples was subjected to three freeze (−20° C.)/thaw (23° C.) cycles. The encapsulation storage stability was assessed by Nile-red microscopy, yeast particle diameter measurement using ImageJ software, particle counting with a hemocytometer, and quantification of free and encapsulated terpene by HPLC before and after storage.

Results of HPLC quantification of free GET before and after storage are summarized in Table 6. YP-GET samples had diminished stability after free-thaw cycles and prolonged storage at 54° C.

TABLE 6
Quantification of free GET by HPLC before and after storage.

| | | | | % GET retained in YP | | |
|---|---|---|---|---|---|---|
| | | | | formulation | | |
| | | | | YP-GET | | |
| | | | % | frozen | YP-GET | YP-GET |
| | | | GET | (−20° C.) | stored 2 | heated at |
| | GET:YP | | YP | & thawed | weeks at | 54° C. for |
| g YP/L | ratio | YP | (t = 0) | 3 times | RT* | 2 weeks |
| 150 | 1.1:1 | Biorigin | 99.9 | 81 ± 2 | 99 ± 1 | 83 ± 9 |
| 150 | 2:1 | Biorigin | 100 | 87 ± 3 | 92 ± 5 | 91 ± 12 |
| 150 | 3:1 | Biorigin | 100 | 83 ± 4 | 88 ± 6 | 72 ± 6 |
| 150 | 4:1 | Biorigin | 100 | 56 ± 4 | 88 ± 5 | 64 ± 6 |
| 150 | 5:1 | Biorigin | 100 | 65 ± 18 | 81 ± 9 | 76 ± 7 |

*Room Temperature (23° C.)

### Example 5: Increasing Storage Stability of Encapsulated Payload

### Stabilization of Hyperloaded YP-GET

The major reason for YP-GET instability was the appearance of broken shells prematurely releasing their terpene contents. YP-GET could be stabilized for storage at low and high temperature by incorporation of a cryoprotectant during loading of GET in YPs. Glycerin is a GRAS compound used as cryoprotectant in biological applications, such as low temperature storage of blood cells. Glycerin works as a cryoprotectant by forming strong hydrogen bonds with water. A mixture of 30% glycerin and 70% water has a freezing point of −38.9° C. and a boiling point of 114° C. and therefore is a suitable mixture of solvents to improve temperature encapsulation stability of YP-GET. It was possible to prepare YP-GET samples using 30% glycerin at YP concentration of 100 g/L and GET:YP ratios of 3:1 up to 4.5:1.

Dry YP was mixed with water and glycerin (70% water, 30% glycerin) for 30 minutes to obtain a uniform hydrated YP suspension, and then 2:1:2 GET was added to the YP sample and incubated at room temperature for a minimum of 24 h to allow for complete terpene loading. YP-GET samples in water (control) and water-glycerin at YP concentration of 100 g YP/L and GET:YP ratios from 3:1 up to 4.5:1 were prepared. Terpene encapsulation efficiency, encapsulation storage stability, and particle size were measured as described above.

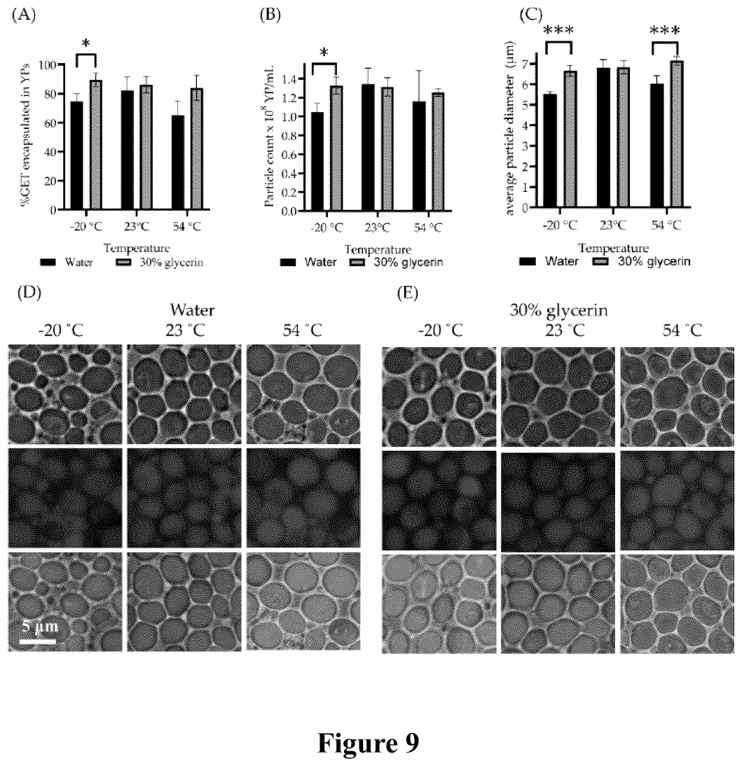

These samples were produced with high encapsulation efficiency (>90%) and the use of 30% glycerin as solvent did not impact GET release. Stabilized YP-GET samples showed enhanced storage encapsulation stability following temperature stress. The results in Table 7 and FIG. 9 show the encapsulation stability results of YP-GET samples prepared in water and in 30% glycerin-70% water. The results showed that samples prepared in 30% glycerin were more stable at all three temperatures than the YP-GET samples without glycerin. YP-GET samples prepared in water showed a reduction in particle number (FIG. 9B) after storage at −20° C. due to particle breakage by ice crystals, and also a reduction in amount of encapsulated terpene (FIG. 9A) and average YP diameter due to partial GET loss (FIG. 9C) at both −20° C. and 54° C. Light and fluorescent photomicrographs confirmed that hyperloaded YP-GET samples prepared in in 30% glycerin-70% water (FIG. 9E) were more stable during storage at different temperatures than YP-GET samples prepared in water (FIG. 9D).

TABLE 7
Temperature stabilization of YP-GET by 30% glycerol

| | | | Storage for two weeks at | | | | | |
|---|---|---|---|---|---|---|---|---|
| | | | −20° C. | | 25° C. | | 54° C. | |
| | | | | YP | | YP | | YP |
| | | | | count | | count | | count |
| g | | | % | (×10⁸ | % | (×10⁸ | 5 | (×10⁸ |
| YP/ | Loading | GET:YP | GET | YPs/ | GET | YPs/m | GET | YPs/ |
| L | solvent | ratio | in YP | mL) | in YP | L) | in YP | mL) |
| 100 | Water | 3.5:1 | 74.5 ± | 1.15 ± | 82.2 ± | 1.21 ± | 65 ± | 1.01 ± |
| | | | 5.6 | 0.03 | 9.1 | 0.06 | 10.1 | 0.14 |
| 100 | 30% | 3.5:1 | 89.5 ± | 1.27 ± | 86.2 ± | 1.23 ± | 83.9 ± | 1.21 ± |
| | glycerin | | 4.6 | 0.04 | 5.7 | 0.04 | 8.7 | 0.12 |

### Optimization of Hyperloaded YP-GET

Hyperloaded YP-GET samples prepared with a final compositions of 15% w/w YP exhibit poor flowability at ratios ≥3:1 GET:YP preventing their application as aqueous suspension concentrates. The flow rate of a YP-GET 1:3 at 15% YP is 0.0003 cm³/s, which represents an ˜80% reduction compared to the flow rate of YP-GET 1.1:1 at 15% YP. Hyperloaded YP-GET samples ≥3:1 GET:YP could be processed into dry YP-GET granules by an extrusion process or could be prepared at lower YP concentrations to improve the flow rate of the final product and applied as YP-GET aqueous suspensions concentrate. Samples with lower YP concentration were prepared to improve the flow rate of the final product. YP-GET samples at 5% and 10% YP at GET:YP ratios of 3:1 and 4:1 were produced with high encapsulation efficiency (>95%) and the reduction of YP concentration generated samples with similar flowability to YP-GET 1.1:1 at 15% YP.

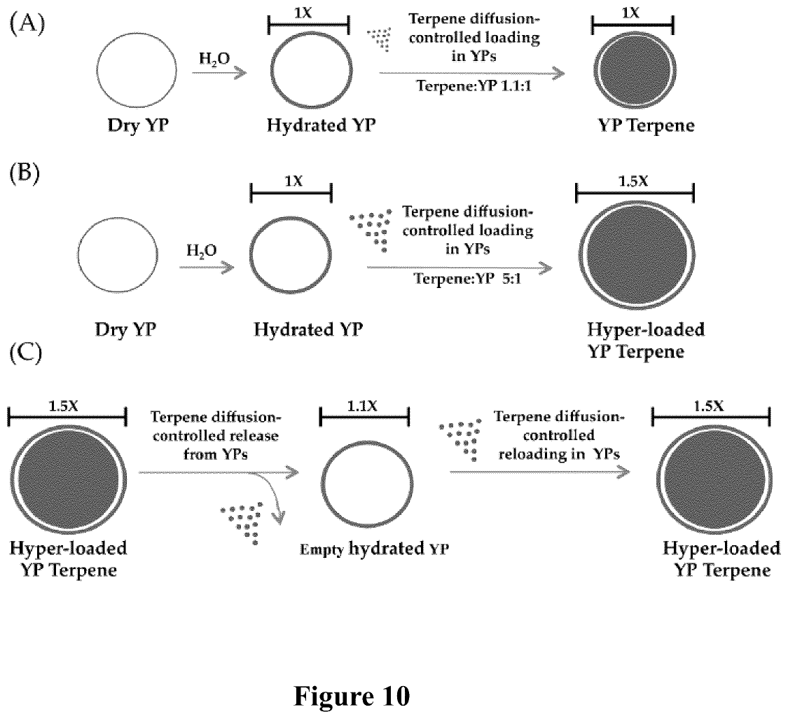

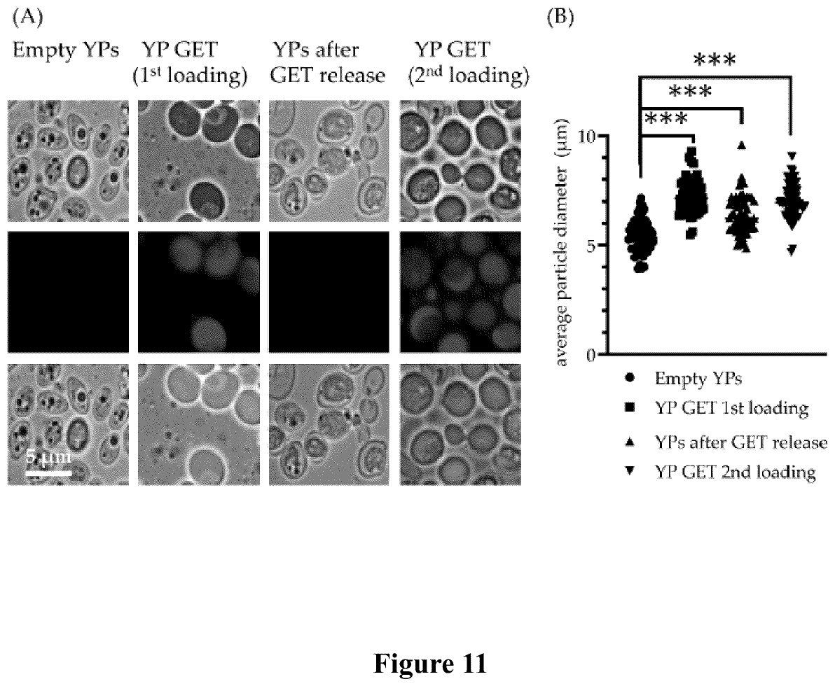

The optimized YP-GET samples with a composition of 10% YP and 30% GET were evaluated for GET release and the empty YP samples were loaded again with GET at the same ratio of 3:1. The schematic in FIG. 10C and microscopy images in FIG. 11 confirm good encapsulation of GET after first loading, empty YPs after complete release of GET in 5 cycles, and efficient encapsulation of GET after second loading. The YP diameter measurements in FIG. 11B show there was hysteresis following release of hyperloaded GET from YPs as there was only a partial reduction in average particle diameter (YPs after release of GET have an average diameter of 6.2 μm compared to the original YP average diameter of 5.4 μm).

### Biological Activity of Hyperloaded YP-GET

The antimicrobial biological activity of YP-GET samples was evaluated against different model microbial organisms to show that YP-GET retained the broad-spectrum antimicrobial effects of free GET. Terpenes have strong membrane permeation properties and a primary mode of action is the disruption in structural changes of the plasma membranes of both fungi and bacteria. The lipophilic isoprene unit of terpenes exhibits great affinity for the lipid portion of plasma membranes and the hydrophilic polar groups increase activity because of their interactions with proteins and carbohydrates.

### Antimicrobial Activity Assays Against Model Bacterial and Fungal Organisms

The antimicrobial activity of YP-GET was evaluated using a modified published microplate assay procedure (Sultanbawa, Y.; Cusack, A.; Currie, M.; Davis, C. An Innovative Microplate Assay to Facilitate the Detection of Antimicrobial 538Activity in Plant Extracts. J. Rapid Meth. Aut. Mic. 2009, 17 (4), 519-534. haps://doi.org/10.111/j.1745-4581.2009.00187.x). Samples of YP-GET were suspended in 100 μL of growth medium (LB 395was used in antibacterial assays and YPD in antifungal assays) and added to the first row 396 (Row A) of a 96-well plate (all wells in the 96-well plate contain additional 100 μL medium). Serial dilutions (1:1) were performed by transferring 100 μL from Row A to Row B, etc., and finally removing 100 μL from Row H. Diluted Escherichia coli Top10 (Invitrogen, Carlsbad CA), Staphylococcus aureus ATCC 19636 or Candida albicans SC5134 cells (100 μL, 106 cells/mL) were added to all wells of the plate. Initial (t=0) and final (t=16 h, 37° C.) absorbance readings were taken at 650 nm. The minimum inhibitory concentration (MIC) was determined as the concentration of terpene that inhibits bacterial or fungal growth as measured by absorbance by more than 75%.

All YP-GET samples were active against Escherichia coli, Staphylococcus aureus and Candida albicans and GET loading in YPs appears to enhance terpene minimum inhibitory concentration (MIC). Generally, unencapsulated GET emulsions are less stable and are 4-fold less potent than the YP encapsulated GET formulations. The E. coli and S. aureus bacteria required a much lower concentration of ampicillin, and C. albicans required a 10-fold lower concentration of fluconazole than YP GET to reach MIC 75%. However, resistance to ampicillin and fluconazole is common, whereas attempts to isolate strains resistant to 10-fold higher concentrations of the GET monoterpenoids have repeatedly failed. Further, ampicillin-resistant and fluconazole-resistant bacterial and fungal strain susceptibility to YP-GET remained the same as the sensitive strains showing the value of using monoterpenoids as a biocide.

TABLE 8
In vitro antimicrobial activity of negative YP control, YP-GET,
unencapsulated GET, and positive drug controls (ampicillin
and fluconazole) on two bacteria and one fungal model organisms.
The MIC results (mg/ml) represent the average of three biological
replicate experiments with three technical replicates for each
experiment. Statistically significant results were obtained
between YP-GET 1.1:1 and unencapsulated GET for the tested
microbes (* p<0.1, *** p<0.001, **** p<0.0001) and
between YP GET (all ratios) and ampicillin or fluconazole.

| | Minimum inhibitory concentration (MIC 75%) | | |
|---|---|---|---|
| Sample | E. coli | S. aureus | C. albicans |
| Empty YPs | Not active | Not active | Not active |
| YP-GET 1.1:1 | 0.156 ± 0.05 | 0.313 ± 0 | 0.156 ± 0.026 |
| YP-GET 2:1 | 0.156 ± 0.06 | 0.313 ± 0 | 0.156 ± 0.026 |
| YP-GET 3:1 | 0.156 ± 0.05 | 0.313 ± 0 | 0.156 ± 0.026 |
| YP-GET 4:1 | 0.156 ± 0.03 | 0.313 ± 0 | 0.156 ± 0.039 |
| YP-GET 5:1 | 0.156 ± 0.05 | 0.313 ± 0 | 0.156 ± 0.030 |
| Unencapsulated GET | 0.625 ± 0 **** | 1.250 ± 0.318* | 0.625 ± 0*** |
| Ampicillin | 0.008 **** | <0.00025 **** | — |
| Fluconazole | — | — | <2 **** |

### Example 6: Loading of Limonene Payload into Yeast Particle without a Solvent

### YP Loading of Limonene

The chemical and physical properties of limonene are shown in Table 9.

TABLE 9
Chemical and physical properties of limonene.

| | water/octanol | | |
|---|---|---|---|
| | partition | | Solubility |
| Structure | coefficient (log P) | Melting Point | in Water |
| 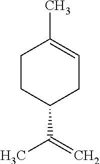 | 4.6 | −74° C. | 7.57 μg/mL |

Dry YPs from two different manufacturers were mixed with water (0.5mL water/g YP) for 30 minutes to obtain a uniform hydrated YP suspension. Limonene was added to the YP sample and incubated at 40° C. for one week to allow for complete limonene loading. Loading efficiency was quantitatively measured by the basic HPLC procedure described above using assay details listed in Table 10 and qualitatively assessed by fluorescence microscopy as described before. Particle diameter was measured as described before.

TABLE 10
HPLC assay conditions for quantitative analysis of limonene.
HPLC method for quantification of limonene

| | |
|---|---|
| Column | Waters Symmetry ® C18 3.5 μm 4.6 × 150 mm |
| Mobile Phase | 70% acetonitrile - 30% water |
| Flow rate | 1 mL/min |
| Injection volume | 10 μL |
| Running time | 10 minutes |
| Detection | Absorbance @ 210 nm |
| Retention time | 8.1 minutes |
| Linear range | 0-0.5 mg limonene/mL |

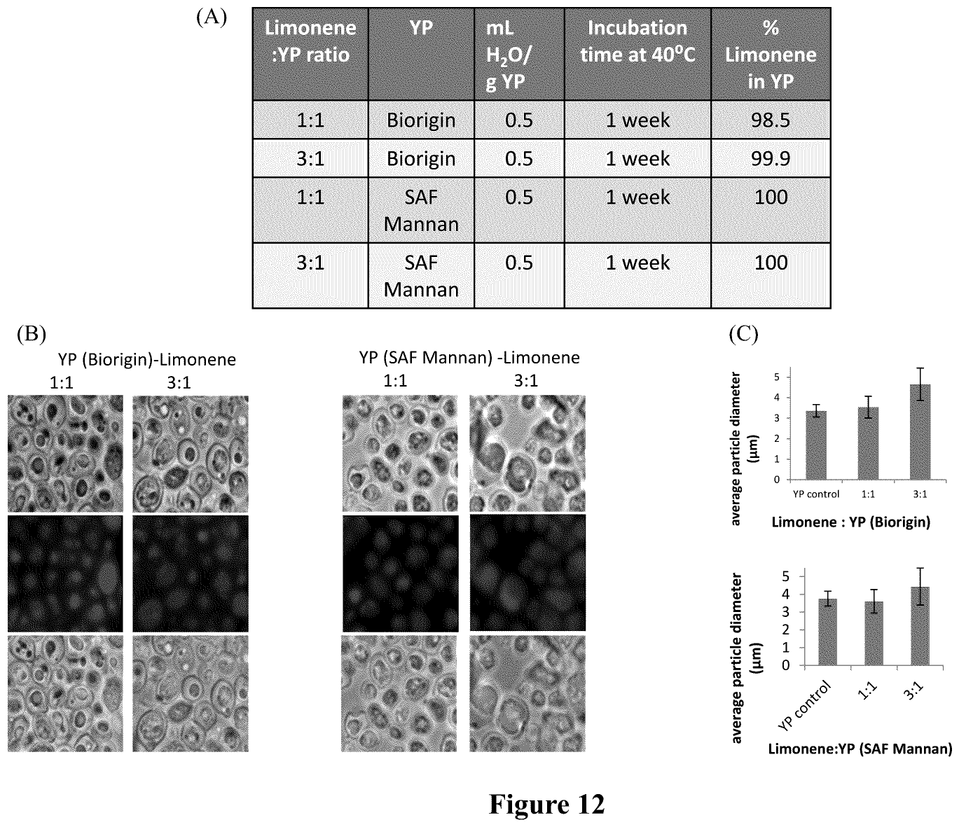

Due to its high water/octanol partition coefficient (log P), limonene loading was slow taking one week to complete. However, limonene was loaded at a ratio of limonene:YP ratio of 3:1 at high efficiency using both type of YPs (FIG. 12A). Light and fluorescent photomicrographs (FIG. 12B) showed that limonene was encapsulated in YPs and particle size measurements showed that hyperloading of limonene swelled YPs.

### Example 7: Loading of Clomazone Payload into Yeast Particle without a Solvent

### YP Loading of Clomazone

Clomazone is used as an herbicide. The chemical and physical properties of clomazone are shown in Table 11.

TABLE 11
Chemical and physical properties of clomazone.

| | water/ | | | |
|---|---|---|---|---|
| | octanol | | | |
| | partition | | | |
| | coefficient | Melt- | | |
| | (log | ing | Den- | Solubility |
| Structure | P) | Point | sity | in Water |
| 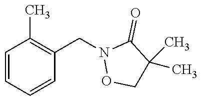 [SMILES: Cc1ccccc1CN1OCC(C)(C)C1=O] | 2.5 | 25° C. | 1.192 g/mL | 1.1 mg/mL |

Dry YPs from two different manufacturers were mixed with water (0.5 to 2.5 mL water/g YP) for 30 minutes to obtain a uniform hydrated YP suspension. Clomazone was added to the YP sample and incubated at 23° C. for 48 hours to allow for complete loading. Loading efficiency was quantitatively measured by the basic HPLC procedure described above using assay details listed in Table 12 and qualitatively assessed by fluorescence microscopy as described above. Particle diameter was measured as described above.

TABLE 12
HPLC assay conditions for quantitative analysis of clomazone.
HPLC method for quantification of clomazone

| | |
|---|---|
| Column | Waters Symmetry ® C18 3.5 μm 4.6 × 150 mm |
| Mobile Phase | 50% acetonitrile - 50% water |
| Flow rate | 1 mL/min |
| Injection volume | 10 μL |
| Running time | 8 minutes |
| Detection | Absorbance @ 210 nm |
| Retention time | 4.7-4.8 minutes |
| Linear range | 0-0.5 mg clomazone/mL |

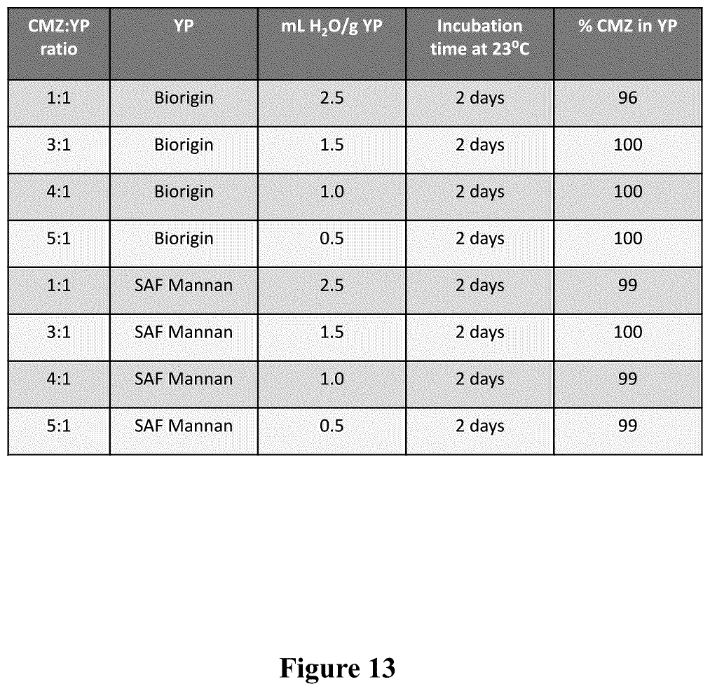

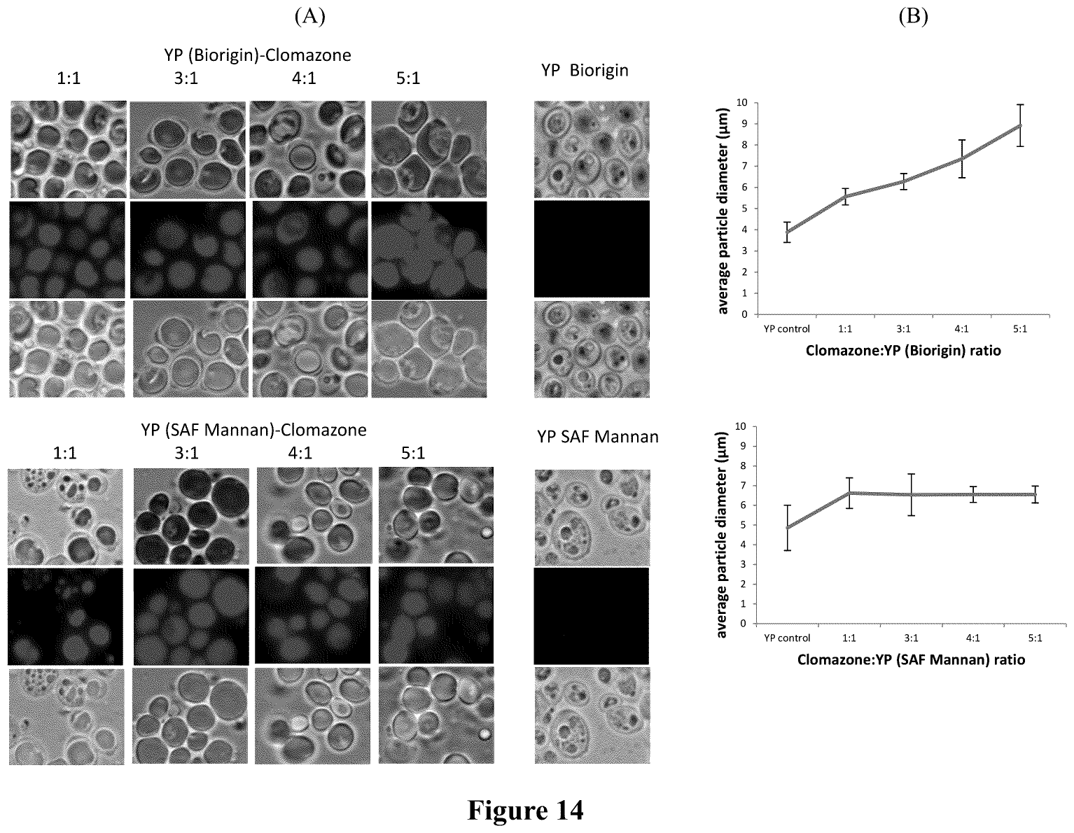

Clomazone was efficiently encapsulated in YPs up to w/w ratios of 5:1 with 96-100% of the clomazone located inside YPs (FIG. 13). Light and fluorescent photomicrographs (FIG. 14A) corroborated that clomazone was encapsulated within YPs and particle size measurements showed that hyperloading of limonene swelled YPs.

### YP-Clomazone Storage Temperature Stability

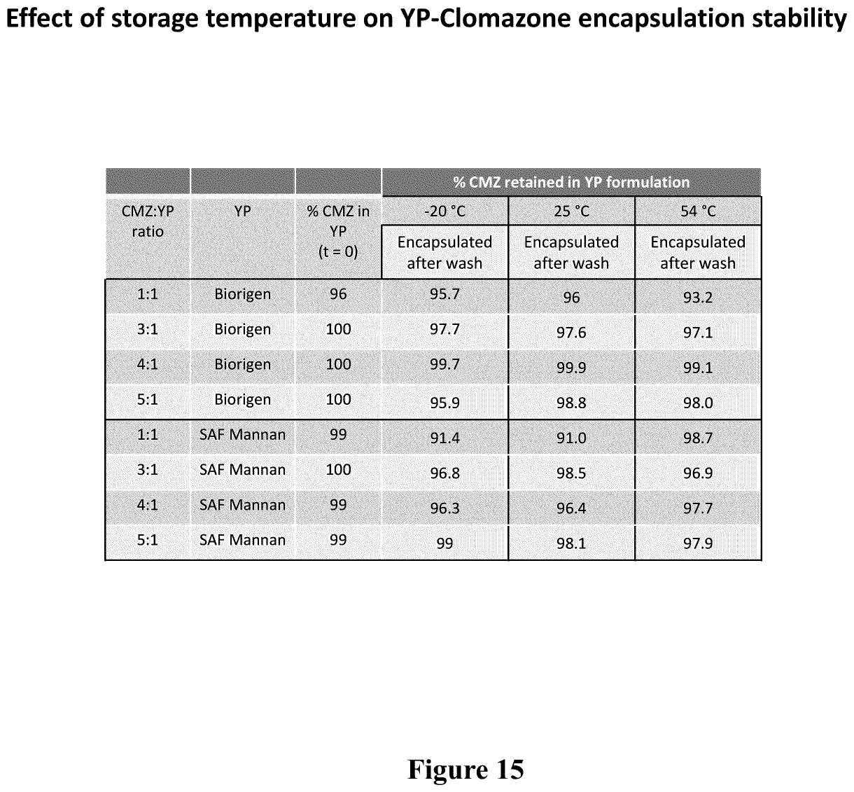

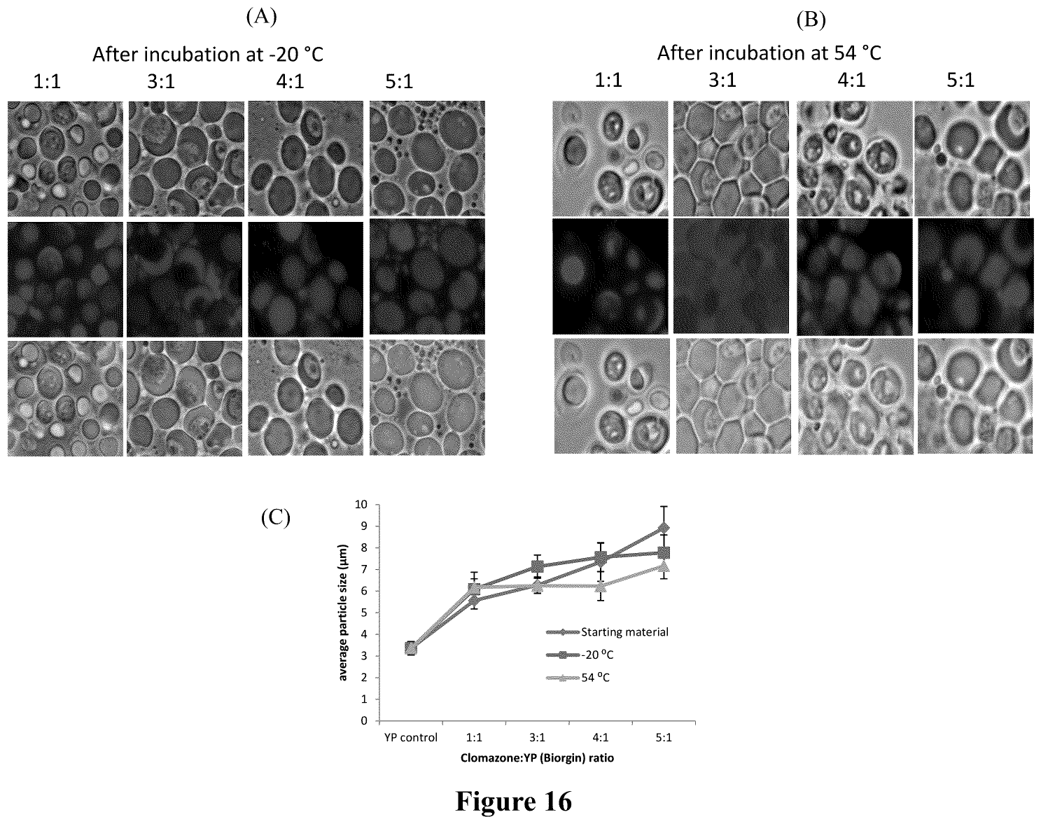

YP-Clomazone samples were diluted in water at a concentration of 150 g YP/L. One set of samples was subjected to three freeze (−20° C.)/thaw (25° C.) cycles (16-20 hours per freezing step, 8-10 hours thawing step). Two other sets of samples were stored at 25 or at 54° C. for two weeks. Samples were evaluated by microscopy for particle count and possible presence of broken particles. YP-clomazone samples were centrifuged and the free clomazone supernatant was collected. YP-clomazone pellets were diluted in water to a concentration of 10 mg clomazone/mL. Samples were centrifuged and supernatant was collected to measure free clomazone by HPLC. Light and fluorescent microscopy was performed on samples as described before. No significant amount of free clomazone was collected in supernatant after storage at various temperatures. Washing samples did not remove clomazone at concentrations above the maximum solubility of clomazone in water. FIG. 15 shows that the clomazone payload was retained in the YPs after storage. Clomazone samples were stable at all tested storage temperatures. FIGS. 16A and 16B show that clomazone was encapsulated within YPs after temperature stress. FIG. 16C shows that temperature stress did not affect YP particle. Taken together, data shows that YP-clomazone were storage stable.

### Example 8: Loading of Triallate Payload into Yeast Particle without a Solvent

### YP Loading of Triallate

Triallate is used as a pre-emergent herbicide. The chemical and physical properties of clomazone are shown in Table 13.

TABLE 13
Chemical and physical properties of triallate.

| | water/octanol | | | |
|---|---|---|---|---|
| | partition | | | |
| | coefficient (log | Melting | | Solubility |
| Structure | P) | Point | Density | in Water |
| 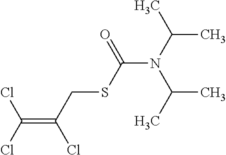 [SMILES: CC(C)N(C(=O)SCC(Cl)=C(Cl)Cl)C(C)C] | 4.6 | 34° C. | 1.27 g/mL | 4 μg/mL |

Dry YPs from two different manufacturers were mixed with water (2.5 to 5 mL water/g YP) for 30 minutes to obtain a uniform hydrated YP suspension. Triallate was added to the YP sample and incubated at 40° C. for 3 to 7 days to allow for complete loading. Loading efficiency was quantitatively measured by the basic HPLC procedure described above using assay details listed in Table 14 and qualitatively assessed by fluorescence microscopy as described before. Particle diameter was measured as described before.

TABLE 14
HPLC assay conditions for quantitative analysis of triallate.
HPLC method for quantification of triallate

| | |
|---|---|
| Column | Waters Symmetry ® C18 3.5 μm 4.6 × 150 mm |
| Mobile Phase | 90% acetonitrile - 10% water |
| Flow rate | 1 mL/min |
| Injection volume | 10 μL |
| Running time | 6.5 minutes |
| Detection | Absorbance @ 210 nm |
| Retention time | 3.8 minutes |
| Linear range | 0-500 μg triallate/mL |

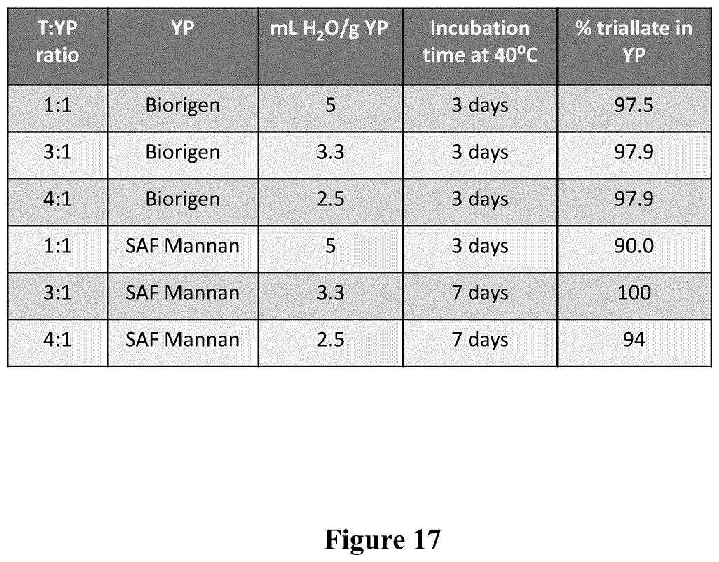

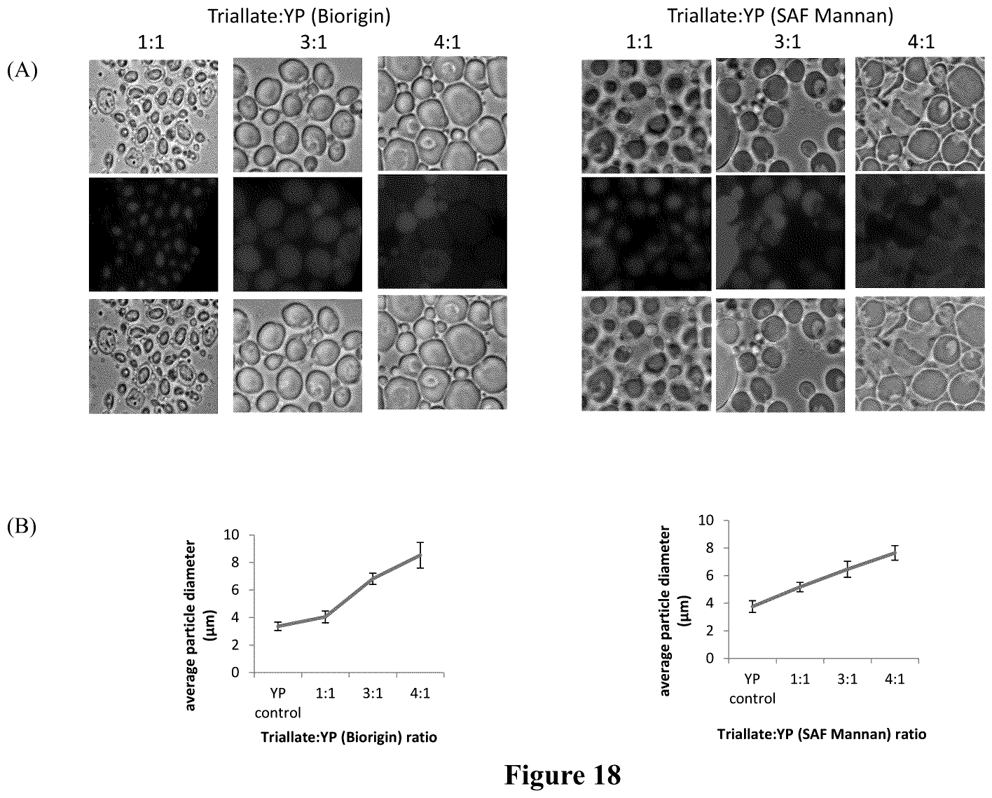

Triallate was efficiently encapsulated in YPs up to w/w ratios of 4:1 with 94 -100% of the triallate located inside YPs (FIG. 17). Light and fluorescent photomicrographs (FIG. 18A) corroborated that triallate was encapsulated within YPs and particle size measurements confirmed that hyperloading of triallate swelled YPs.

### YP-Triallate Storage Temperature Stability

YP-triallate samples were diluted in water at a concentration of 150 g YP/L. One set of samples was subjected to three freeze (−20° C.)/thaw (25° C.) cycles (16-20 hours per freezing step, 8-10 hours thawing step). Two other sets of samples were stored at 25 or at 54° C. for two weeks. Samples were evaluated by microscopy for particle count and possible presence of broken particles. YP-triallate samples were centrifuged, and the free triallate supernatant was collected. YP-triallate pellets were diluted in water to a concentration of 10 mg triallate/mL. Samples were centrifuged and supernatant was removed. YP-triallate pellets were resuspended in 90% ethanol and incubated at room temperature for 48H to extract encapsulated triallate. Encapsulated triallate was quantified by HPLC. Light and fluorescent microscopy was performed on samples as described before.

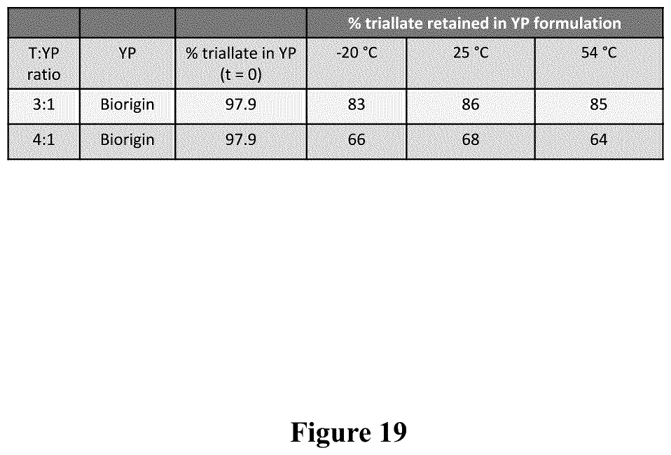

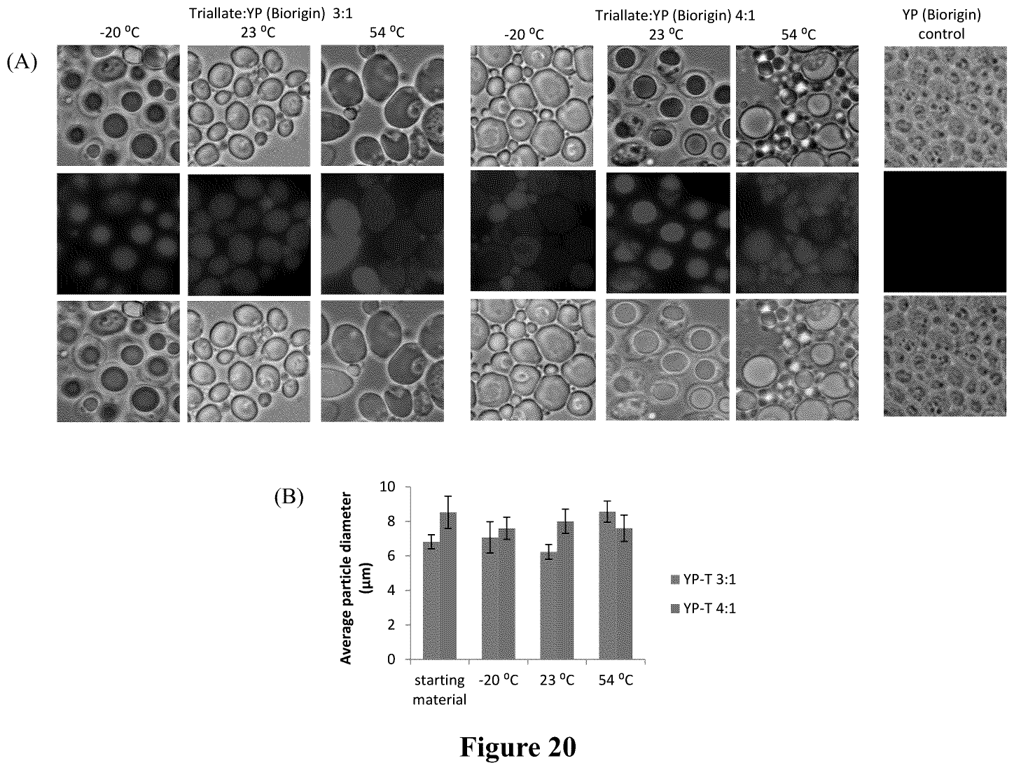

YPs with triallate:YP ratio of 3:1 were moderately stable at all temperatures with >80% triallate retained in the particles (FIG. 19). YP-Triallate 4:1 sample was less stable at all temperatures. Temperature stress did not affect encapsulation stability, as evidenced by fluorescence microcopy which showed that triallate remained inside YPs after (FIG. and particle diameter (FIG. 20B).

YP-Triallate storage stability could be improved by lowering particle concentration and triallate:YP ratio (100 g YP/L, 3.5:1 Triallate:YP)

### Effect of High Shear Stress on YP-Triallate Encapsulation Stability

YP-triallate samples were diluted in water at a concentration of 150 g YP/L. Samples were sonicated one or five times for 30 seconds per sonication cycle. Samples were evaluated by microscopy for particle count and possible presence of high number of broken particles. YP-triallate samples were centrifuged and the free triallate (bottom triallate oil layer and top triallate-water emulsion) was collected and quantified by HPLC. Samples were centrifuged and supernatant was removed. YP-triallate pellets were resuspended in 90% ethanol and incubated at room temperature for 48 h to extract encapsulated triallate. Encapsulated triallate was quantified by HPLC.

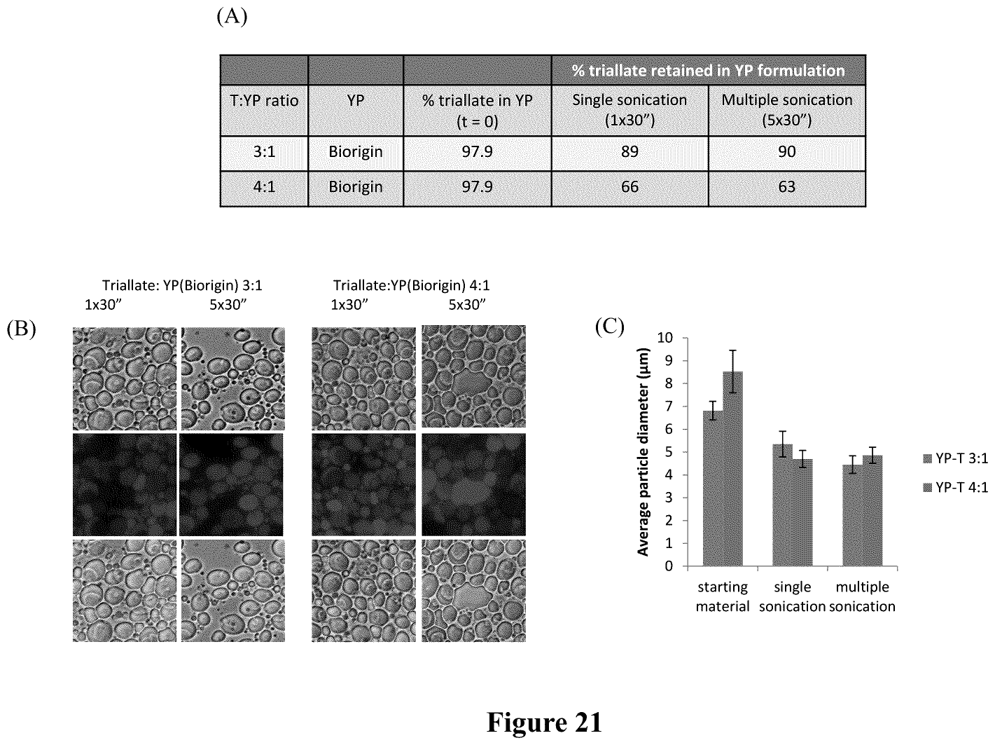

FIG. 21A shows that YPs with triallate:YP ratio of 3:1 showed high encapsulation stability during shear stress with 90% of the triallate remaining inside the YP. Light and fluorescent photomicrographs shown in FIG. 21B confirmed that the triallate payload remained within the YP after sonication. Average particle diameter decreased only slightly after sonication (FIG. 21C).

### Triallate Release Kinetics

Sustained payload release from YPs occurs by payload diffusion out of the particles and is a function of the payload's solubility in water. YP-triallate samples were diluted in water at target triallate concentrations of 0.001, 0.01, 0.1 and 1 mg/mL. Samples were incubated at room temperature. Amount of triallate released from the particles into the supernatant was quantified by HPLC as described before.

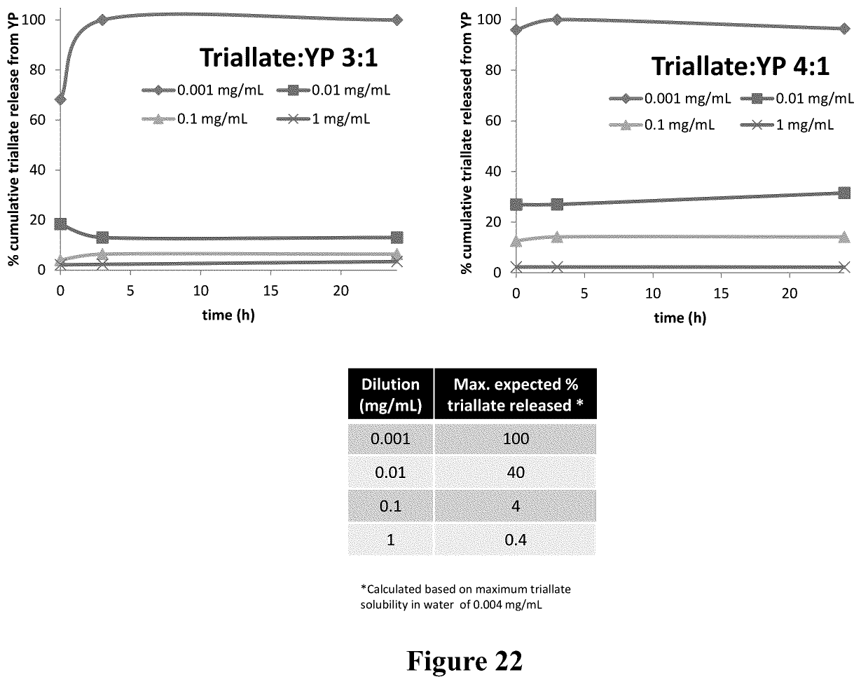

The results in FIGS. 22A and 22B show dilution to 0.001 mg/mL yields nearly 100% release of triallate from the YP.

### Example 9: Loading of Tetrahydrocannabinol (THC) Payload into Yeast Particle without a Solvent

### YP Loading of THC

The chemical and physical properties of are shown in Table 15.

TABLE 15
Chemical and physical properties of THC.

| | water/octanol | | |
|---|---|---|---|
| | partition | | |
| | coefficient (log | Melting | Solubility |
| Structure | P) | Point | in Water |
| 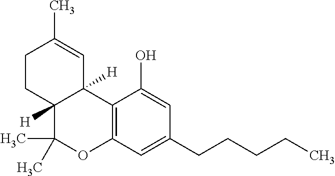 [SMILES: CCCCCc1cc(O)c2c(c1)OC(C)(C)[C@@H]1CCC(C)=C[C@@H]21] | 5.9 | <25° C. | 2.8 μg/mL |

Dry GLPs/YPs were mixed with water (0.5 to 4.5 mL water g GLP/YP) for 30 minutes to obtain a uniform hydrated YP suspension. THC was added to the GLP/YP sample and incubated at 23° C. for 48 hours to allow for complete THC loading. Loading efficiency was quantitatively measured by the basic HPLC procedure described above using assay details listed in Table 16 and qualitatively assessed by fluorescence microscopy as described above. Particle diameter was measured as described above.

TABLE 16
HPLC assay conditions for quantitative analysis of THC.
HPLC method for quantification of THC

| | |
|---|---|
| Column | Waters Symmetry ® C18 3.5 μm 4.6 × 150 mm |
| Mobile Phase | 70% acetonitrile - 30% water |
| Flow rate | 1 mL/min |
| Injection volume | 10 μL |
| Running time | 14 minutes |
| Detection | Absorbance @ 208 nm |
| Retention time | 10.2 minutes |
| Linear range | 0-250 μg THC/mL |

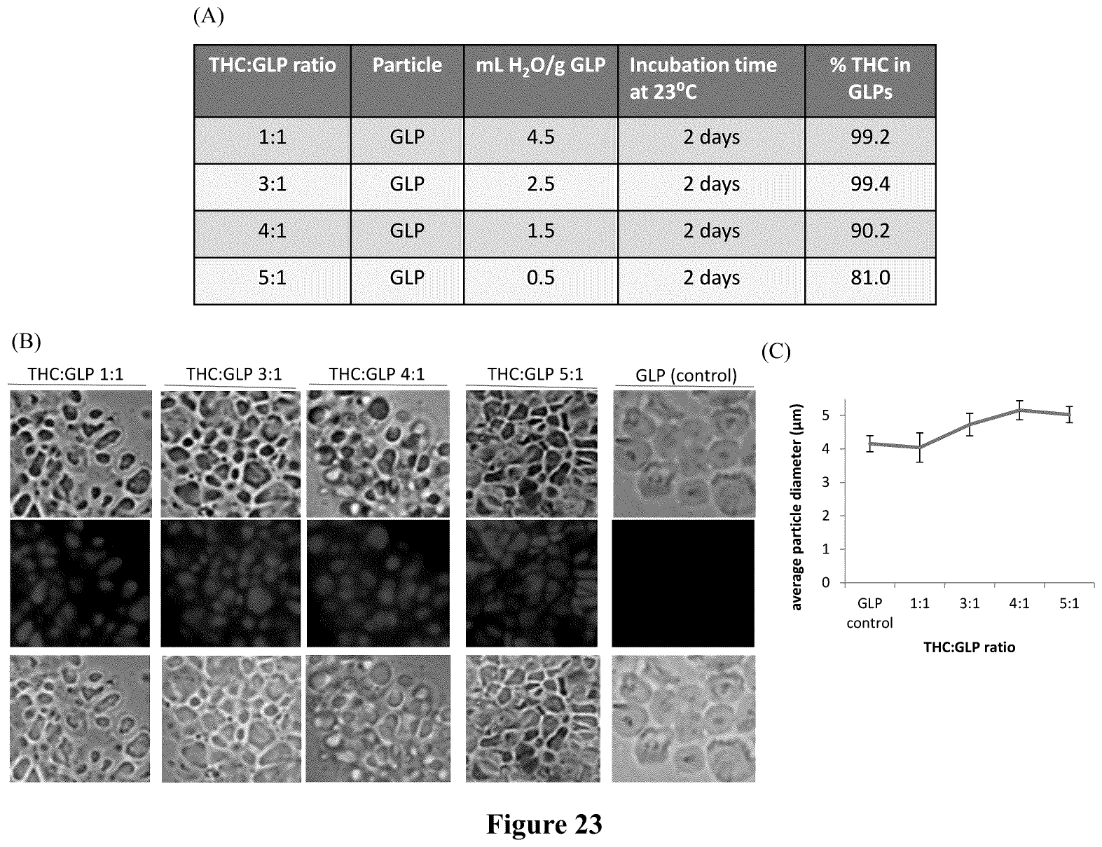

THC was loaded at a ratio of THC:YP ratio of up to 5:1 at high efficiency (FIG. 23A). Light and fluorescent photomicrographs (FIG. 23B) confirmed that THC was encapsulated in GLP/YPs and particle size measurements (FIG. 23C) showed that hyperloading of THC swelled YPs.

### Example 10: Loading of α-Cyhalotrin (λCy) Payload into Yeast Particle without a Solvent

### YP Loading of λCy

The chemical and physical properties of are shown in Table 17.

TABLE 17
Chemical and physical properties of λCy.

| | water/octanol | | | |
|---|---|---|---|---|
| | partition | | | |
| | coefficient | | | Solubility |
| Structure | (log P) | Melting Point | Density | in Water |
| 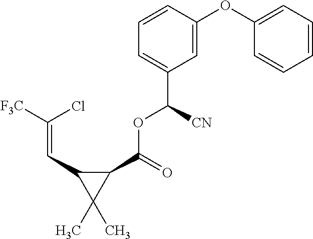 | 7.0 | 49.2° C. | 1.3 g/mL | 2.8 μg/mL |

Two different kinds of dry YPs were mixed with water (0.5 to 4.5 mL water/g YP) for 30 minutes to obtain a uniform hydrated YP suspension. λCy was added to the YP sample and incubated at 55° C. for one week to allow for complete λCy loading. Loading efficiency was quantitatively measured by the basic HPLC procedure described above using assay details listed in Table 18 and qualitatively assessed by fluorescence microscopy as described before. Particle diameter was measured as described before.

TABLE 18
HPLC assay conditions for quantitative analysis of λCy.
HPLC method for quantification of λCy

| | |
|---|---|
| Column | Waters Symmetry ® C18 3.5 μm 4.6 × 150 mm |
| Mobile Phase | 70% acetonitrile - 30% water |
| Flow rate | 1 mL/min |
| Injection volume | 10 μL |
| Running time | 16 minutes |
| Detection | Absorbance @ 210 nm |
| Retention time | 11.8-12.0 minutes |
| Linear range | 0-0.4 mg λCy/mL |

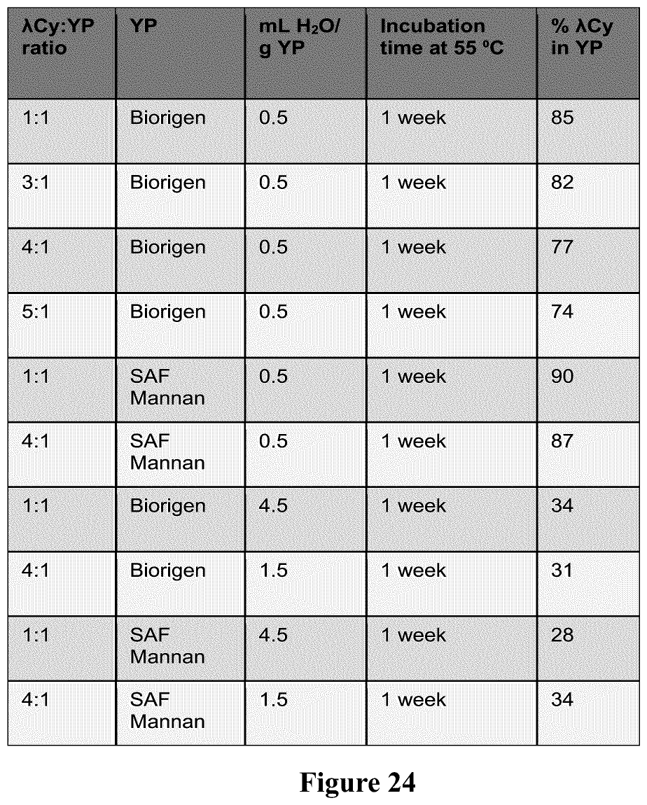

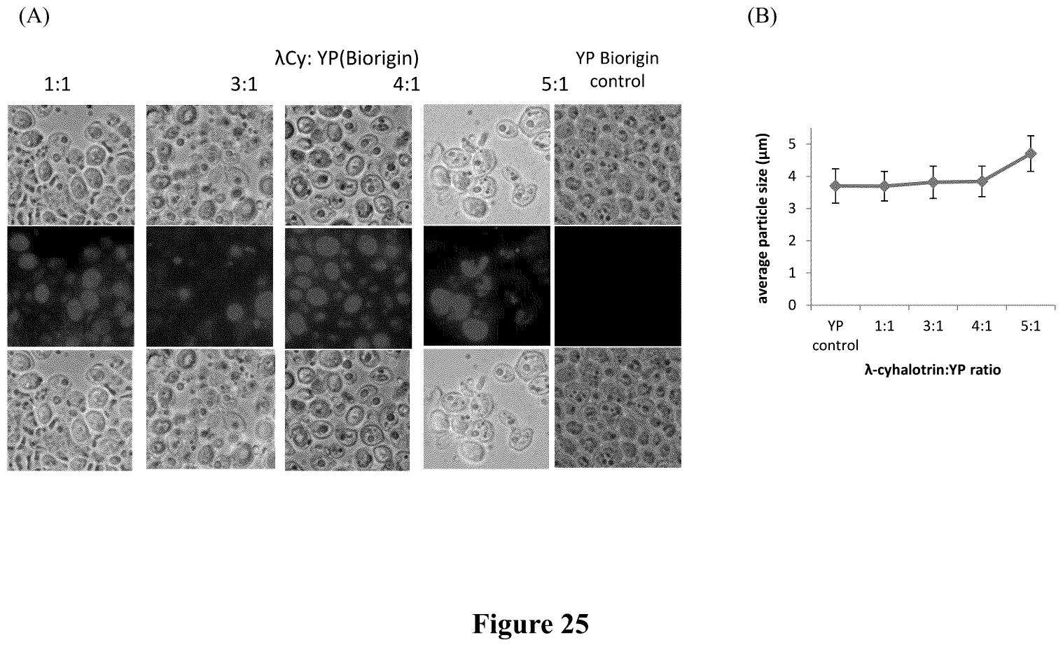

λCy is extremely hydrophobic and thus efficient loading was only possible when YPs were hydrated with less than 0.5 mL water/g YP. FIG. 24 shows that encapsulation efficiency λCy at ratios of λCy:YP ratio of up to 5:1 was higher when YPs were hydrated with minimum amount of water. Light and fluorescent photomicrographs (FIG. 25A) confirmed that λCy was encapsulated in YPs and particle size measurements (FIG. 25B) showed that hyperloading of λCy only slightly swelled YPs likely due to low water content of YP, lower payload encapsulation (77-90%) and high density of λCy (1.3 g/mL).

### λCy Release Kinetics

As λCy was extremely water insoluble, it did not release from YP-λCy in aqueous suspensions. Addition of a surfactant (IGEPAL) to the YP-λCy suspension significantly improved λCy release.

λCy-YP samples loaded at a w/w λCy:YP ratio of 1:1 were diluted in water ±surfactant (1% w/v IGEPAL) at target λCy concentrations of 0.001, 0.01, 0.1 and 1 mg/mL. Samples were incubated at room temperature for 3 hours. Supernatant was collected to measure amount of λCy released from the particles. The amount of λCy was quantified by HPLC as described above.

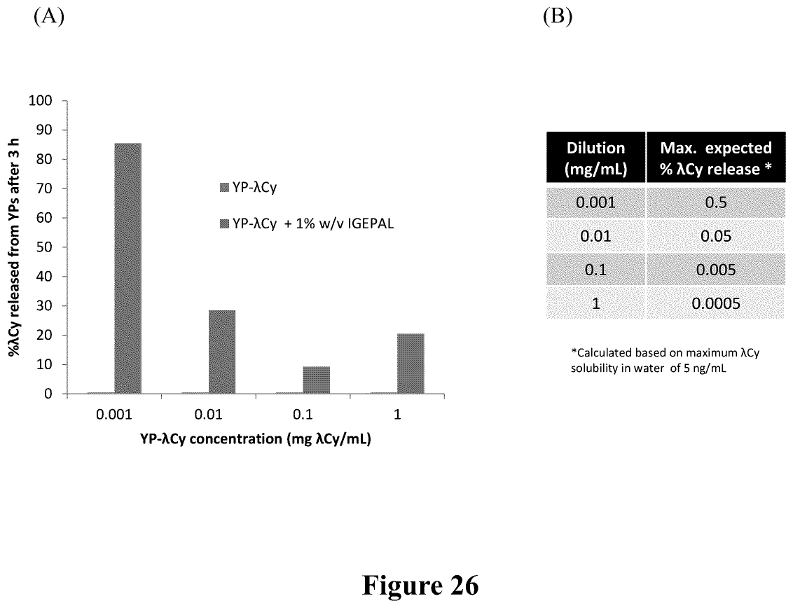

FIG. 26 shows the expected and actual λCy at various dilutions. Dilution to 0.001 mg λCy/mL yielded over 80% release of from YPs.

### Example 11: Loading of Payload into Yeast Particle with an Organic Solvent

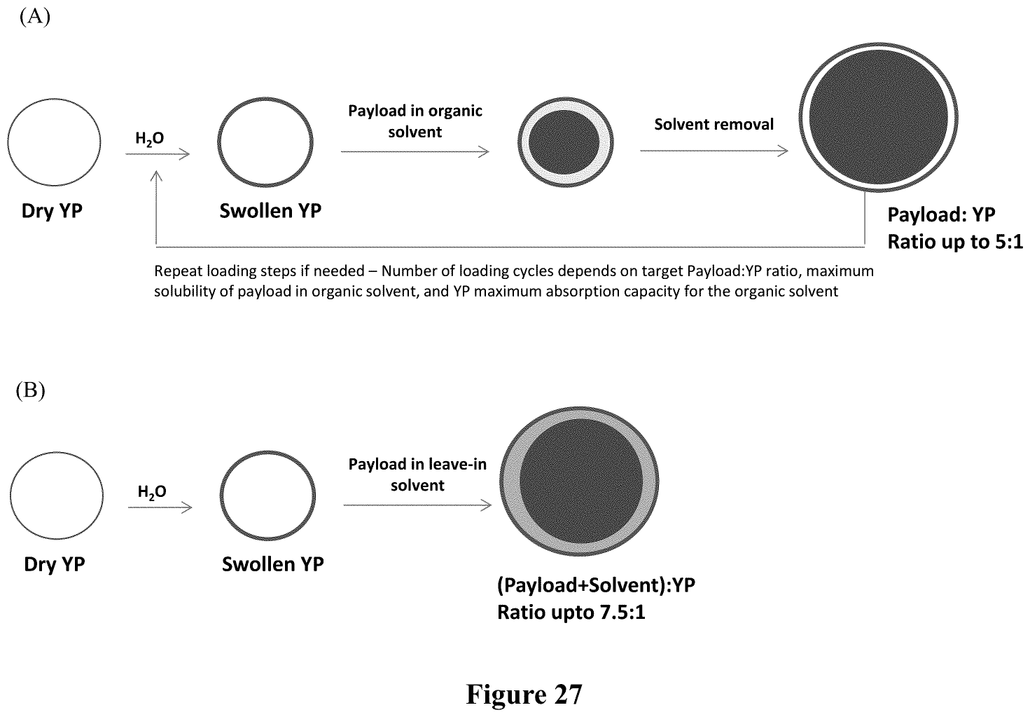

Payloads that have low water solubility or are insoluble in water and have a melting point >70° C. could be loaded into YP with the aid of an organic solvent. Loading could be achieved in a single step and a loading capacity of YP up to 5:1 payload:YP weight ratio could be achieved. Multiple loading cycles could be required for complete loading of payload with low solubility in the organic solvent. Solubility of >1 g payload per mL organic solvent was desirable to minimize number of loading cycles. Minimum amount of water required to swell particles is 0.5 μl of water/mg YP. The kinetics of loading depended on YP lipid content, the amount of water used to swell particles (0.5-5 μl/mg YP), and the solubility of payload in the organic solvent used for loading. Organic solvents that posed potential safety issues could be removed completely after payload is loaded in the YP. FIG. 27A shows the schematic of loading payloads into YPs using organic solvents.

### YP Loading of Penthiopyrad (PTP) Using Acetone

Penthiopyrad is a carboxamide fungicide used to control a broad spectrum of diseases on large variety of crops. The structure and solubility of PTP in various organic solvents are shown in Table 19.

TABLE 19
Structure and solubility of PTP in various organic solvents.

| | | | PTP Solubility | |
|---|---|---|---|---|
| | | | (mg/mL) | |
| Structure | Acetone | Dichloromethane | Ethyl Acetate | Methanol |
| 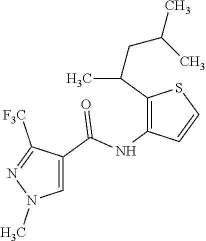 [SMILES: CC(C)CC(C)c1sccc1NC(=O)c1cn(C)nc1C(F)(F)F] | 557 | 431 | 349 | 402 |

PTP is highly soluble in acetone and dichloromethane. Acetone was chosen as the loading solvent as it is a better solvent than dichloromethane because it is safe and can be removed by washing loaded YP-PTP samples with water. Dry YPs were mixed with water (0.5 water/g YP) for 30 minutes to obtain a uniform hydrated YP suspension. PTP dissolved in acetone was added to the YP sample and incubated at 23° C. for 24 hours to allow PTP loading into YP. The loading cycle was repeated to achieve higher weight ratios of PTP:YP. The loading efficiency was quantitatively measured by the basic HPLC procedure described above using assay details listed in Table 20 and qualitatively assessed by fluorescence microscopy as described above. Particle diameter was measured as described above.

TABLE 20
HPLC assay conditions for quantitative analysis of PTP.
HPLC method for quantification of PTP

| | |
|---|---|
| Column | Waters Symmetry ® C18 3.5 μm 4.6 × 150 mm |
| Mobile Phase | 70% acetonitrile - 30% water |
| Flow rate | 1 mL/min |
| Injection volume | 10 μL |
| Running time | 7 minutes |
| Detection | Absorbance @ 210 nm |
| Retention time | 4.0-4.1 minutes |
| Linear range | 0-500 μg PTP/mL |

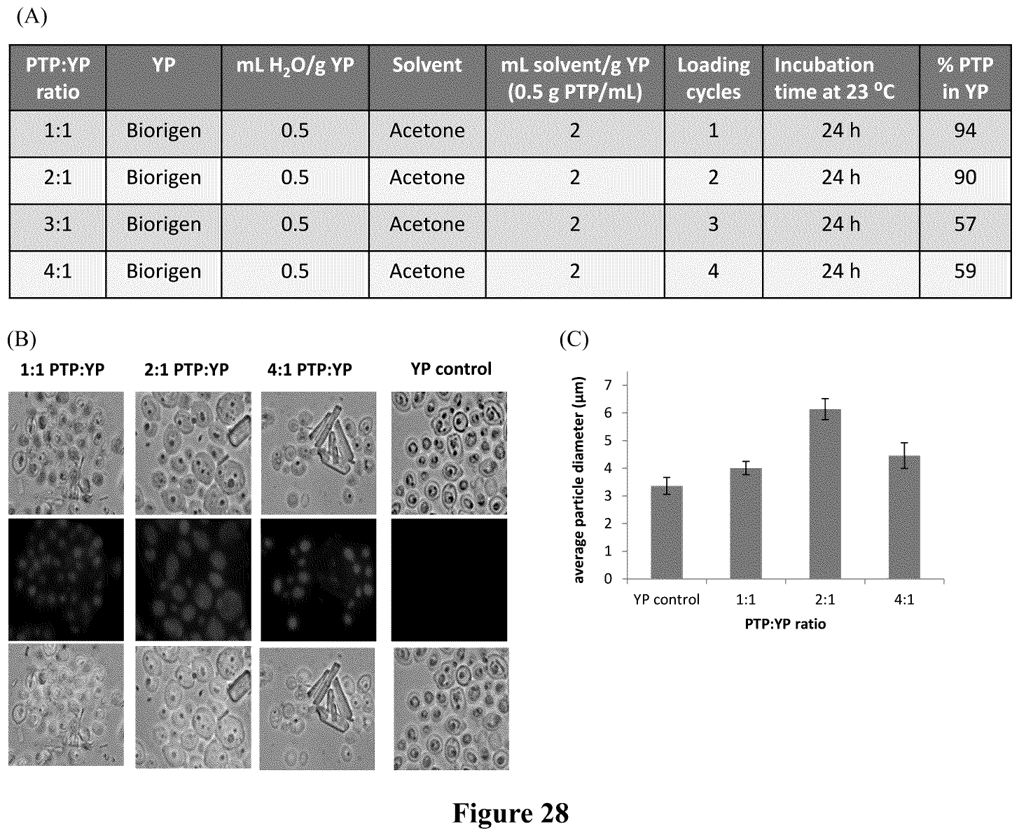

PTP was loaded at PTP:YP ratios of up to 4:1 with four loading cycles. Encapsulation diminished with subsequent loading cycles (FIG. 28A). Light and fluorescent photomicrographs (FIG. 28B) confirmed that PTP was encapsulated in YPs and particle size measurements (FIG. 28C) showed that hyperloading of PTP swelled YPs.

YP Loading of Prothioconazole (PRO) using Acetone

Prothioconazole is a triazolinthione fungicide used as a broad spectrum systemic fungicide. PRO is highly soluble in acetone, polyethene glycol and esters. Chemical and physical properties of prothioconazole are shown in Table 21.

TABLE 21
Chemical and physical properties of prothioconazole.

| Structure | | 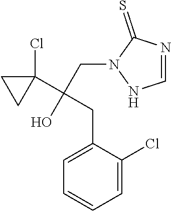 [SMILES: OC(Cc1ccccc1Cl)(Cn1[nH]cnc1=S)C1(Cl)CC1] | |
|---|---|---|---|
| Water/octanol partition coefficient (log P) | | 4.05 | |
| Melting point | | 139.1-144.5 C. | |
| Density | | 1.36 | g/mL |
| Solubility | Water | 22.5 | mg/L |
| | Acetone | 444 | mg/mL |
| | Ethyl acetate | 220 | mg/mL |
| | DMSO | 126 | mg/mL |

Acetone was chosen as the loading solvent because it is safe and can be removed by washing loaded YP-PRO samples with water. Loading was achieved though one or more loading cycles. Each loading cycle included the following steps: Dry YPs were mixed with water (0.5 water/g YP) to obtain a uniform hydrated YP suspension, samples were incubated overnight at 4° C. PRO dissolved in acetone was added to the YP sample and incubated at 23° C. for 24 hours to allow PRO loading into YP. Organic solvent and water were removed after each loading step and reintroduced prior to the next loading step to improve loading efficiency. The loading efficiency was quantitatively measured by the basic HPLC procedure described above using assay details listed in Table 22 and qualitatively assessed by fluorescence microscopy as described above.

TABLE 22
HPLC assay conditions for quantitative analysis of PRO.
HPLC method for quantification of prothioconazole

| | |
|---|---|
| Column | BDS Hypersil ® C18 5 μm 4.6 × 150 mm |
| Mobile Phase | 70% acetonitrile - 30% water |
| Flow rate | 0.8 mL/min |
| Injection volume | 30 μL |
| Running time | 8 minutes |
| Detection | Absorbance @ 210 nm |
| Retention time | 5.6 minutes |
| Linear range | 0-0.5 mg prothioconazole/mL |

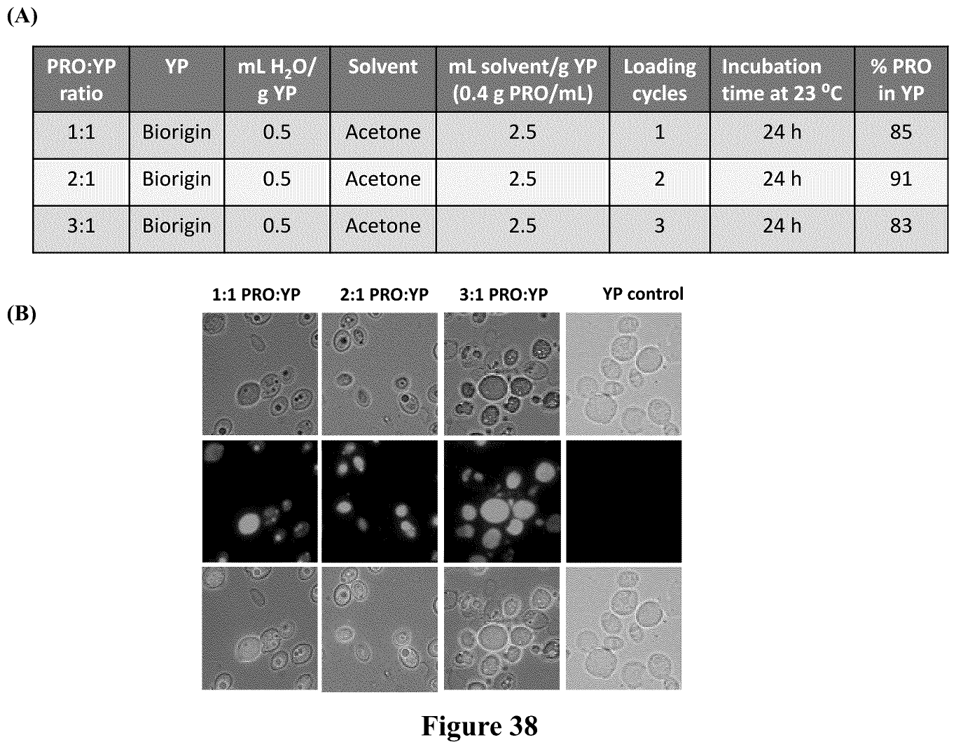

PRO was loaded at PRO:YP ratios of up to 3:1 with three loading cycles with a encapsulation efficiency of more than 83% (FIG. 38A). Light and fluorescent photomicrographs (FIG. 38B) confirmed that PTP was encapsulated in YPs.

### YP Loading of Cannabidiol (CBD) Using Ethanol

The structure and solubility of cannabidiol (CBD) in various organic solvents are shown in Table 23.

TABLE 23
Structure and solubility of CBD in various organic solvents.

| | | | CBD Solubility |
|---|---|---|---|
| | | | Dimethylsulfoxide |
| Structure | Ethanol | Methanol | (DMSO) |
| 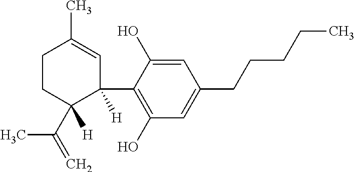 [SMILES: C=C(C)[C@@H]1CCC(C)=C[C@H]1c1c(O)cc(CCCCC)cc1O] | Miscible at 1:1 weight ratio* | Miscible at 1:1 weight ratio* | 200 mg/mL |

*volume of CBD solution = volume of CBD + volume of solvent

Dry GLPs were mixed with water (0.5 water/g GLP) for 30 minutes to obtain a uniform hydrated GLP suspension. CBD dissolved in acetone was added to the GLP sample and incubated at 23° C. for 48 hours to allow CBD loading into GLP. The loading cycle was repeated to achieve higher weight ratios of CBD:YP. The loading efficiency was quantitatively measured by the basic HPLC procedure described above using assay details listed in Table 24 and qualitatively assessed by fluorescence microscopy as described above. Particle diameter was measured as described above.

TABLE 24
HPLC assay conditions for quantitative analysis of CBD.
HPLC method for quantification of CBD

| | |
|---|---|
| Column | Waters Symmetry ® C18 3.5 μm 4.6 × 150 mm |
| Mobile Phase | 70% acetonitrile - 30% water |
| Flow rate | 1 mL/min |
| Injection volume | 10 μL |
| Running time | 7 minutes |
| Detection | Absorbance @ 208 nm |
| Retention time | 5.0-5.2 minutes |
| Linear range | 0-200 μg CBD/mL |

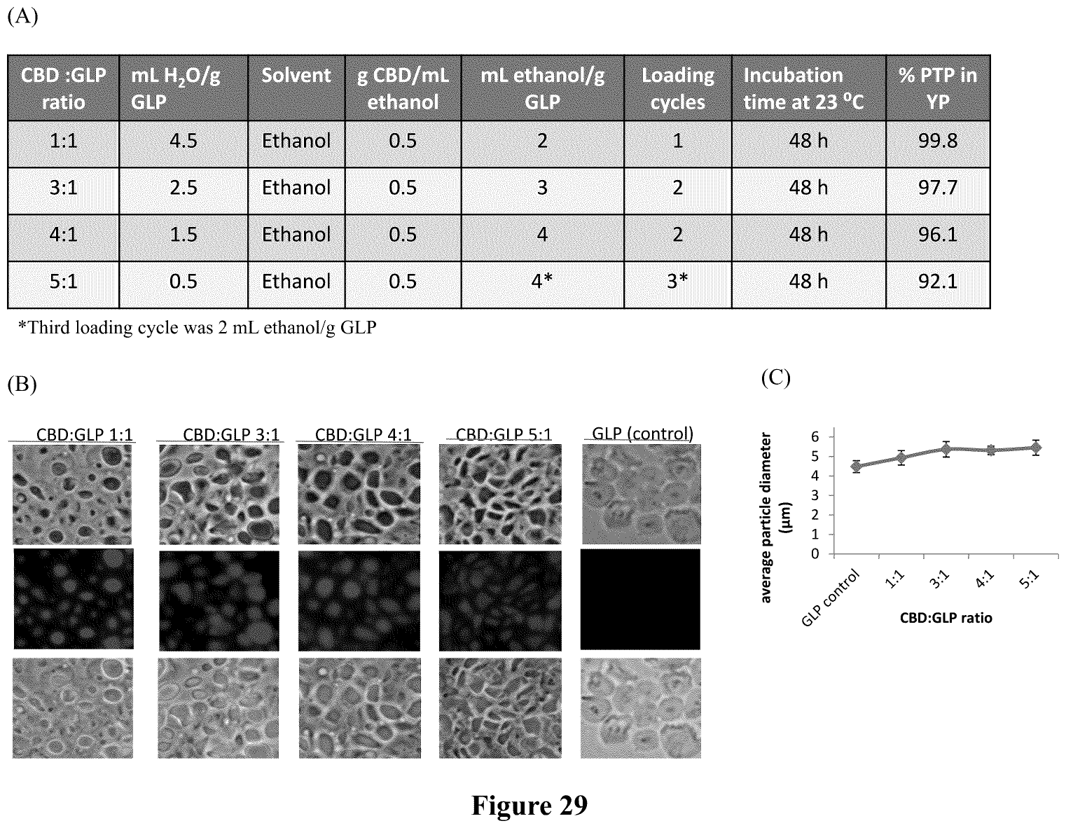

CBD was loaded with high encapsulation efficiency at CBD:YP weight ratios of up to 5:1 with multiple loading cycles (FIG. 29A). Light and fluorescent photomicrographs confirmed that CBD was encapsulated in GLPs (FIG. 29B) and particle size measurements (FIG. 28C) showed that hyperloading of CBD swelled YPs.

### Example 12: Loading of Payload into Yeast Particle with a Leave-in Organic Solvent

Payloads that have low water solubility or are insoluble in water and have a melting point >70° C. can be loaded into YP with the aid of an organic solvent. Solvents that are safe for the target application (e.g., pharmaceutical, agricultural) can be used for loading and allowed to remain in the YP as a “leave-in” solvent along with the payload.

Loading could be achieved in a single step and could yield loading capacity of up to 5:1 payload:YP weight ratio. A minimum amount of water required to swell particles is 0.5 μl of water/mg YP. The kinetics of loading depended on YP lipid content, amount of water used to swell particles (0.5-5 μl/mg YP), temperature, and the solubility of payload in the organic leave-in solvent used for loading. FIG. 27B shows a schematic of loading payloads into YPs using organic leave-in solvents. Use of safe, leave-in organic solvents eliminated the need of solvent removal. However, leave-in solvent added to the total amount of material loaded in particles, limiting maximum payload:YP loading capacity.

### YP Loading of Spinosad Using Leave-In Solvent GET (Mix of Geraniol-Eugenol-Thymol)

Spinosad is a natural substance made by a soil bacterium that can be toxic to insects that is used to control a wide variety of pests, including, but not limited to, thrips, leaf miners, spider mites, mosquitoes, ani, fruit flies and the like. Many products containing spinosad are used on crops and ornamental plants. The structures of the two main components of spinosad (spinosyn A and D) and spinosad solubility in organic solvent GET are shown in Table 25.

TABLE 25
Structures of main components and solubility of spinosad.

| Structure | | Spinosad Solubility |
|---|---|---|
| 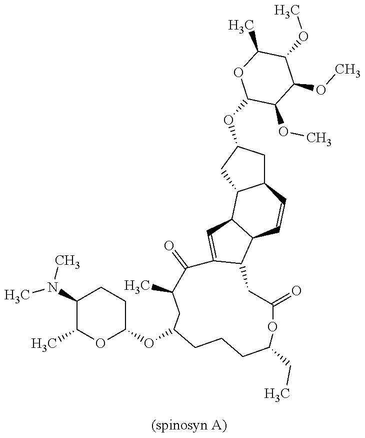 | 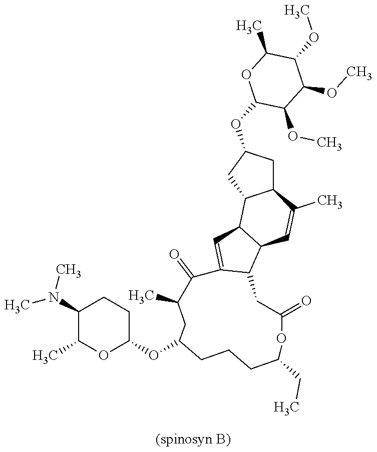 | Miscible 1:1 spinosad:GET weight ratio* |

*volume of spinosad solution = volume of spinosad + volume of solvent

Dry YPs were mixed with water (1.0 mL water/g YP) for 30 minutes to obtain a uniform hydrated YP suspension. Spinosad dissolved in GET424 (a mixture of geraniol (G), eugenol (E), thymol (T) at a weight ratio composition of 2:1:2) was added to the YP sample and incubated at 50° C. for 24 hours to allow spinosad loading into YP. Loading efficiency was quantitatively measured by the basic HPLC procedure described above using assay details listed in Table 26 and qualitatively assessed by fluorescence microscopy as described above. Particle diameter was measured as described above.

TABLE 26
HPLC assay conditions for quantitative analysis of spinosad.
HPLC method for quantification of spinosad

| | |
|---|---|
| Column | Waters Symmetry ® C18 3.5 μm 4.6 × 150 mm |
| Mobile Phase | 45% methanol, 45% acetonitrile, |
| | 10% aqueous sodium acetate (0.5% w/v) |
| Flow rate | 1 mL/min |
| Injection volume | 10 μL |
| Running time | 10 minutes |
| Detection | Absorbance @ 240 nm |
| Retention time | Spinosad - four peaks from 4.5 to 6.5 min |
| | Spinosad was quantified using the area |
| | of all four peaks. |
| Linear range | 0-500 μg spinosad/mL |

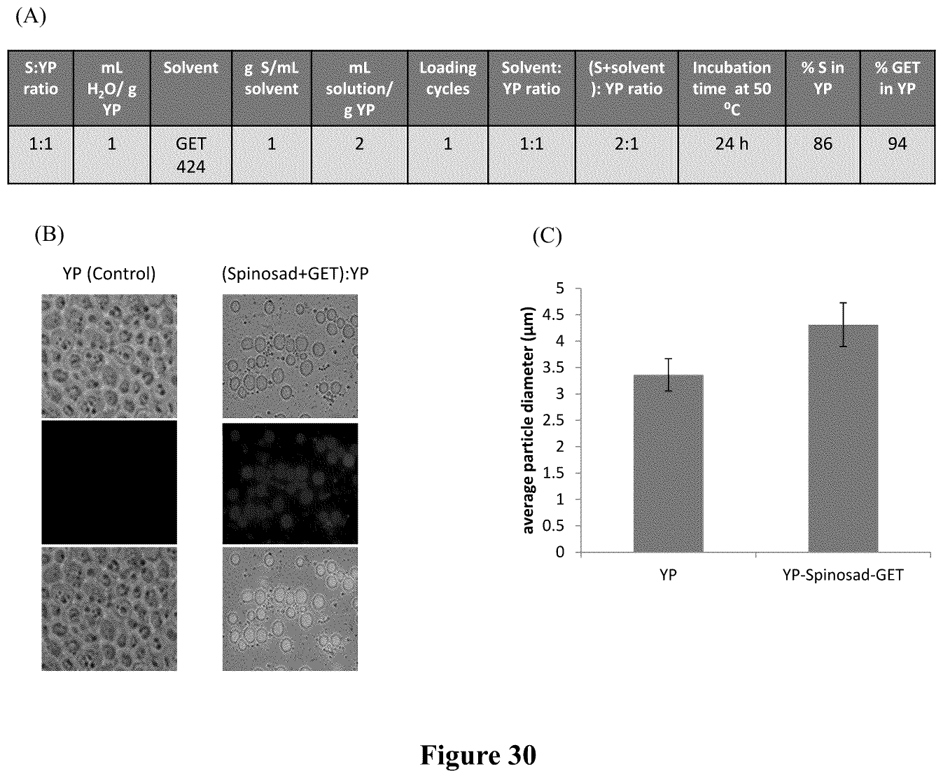

Spinosad solution in GET424 could be efficiently loaded in YP at a 2:1 (Spinosad+GET424):YP ratio with high encapsulation efficiency (FIG. 30A). Light and fluorescent photomicrographs (FIG. 30B) confirmed that spinosad-GET mixture was encapsulated in YPs and particle size measurements (FIG. 30C) showed that YPs swelled as a result of loading of payload and leave-in solvent.

### Spinosad Release Kinetics

(Spinosad-GET424)-YP samples loaded at a w/w (spinosad+GET424):YP ratio of 2:1 were diluted in water at target spinosad concentrations of 0.01, 0.1, 1.0 and 10 mg/mL. Samples were incubated at room temperature for 3 hours. The supernatant was collected to measure amount of spinosad released from the particles. Amount of spinosad was quantified by HPLC as described above.

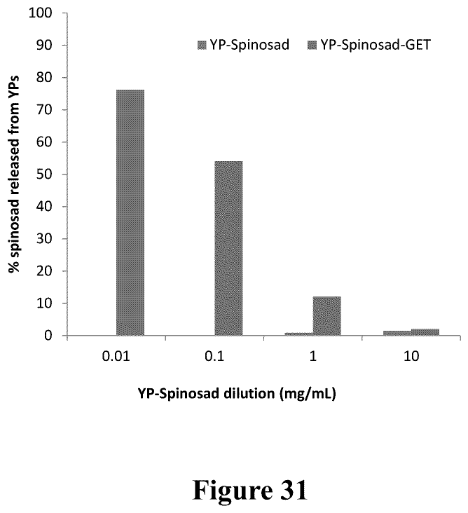

FIG. 31 shows that dilution to 0.01 mg spinosad/mL yielded over 70% release of from YPs. Control YPs containing spinosad without leave-in solvent GET424 did not release, indicating that GET424 improves spinosad release from YPs.

### Example 13: YP Loading of Penthiopyrad (PTP) Using Leave in Solvents GET (Mix of Geraniol-Eugenol-Thymol) or DMDA

The structure and solubility of PTP in organic leave-in solvents are shown in Table 27.

TABLE 27
Structure and solubility of PTP in various organic solvents.

| | | | PTP Solubility | N,N- |
|---|---|---|---|---|
| | | | (mg/mL) | dimethyldecanamide |
| Structure | Eugenol | Geraniol | GET-424* | (DMDA) |
| 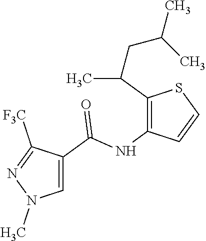 [SMILES: CC(C)CC(C)c1sccc1NC(=O)c1cn(C)nc1C(F)(F)F] | 200 | 200 | 200 | Miscible at 1:1 w/w ratio† |

*GET424: a mixture of geraniol (G), eugenol (E), thymol (T) at a weight ratio composition of 2:1:2
†volume of PTP solution = volume of PTP + volume of solvent

Dry YPs were mixed with water (0.5 mL water/g YP) for 30 minutes to obtain a uniform hydrated YP suspension. PTP dissolved in GET424 (a mixture of geraniol (G), eugenol (E), thymol (T) at a weight ratio composition of 2:1:2) or N,N-dimethyldecanamide (DMDA) was added to the YP sample and incubated at 23° C. for 24 hours (when GET424 was the leave-in solvent) or 48 hours (when DMDA was the leave-in solvent) to allow PTP loading into YP. Loading efficiency was quantitatively measured by the basic HPLC procedure described above using assay details listed in Table 20 and qualitatively assessed by fluorescence microscopy as described above. Particle diameter was measured as described above.

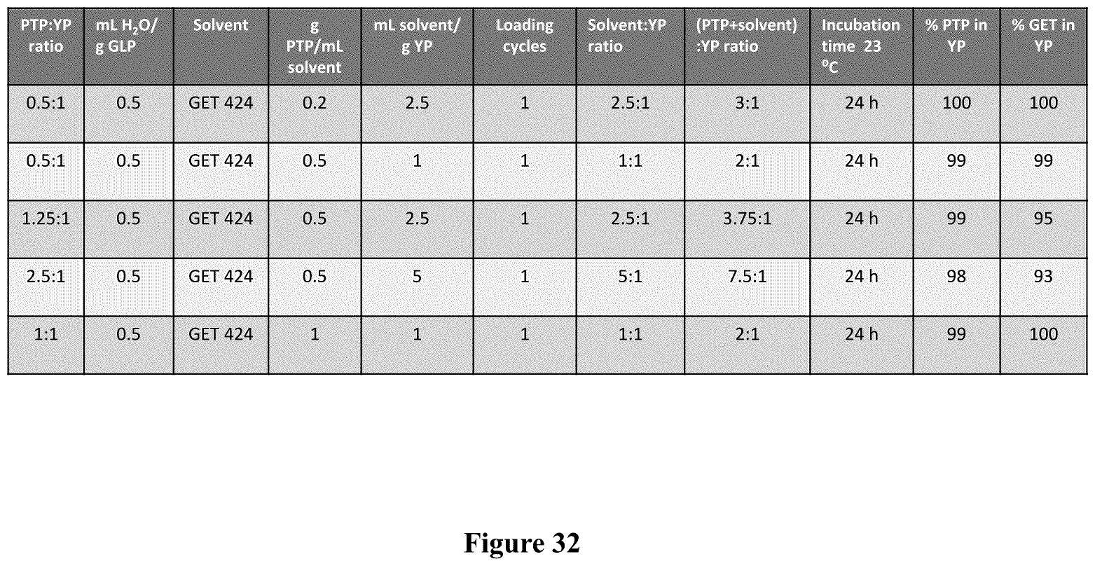

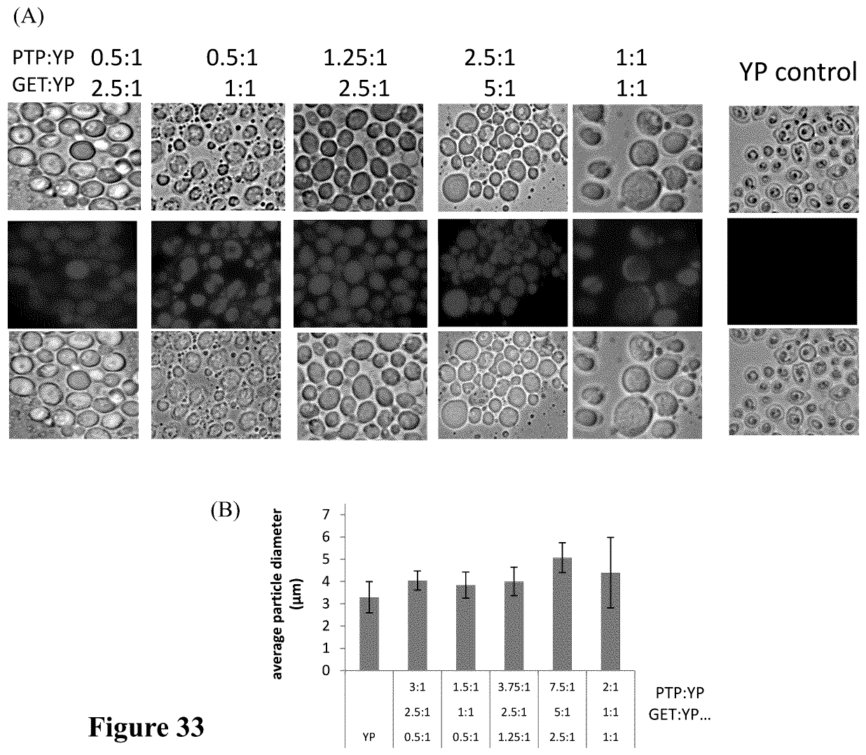

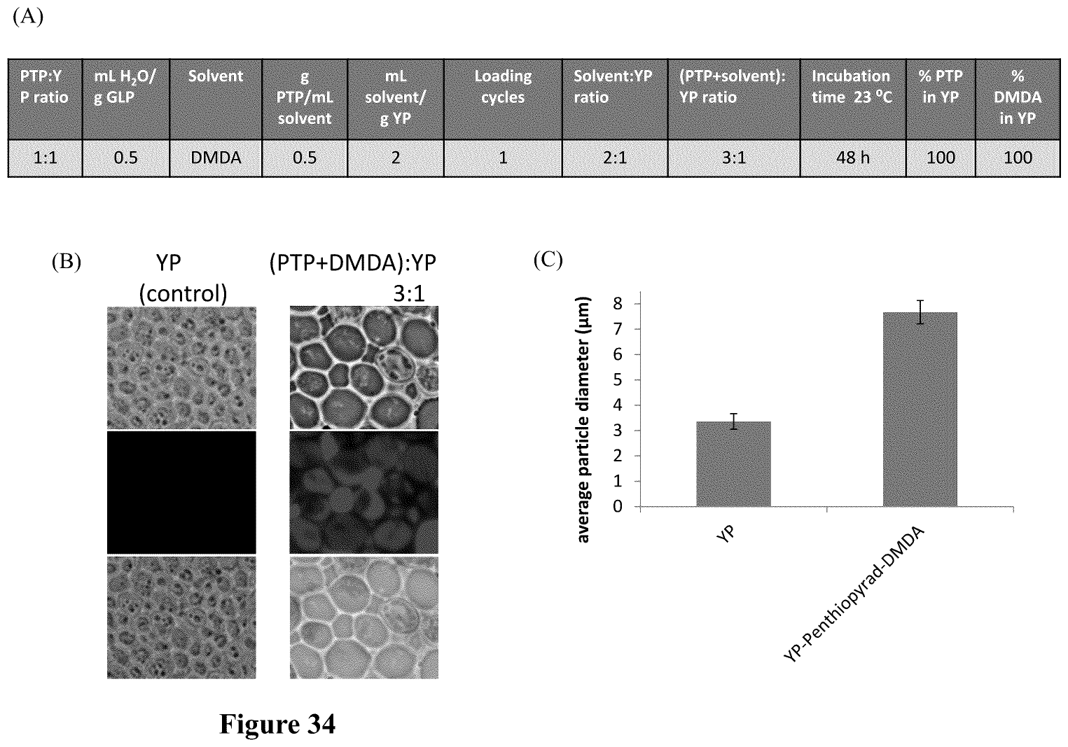

PTP was loaded at (PTP+solvent):YP ratios of up to 7.5:1 with GET424 (FIG. 32) and up to 3:1 with DMDA (FIG. 34A). Penthiopyrad could be efficiently encapsulated in YP using GET424 as solvent at a maximum PTP solubility in GET of 0.2 g/mL. However, loading was limited to a PTP:YP ratio of 0.5:1 and total (GET424+PTP):YP ratio of 3:1 (FIG. 32). Use of PTP supersaturated (0.5 and 1 g PTP/mL GET424) solutions increased encapsulation efficiency to a ratio of 2.5:1 PTP : YP and total (GET424+PTP):YP ratio of 7.5:1 (FIG. 32). Light and fluorescent photomicrographs confirmed that PTP-GET424 and PTP-DMDA were encapsulated in YPs (FIGS. 33A, 34B) and particle size measurements (FIGS. 33C, 34C) showed that hyperloading of PTP swelled YPs.

### PTP Release Kinetics

PTP-YP samples prepared without leave in solvent or with leave-in solvent GET424 or DMDA. Samples carrying PTP:YP weight ratio 1:1, PTP:GET:YP ratio of 1:1:1 or PTP:DMDA:YP ratio of 1:1:1 were diluted in water at a target PTP concentration of 0.001, 0.01, 0.1 and 1 mg/mL. Samples were incubated at room temperature. Supernatant was collected to measure amount of payload released from the particles.

PTP was loaded at a w/w (spinosad+GET424):YP ratio of 2:1 and samples were diluted in water at target PTP concentrations of 0.01, 0.1, 1.0 and 10 mg/mL. Samples were incubated at room temperature for 3 hours. Supernatant was collected to measure amount of PTP released from the particles. Amount of PTP was quantified by HPLC as described before.

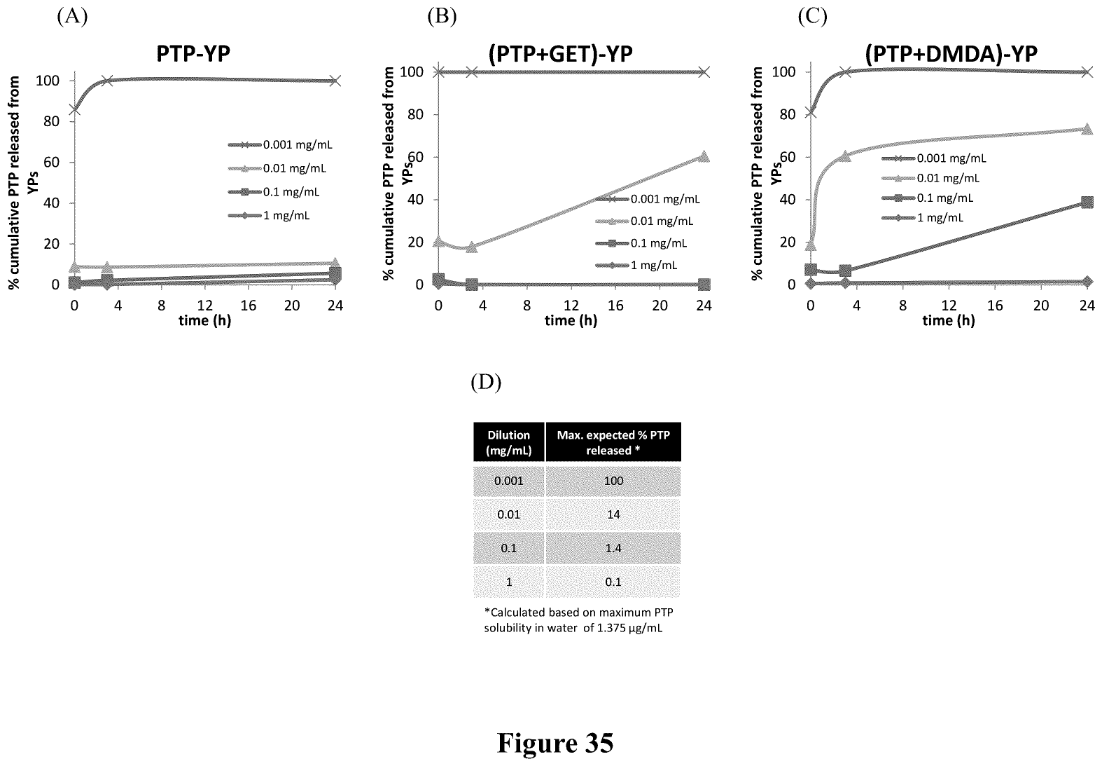

FIG. 35A shows that when PTP was encapsulated in YPs without a leave-in solvent, PTP released from YPs only at a concentration above maximum solubility in water. Leave-in solvents promoted more efficient release of the PTP payload from YP. FIG. 35B showed that terpene improved release at 0.01 mg PTP/mL. GET increased PTP release at concentrations 7 times higher than PTP maximum solubility in water. DMDA improved release at 0.1 and 0.01 mg PTP/mL. DMDA increased PTP release at concentrations 7 to 70 times higher than PTP maximum solubility in water (1.375 ug/mL) (FIG. 35C) and thus, was determined to be a better leave-in solvent for PTP.

### Example 14: YP Loading of Cannabidiol (CBD) Using Leave in Solvent Octanoic Acid (OA)

The structure and solubility of cannabidiol (CBD) in organic leave-in solvents are shown in Table 28.

TABLE 28
Structure and solubility of CBD in various organic leave-in solvents.

| | CBD Solubility | |
|---|---|---|
| | (mg/mL) | |
| Solvent | Octanoic Acid | |
| Structure | (OA) | GET424* |
| 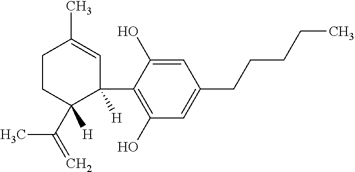 [SMILES: C=C(C)[C@@H]1CCC(C)=C[C@H]1c1c(O)cc(CCCCC)cc1O] | Miscible at 0.5:1 CBD:OA ratio | 10 mg/mL |

*GET424: a mixture of geraniol (G), eugenol (E), thymol (T) at a weight ratio composition of 2:1:2

Dry GLPs were mixed with water (1.5 mL water/g GLP) for 30 minutes to obtain a uniform hydrated YP suspension. CBD dissolved in octanoic acid was added to the GLP sample and incubated at 23° C. for 48 hours to allow CBD loading into YP. Loading efficiency was quantitatively measured by the basic HPLC procedure described above using assay details listed in Table 24 and qualitatively assessed by fluorescence microscopy as described above. Particle diameter was measured as described above.

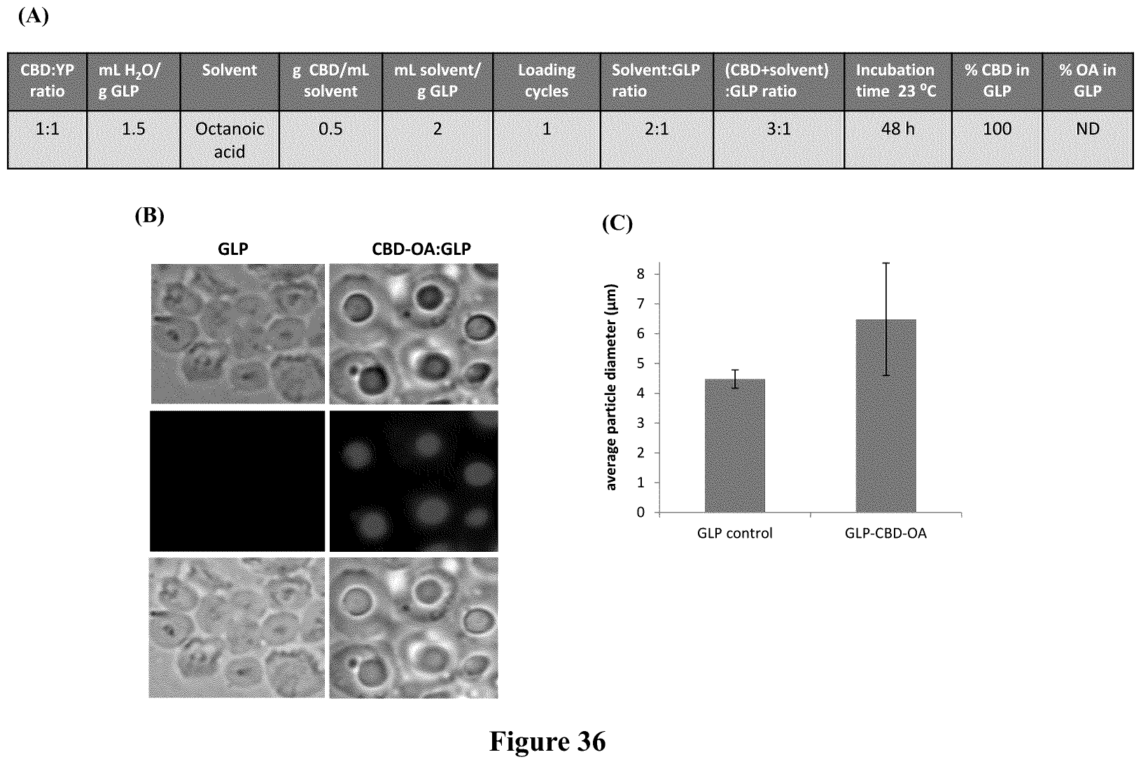

Cannabidiol could be loaded efficiently with OA as leave-in solvent in GLPs at a 3:1 (CBD+OA):GLP ratio with high encapsulation efficiency (FIG. 36A). Light and fluorescent photomicrographs (FIG. 36B) confirmed that CBD-OA mixture was encapsulated in GLPs and particle size measurements (FIG. 36C) showed that YPs swelled as a result of loading of CBD and OA.

### Example 15: Loading Payloads that are Liquid or Oil at Temperatures Below 70° C.

Payloads that are liquid or oil at temperatures below 70° C., for example fish oil, can be loaded in YP without using an organic solvent.

YP samples were hydrated overnight at 4° C. with 0.5 μL water/mg YP. Fish oil was added to the particles to yield a fish oil:YP ratio of 1:1, 2:1 or 3:1. The mixture was incubated 48 hours at room temperature. Samples were suspended in water (10 mg YP/mL) and visually evaluated for presence of an emulsion in the supernatant (unencapsulated fish oil). Samples were centrifuged, the supernatant was collected, and the pellet was suspended in 1 mL of water (10 mg YP/mL). YP encapsulated fish oil was quantified by spectrophotometric measurement (530 nm) of the fatty acid components of fish oil with phosphovanillin. Fish oil encapsulated within YPs was visualized by staining YPs with Nile Red, a stain that detects lipid droplets.

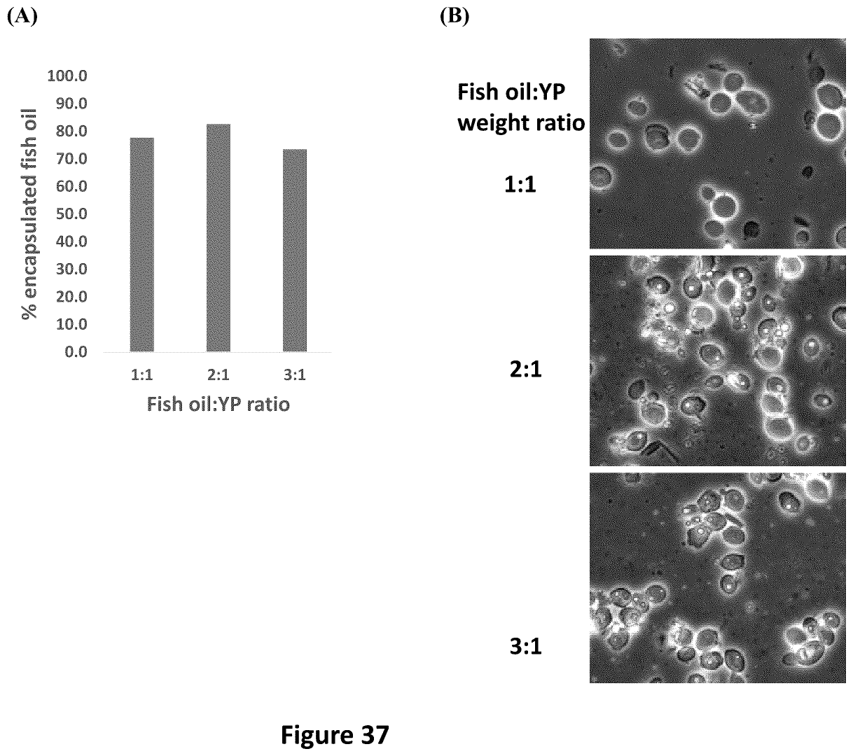

FIG. 37A shows the results of spectrophotometric quantification of encapsulated fish oil. For all ratios tested, the above procedure yielded more than 70% encapsulation of fish oil within YPs. FIG. 37B shows fluorescent photomicrographs of Nile Red stained YPs. The particle size of loaded YPs was 5.6±0.7 nm when the fish oil:YP ratio was 1:1 and 6.7 ±1.2 nm when the ratio was 3:1.

### LITERATURE CITED

Calo, J. R.; Crandall, P. G.; O′Bryan, C. A.; Ricke S. C. Essential Oils as Antimicrobials in Food Systems- A Review. Food Control. 460 2015, 54, 111-119.
Bakry, A. M.; Abbas, S.; Ali, B.; Majeed, H.; Abouelwafa, M. Y.; Mousa, A.; Liang, L. Microencapsulation of Oils: A Comprehen-462sive Review of Benefits, Techniques, and Applications. Compr. Rev. Food Sci. Food Saf. 2016, 15, 143-182. 463.
Bhalerao, Y. P.; Wagh S. J. A Review on Thymol Encapsulation and its Controlled Release through Biodegradable Polymer Shells. 465Int. J. Pharm. Sci. 2018, 2, 4522-4532. doi: 10.13040/IIPSR.0975-8232.9(11).4522-32 466.
Gómez, B.; Barba, F. J.; Domínguez, R.; Putnik, P.; Bursać Kovačević, D.; Pateiro, M.; Toldrá, F.; Lorenzo, J. M. Microencapsula-467tion of Antioxidant Compounds through Innovative Technologies and Its Specific Application in Meat Processing. Trends Food 468Sci. Technol. 2018, 82, 135-147.
Saifullah, M.; Shishir, M.; Ferdowsi, R.; Rahman, M.; Van Vuong, Q. Micro and Nano Encapsulation, Retention and Controlled 470Release of Flavor and Aroma Compounds: A Critical Review. Trends Food Sci. Technol. 2019, 86, 230-251. 471.

1. A hyperloaded yeast particle (YP) comprising a YP and a hydrophobic payload, wherein:
the hydrophobic payload is present within the YP;
the weight by weight (w/w) ratio of the hydrophobic payload:the hyperloaded YP is between about 2:1 and about 5:1; and
the hydrophobic payload is releasable from the hyperloaded YP upon contact with an aqueous solution.

2. The hyperloaded YP of claim 1, wherein the YP is selected from the group consisting of a Biorigin YP, an SAF Mannan YP, a yeast cell wall particle (YCWP), a glucan particle (GP) and a mixture thereof, optionally wherein the GP is selected from the group consisting of a yeast glucan particle (YGP), a yeast glucan-mannan particle (YGMP), a glucan lipid particle (GLP), a whole glucan particle (WGP) and a mixture thereof.

3. (canceled)

4. The hyperloaded YP of claim 1, wherein the hydrophobic payload comprises one or more hydrophobic compounds, optionally wherein the hydrophobic payload is dissolved in an organic solvent that is optionally selected from the group consisting of acetone, dichloromethane, ethyl acetate, ethanol, methanol, dimethyl sulfoxide (DMSO), eugenol, geraniol, a mixture of geraniol (G), eugenol (E), thymol (T) at a weight ratio composition of 2:1:2 (GET424), N,N-dimethyl-decanamide (DMDA), octanoic acid, lauric acid, undecanoic acid, glycofurol, vitamin E, fatty acid, and medium chain triglyceride (MCT).

5. (canceled)

6. (canceled)

7. The hyperloaded YP of claim 4, wherein the organic solvent remains as a leave-in solvent in the hyperloaded YP.

8. The hyperloaded YP of claim 4, wherein the organic solvent is removed from the hyperloaded YP.

9. The hyperloaded YP of claim 1, further comprising a temperature stabilizing agent that is optionally glycerin.

10. (canceled)

11. The hyperloaded YP of claim 1, wherein the aqueous solution further comprises a surfactant that is optionally selected from the group consisting of sodium lauryl sulphate, polysorbate 20, polysorbate 80, polysorbate 40, polysorbate polyglyceryl ester, polyglyceryl monooleate, decaglyceryl monocaprylate, propylene glycol dicaprilate, triglycerol monostearate, polyoxyethylenesorbitan, monooleate, TWEEN®, SPAN® SPAN® 40, SPAN® 60, SPAN® 80, IGEPAL®, Triton X-100, Neobee, lecithin, Pluronic 31R1, Pluronic 17R4 and Brij 30.

12. (canceled)

13. The hyperloaded YP of claim 1, wherein the hydrophobic payload is selected from the group consisting of a terpene, a terpenoid, eugenol, geraniol, thymol, clomazone, triallate, limonene, lambda-cyhalotrin, penthiopyrad (PTP), prothioconazole (PRO), spinosad, tetrahydrocannabinol (THC), cannabinol, cannabidiol, cannabigerol (CBG), fish oil, aminoglycoside antibiotics, gentamycin, kanamycin, macrolides, erythromycin, rifamycins, novobiocin, fusidic acid, cationic peptides, cycloserine, rifampicin, doxorubicin, epirubicin, idarubicin, mitoxantrone, aspirin, acetaminophen, d-propoxyphene, fenoprofen, flurbiprofen, ibuprofen, ketoprofen, naproxen, naproxen-anhydride, oxaprozin, small organic active agent, a small inorganic active agent, a microbicide, a fungicide, an insecticide, a nematicide, a pesticide, an antibiotic, an analgesic, a non-steroidal anti-inflammatory drug (NSAID), a chemotherapeutic, a dietary supplement, and a mixture thereof.

14. The hyperloaded YP of claim 1, wherein the hyperloaded YP has a diameter that is greater than the diameter of a non-hyperloaded YP, optionally wherein the diameter of the hyperloaded YP is between about 6 μm and about 10 μm

15. (canceled)

16. A pharmaceutical composition comprising the hyperloaded YP of claim 1 and a pharmaceutically acceptable carrier or excipient.

17. A method of preparing a hyperloaded yeast particle (YP) comprising the steps of:
a. hydrating a YP with at least 0.5 μL aqueous solution per milligram of YP; and
b. incubating the hydrated YP with a hydrophobic payload to encapsulate the hydrophobic payload within the YP.

18. The method of claim 17, wherein the aqueous solution comprises a stabilizing agent that is optionally glycerin.

19. (canceled)

20. The method of claim 17, further comprising a step of dissolving the hydrophobic payload in a solvent before incubating the hydrated YP.

21. The method of claim 20, wherein the solvent is an organic solvent.

22. The method of claim 21, wherein the organic solvent is selected from the group consisting of acetone, dichloromethane, ethyl acetate, ethanol, methanol, dimethyl sulfoxide (DMSO), eugenol, geraniol, a mixture of geraniol (G), eugenol (E), thymol (T) at a weight ratio composition of about 2:1:2 (GET424), N,N-dimethyl-decanamide (DMDA), octanoic acid, lauric acid, undecanoic acid, glycofurol, vitamin E, fatty acid, medium chain triglycerides (MCT), and a mixture thereof.

23. The method of claim 20, further comprising a step of removing the solvent after the incubating step.

24. The method claim 17, wherein:
the weight by weight (w/w) ratio of the hydrophobic payload:the hyperloaded YP is between about 2:1 and about 5:1, or the hyperloaded YP has a diameter that is greater than the diameter of a non-hyperloaded YP, that is optionally is between about 6μm and about 10 μm.

25. (canceled)

26. (canceled)

27. A method of delivering a hydrophobic payload to a subject in need thereof comprising administering to the subject the hyperloaded YP of claim 1.

28. A composition for agricultural or environmental application comprising the hyperloaded YP of claim 11

29. A kit comprising the hyperloaded YP of claim 1, and optional instructions for use.
